(19) United States
(12) Patent Application Publication
(10) Pub. No.: US 2006/0088574 A1
(43) Pub. Date: Apr. 27, 2006

(54) Nutritional supplements

(76) Inventors: Paul B. Manning, Keswick, VA (US); Raymond J. Maggio, Collegeville, PA (US)

Correspondence Address: NIXON & VANDERHYE, PC, 901 NORTH GLEBE ROAD, 11TH FLOOR, ARLINGTON, VA, 22203, US

(21) Appl. No.: 10/973,210

(22) Filed: Oct. 25, 2004

(51) Int. Cl. A61K 47/00

(52) U.S. Cl. 424/439

(57) ABSTRACT

The invention provides nutritional supplements for an oral or enteral administration to humans, whether in satisfactory health, or having a medical condition. These supplements can provide humans with a complete, balanced nutrition in a bioavailable form, either as a food supplement or as a meal replacement, and preferably includes one or more slow-digesting carbohydrates that renders them effective in maintaining blood glucose levels at, or returning abnormal blood glucose levels to, normal blood glucose levels. Nutritional supplements within the invention can be employed with humans that are diabetic, borderline diabetic, pregnant and/or lactating, geriatric humans and humans that have, are at risk for, or develop other glucose intolerance or cardiovascular disease.

This patent application is a non-provisional patent application that claims the benefit of U.S. Provisional Patent Application No. ______, filed on Oct. 20, 2004, by inventors Paul B. Manning and Raymond J. Maggio, entitled “Nutritional Supplements.” The entirety of such provisional patent application is incorporated into the present non-provisional patent application by reference.

### BACKGROUND OF THE INVENTION

1. Field of the Invention

The present invention relates to nutritional supplements, such as nutritional ready-to-drink beverages, liquid concentrates, solid concentrates and food bars, for an administration to humans, whether they are in satisfactory or normal health, or have one or more diseases or disorders. Nutritional supplements within the invention may be orally consumed by humans and/or administered enterally.

The nutritional supplements of the invention are particularly useful for a dietary management and/or control of humans that are at risk for, have been diagnosed with, or may develop diabetes mellitus (“diabetes”) or cardiovascular disease, that are borderline diabetic, that otherwise experience glucose intolerance or hyperglycemia, or that are a member of the geriatric population. The nutritional supplements are also particularly useful for the management, control and/or normalization of the blood glucose levels of such humans, and in the prevention and/or treatment of borderline diabetes, diabetes, cardiovascular disease and other glucose intolerance.

The nutritional supplements of the invention can provide a complete, balanced nutrition in a bioavailable form, particularly to borderline diabetic, diabetic or geriatric humans, or to humans having cardiovascular disease or other forms of glucose intolerance, as a food supplement, a snack and/or a meal replacement.

The nutritional supplements of the invention preferably include a blend of two or more slow-digesting carbohydrates, which renders them effective for maintaining, or aiding in the maintenance of, blood glucose levels at normal levels, or returning, or aiding in the returning of, abnormal blood glucose levels to normal blood glucose levels, in humans.

By providing useful nutrients, such as one or more omega-3 fatty acids, for example, DHA (docosahexaenoic acid), and the amino acid L-arginine, which have strongly suggestive roles in decreasing a risk of cardiovascular disease and aiding in the mitigation of inflammation, the nutritional supplements of the invention may also aid borderline diabetic, diabetic or otherwise glucose intolerant humans in avoiding elevated blood glucose levels that could otherwise cause them to experience undesirable symptoms or diabetes-related complications.

The nutritional supplements of the invention are effective for aiding in the: (a) management, control and/or normalization of blood glucose levels in humans; (b) management and control of diabetes and other glucose intolerance; and/or (c) prevention, or reduction in severity, of complications related to, or induced by, diabetes or other glucose intolerance. The nutritional supplements may also be consumed by humans that are in normal or satisfactory health, or that are suffering from one or more other illnesses, diseases and/or disorders.

2. Background

Glucose and Insulin

The main energy source for all work performed by the human body is sugar in the form of glucose, which is dissolved in the blood and stored in the liver and muscles. Glucose is a simple monosaccharide sugar, and is one of the most important carbohydrates.

The carbohydrates (sugars) that humans consume are digested and absorbed into the bloodstream. Some of this sugar travels to the liver, where the sugar is stored for use at a subsequent time. The liver releases sugar into the bloodstream, and keeps the bloodstream supplied with sugar until more food is consumed. The liver is supposed to release into the bloodstream a sufficient quantity of sugar to replace the sugar that is being used by the body. The quantity of sugar being used by the body, thus, is generally about the same quantity of sugar that is being released into the bloodstream by the liver. As a result, blood sugar levels generally remain constant.

Insulin is a double-chain protein hormone that is formed from proinsulin in the beta cells of the pancreatic islets of Langerhans. Insulin is secreted into the blood in response to a rise in concentration of glucose in the blood. Insulin promotes the storage of glucose in the liver, skeletal muscle and adipose tissue, and is necessary for the body to be able to use sugar, which is the basic fuel for cells in the body. Insulin is necessary for the body to be able to use glucose that enters into the bloodstream after eating. It transports the sugar from the bloodstream into cells, and acts as a messenger to inform the liver of the quantity of sugar that should be released into the bloodstream.

If the body produces too much insulin, blood sugar levels fall, resulting in typical symptoms of fainting and/or convulsions (hypoglycemia). An elevated blood sugar level, on the other hand, can be attributable to a reduced insulin formation, as occurs in diabetes (hyperglycemia).

Insulin and other diabetes medications are often administered to diabetic or other glucose intolerant humans by injection, or through use of an insulin pump, in order to lower blood glucose levels.

Glucose Intolerance and Diabetes

Several distinct disorders of glucose tolerance presently exist. The most widely used classification of diabetes mellitus and other categories of glucose intolerance is that recommended by the World Health Organization (WHO) in 1985. Recently, the American Diabetes Association (ADA) proposed a system based upon disease etiology instead of classification according to type of pharmacological treatment.

The major categories of the disorders of glucose tolerance (glycemia) are type 1 diabetes mellitus, type 2 diabetes mellitus, other specific types of diabetes, gestational diabetes mellitus (GDM), impaired glucose tolerance (IGT) and impaired fasting glucose (IFG).

Diabetes mellitus (“diabetes”) is a medical disorder that is characterized by varying or persistent hyperglycemia (elevated blood sugar levels), especially after eating. Hypoglycemia (low blood sugar) is rarely a feature of diabetes, except as an accident of treatment (usually a misapplication of medication in particular circumstances). Although different types of diabetes exist, most are asymptomatic for a variable time after onset, and all types generally share similar symptomatology and complications at advanced stages.

Inflammation and excessive weight (above-normal body weight) are each important risk factors that may cause a transformation of a pre-diabetic, or borderline, diabetic condition into diabetes. Inflammation is also believed to cause complications that are associated with diabetes, such as eye and kidney disease.

### Type 1 Diabetes

“Type 1 diabetes” is usually diagnosed in children and young adults, and was previously known as “juvenile diabetes.” In type 1 diabetes, the body does not produce insulin, generally causing the liver to release sugar into the bloodstream too rapidly, and the blood sugar levels to rise.

Conditions associated with type 1 diabetes include hyperglycemia, hypoglycemia, ketoacidosis and celiac disease. Having type 1 diabetes increases a human's risk for many serious complications, such as cardiovascular disease, diabetic retinopathy (blindness), neuropathy (nerve damage) and kidney damage (nephropathy). Although type 1 diabetes is serious, humans that have this type of diabetes can learn how to live long and healthy lives.

### Type 2 Diabetes

“Type 2 diabetes” is the most common form of diabetes. In type 2 diabetes, either the body does not produce enough insulin, or the cells ignore the insulin, both of which generally cause the liver to release sugar into the bloodstream too rapidly, and the blood sugar levels to rise. When glucose accumulates in the blood, rather than entering into cells, the glucose can cause two problems. First, the cells may immediately be starved for energy. Second, over time, the high blood glucose levels may damage a human's eyes, kidneys, nerves and/or heart.

Although diabetes occurs in humans of all ages and races, some groups have a higher risk for developing type 2 diabetes than other groups. Type 2 diabetes is more common in African Americans, Latinos, Native Americans and Asian Americans/Pacific Islanders, as well as in the aged population.

Conditions associated with type 2 diabetes include hyperglycemia and hypoglycemia. Having type 2 diabetes increases a human's risk for many serious complications, such as cardiovascular disease, diabetic retinopathy, neuropathy and nephropathy. While Type 2 diabetes is also serious, humans that have this type of diabetes can also learn how to live long and healthy lives.

Blood Glucose Levels

The concentration of sugar in the blood is reflected in the measurable blood glucose level. In normal circumstances, the blood glucose level is subject to only slight fluctuations as a result of food intake. Maintaining this stable blood glucose level is the task of insulin.

When the blood glucose level is too high, a human may experience undesirable symptoms, for example, frequent urination, increased thirst, fatigue and/or pain in the abdomen. In contrast, when the blood glucose level is too low, for example, as a result of taking insulin or pills to control blood glucose levels, a human may experience other undesirable symptoms, such as shaking, dizziness, sweating, hunger, headaches, mood swings, behavioral changes and/or difficulty paying attention. Further, different humans may experience different symptoms, and blood glucose levels may be affected by other factors, such as illness, exercise, stress and/or excessive heat.

The above (and other) symptoms that may result from blood glucose levels that are too high, or too low, generally are not accurate predictors of a diabetic mammal's blood glucose level. A failure by a diabetic mammal to control blood glucose levels (i.e. to maintain them at a “normal” level at all times) can result in severe ramifications of diabetes, such as damage to large and/or small blood vessels, diabetic retinopathy, diabetic eye disease, neuropathy, cardiovascular disease, nephropathy, kidney disease, renal failure, amputation of a limb (toe, foot or leg) and/or death. Damage to, and narrowing of large blood vessels, can promote cardiovascular disease, stroke and/or gangrene, while damage to small blood vessels can damage the eyes and kidneys.

Gangrene (tissue necrosis) is the death and subsequent decay of body tissues caused by, for example, infection, thrombosis or lack of blood flow. It is most commonly the result of critically insufficient blood supply that may be caused by an injury and subsequent contamination of a wound with bacteria. Gangrene is most common in the extremities. A specific example of gangrene is “diabetic foot,” which can be seen in long-standing complicated diabetes. It is generally caused by a combination of arterial ischemia, injury and poor healing that is rather common in diabetics. The most common medical treatment for irreversible gangrene is amputation (the removal of a body extremity or limb by surgery).

Neuropathic, ischemic and/or neuroischemic foot ulcers, and their complications, are an important cause of morbidity and mortality in diabetes. About 50% of patients that undergo nontraumatic lower-limb amputations have diabetes. These patients generally have a high mortality following amputation, ranging from 39% to 80% at 5 years. Causes of death include cardiac and cerebrovascular disease, pneumonia, septicemia and renal failure. Foot problems in diabetes continue to challenge the clinicians who care for these patients. Not only are they associated with morbidity and disability, but they also lead to significant impairment of the quality of life. A high mortality is well documented in patients with diabetic foot problems following amputation.

Control and Treatment of Diabetes

For about the past 50 years, it has been generally accepted that humans with diabetes have a significantly greater risk of developing heart disease, circulatory disorder, degenerative retinal disease, and other associated serious illnesses that lead to a reduction in quality of life, increased health care costs and, commonly, a reduced life span.

“Short-term” and “long-term” methods for controlling diabetes presently exist. These methods each provide a different set of information, and are both necessary for the proper control of diabetes and, consequently, for lowering a risk of developing the diabetes-related complications described above.

### Blood Glucose Monitoring—“Short Term” Method

The proper control and maintenance of blood glucose levels by a diabetic or borderline diabetic human can have the beneficial effects of helping the human to avoid, or reduce the potential for, having diabetes related complications and significantly extend the life expectancy of the human.

Diabetic humans should rely upon data that is generated by home or other blood glucose monitoring, with blood glucose levels being checked at the same regular times each and every day, in accordance with directions received from a physician or other clinician, and more often if a meal is missed or added, an exercise pattern in changed or the human feels sick or develops one of the symptoms or conditions described herein. It is preferable to check blood glucose levels at least three times per day, preferably prior to each meal and before bed, and additional times when feeling ill, running a fever and/or altering meal habits, activity levels or medication types of dosages. Such monitoring is generally the only reliable method for determining whether or not a diabetic human's blood glucose levels are at “target” (normal) levels, and providing “short-term” control of the human's diabetes.

Maintaining a “tight control” of diabetes means maintaining a blood glucose level as close to “target” (normal) as is possible, and will generally delay the commencement of complications from diabetes, such as eye, kidney, heart and nerve damage or, for a diabetic mammal that has already experienced one or more of these complications, decreases the rate and which these conditions progress. Such results have been shown in a diabetes study conducted from 1983 to 1993 by the National Institute of Diabetes and Digestive and Kidney Diseases, an institute within the National Institutes of Health, known as The Diabetes Control and Complications Trial. The American Diabetes Association states that the goal for “tight” blood glucose control is maintaining blood glucose levels as close to “normal” as is possible, with “normal” levels being 70 to 120 mg/dl before meals, and less than 180 mg/dl two hours after meals. Any blood glucose level over “normal” can lead to long-term diabetes-related complications.

Humans that have a fasting blood glucose level between 110 and 125 mg/dl and/or a glucose level of 140 to 199 mg/dl at the two hour measurement during an oral glucose tolerance test may be considered by a physician to have “borderline diabetes.” Such humans may have abnormalities that make them likely to develop type 2 diabetes.

Only one droplet of blood is generally required to take a blood glucose reading. The droplet of blood is generally taken from a fingertip, applied to a test strip, and quantitatively analyzed using a commercially available blood glucose meter.

### Hemoglobin A1c Blood Test—“Long-Term” Method

Physicians employ the hemoglobin A1c blood test (also known as the GHb and Glycated hemoglobin), which is a simple lab test that uses a small sample of blood, to determine “long-term” control of blood glucose levels. The hemoglobin A1c blood test should be performed about 2 to 3 times per year, and reveals how successful a diabetic patient has been in controlling blood glucose levels over the prior three-month period. The National Diabetics Education Program at the National Institutes of Health recommends maintaining hemoglobin A1c levels under 7%. The risk of complications from diabetes increases substantially when hemoglobin A1c levels rise above 8%. The optimal range for hemoglobin A1c levels is 4-6%.

While treatment for diabetes may vary depending upon the type of diabetes, diabetes is generally treated under the care of a physician with diet, exercise, stopping smoking (for smokers), insulin injections, other diabetes medications or a combination thereof, all of which can affect blood glucose levels. Nutrition and regular exercise (preferably with increasing intensity) are important components of properly managing diabetes. Overweight humans generally should embark on a weight-loss program that helps them lose weight slowly over time, and then keep the weight off. Diabetic humans should generally also start and maintain a regular exercise program that is appropriate for them. The type and dosage of insulin and other diabetes medications may vary depending upon a variety of factors, such as the type and severity of diabetes, the age, weight, sex and status of the human being treated and like considerations, and may be determined by a physician or skilled clinician. It is preferable that a diabetic human maintain a written log of the number of grams of carbohydrates consumed, and the amount of physical activity engaged in, each day. Recording the number of carbohydrate grams eaten during each day reveals whether or not a particular meal plan is being effective.

Successfully keeping blood sugar at “normal” levels at all times, despite the difficulty of doing so (especially at about 0.5 to about 4 hours after eating) has been compellingly shown to reduce, or prevent, the above symptoms and comlications. By properly monitoring, and treating diabetes, a human may avoid or delay other health related problems that are generally related to diabetes in the subsequent years.

Currently, borderline diabetic, diabetic and other glucose intolerant or compromised humans commonly rely upon pharmaceutical and professional medical intervention for either acute or chronic disease treatment. The dietary factor in disease control and management generally is a counseling effort that is a part of healthcare professional care. Unfortunately, however, humans often do not have nutritional food products available to them that fulfill dietary recommendations for their condition, or that contain nutrients, and levels thereof, that are useful in the mitigation of their symptoms and/or conditions.

The disadvantages to a human who cannot consistently have access to effective amounts of such nutrients, such as omega-3 fatty acids (particularly high grade DHA) and L-arginine, is significant in that a dietary management of their condition may be very difficult, or impossible.

Glycemic Index

The glycemic index (GI) is a ranking system for carbohydrates that is based upon their effect on blood glucose levels. It is a measure of carbohydrate absorption, and measures the effects of blood glucose. Carbohydrates that break down quickly during digestion have the highest glycemic indices. The blood glucose response is fast. Carbohydrates that break down slowly, releasing glucose gradually into the blood stream, have low glycemic indices.

Glycemic index values for different foods are calculated by comparing measurements of their effect upon blood glucose compared with a reference food. Scientific publications tend to use glucose as the reference food, so that glucose has a glycemic index value of 100. This has the advantages that it is universal, and results in maximum glycemic index values of about 100, so that glycemic index values can be interpreted intuitively as percentages on an absolute scale.

Glycemic index values are commonly interpreted as follows:
Low glycemic index—glycemic index of less than 55,
Intermediate glycemic index—glycemic index between 55 and 70,
High glycemic index—glycemic index that is higher than 70.

A low glycemic index food will generally release energy slowly and steadily and, thus, is appropriate for diabetics, dieters and endurance athletes. In contrast, a high glycemic index food will generally provide a rapid rise in blood sugar levels and, thus, is suitable for post-endurance exercise energy recovery.

Additional information about diabetes is available from the American Diabetes Association (Alexandria, Va.) at the web site www.diabetes.org, the International Diabetes Federations (Brussels, Belgium) at the web site www.idf.org and from the National Certification Board of Diabetes Educators (Arlington Heights, Ill.) at the web site www.ncbde.org.

Nutritional and Medical Benefits Provided by Edible Fats and Oils

Edible fats and oils generally provide nutritional and health benefits to humans. Fats are one of the three main classes of food, and are the most concentrated form of metabolic energy available to humans. Vitamins A, D and E are fat soluble, and occur only in conjunction with fats. Fats are sources of essential fatty acids, an important dietary requirement, as well as other nutritious fatty acids.

Clinical studies have shown that certain edible fats and oils, such as DHA (an “omega-3” fatty acid) and other fatty acids, can also provide significant medical benefits to humans, particularly to human beings. For example, omega-3 fatty acids are associated with a lower risk of coronary heart disease. [Frank B. Hu, M. D. et al., “Types of Dietary Fat and Risk of Coronary Heart Disease: A Critical Review,” J Am Coll Nutr. 20(1): 5-19 (2001).]

Other publications that discuss the nutritional, health and/or medical benefits provided by edible fats and/or oils include Elda Hauschildt, “Alpha-Linolenic Acid may help Prevent Heart Disease,” Am J Clin Nutr. Vol. 75, 221-227 (2002); Yvonne E. Finnegan et al., “Plant- and Marine-Derived n-3 Polyunsaturated Fatty Acids have Differential Effects on Fasting and Postprandial Blood Lipid Concentrations and on the Susceptibility of LDL to Oxidative Modification in Moderately Hyperlipidernic Subjects,” Am J Clin Nutr., Vol. 77, 783-795 (2003); Rozenn N. Lemaitre et al., “n-3 Polyunsaturated Fatty Acids, Fatal Ischemic Heart Disease, and Nonfatal Myocardial Infarction in Older Adults: the Cardiovascular Health Study,” Am J Clin Nutr., Vol. 77, 319-325 (2003); Dayong Wu et al., “Effect of Dietary Supplementation with Black Currant Seed Oil on the Immune Response of Healthy Elderly Subjects,” Am J Clin Nutr. Vol. 70(4), 536-543 (1999); Frank B. Hu et al., “Dietary Intake of α-Linolenic Acid and Risk of Fatal Ischemic Heart Disease Among Women,” Am J Clin Nutr. Vol. 69, 890-897 (1999); and E H Temme et al., “Comparison of the Effects of Diets Enriched in Lauric, Palmitic, or Oleic Acids on Serum Lipids and Lipoproteins in Healthy Women and Men,” Am J Clin Nutr., Vol 63, 897-903 (1996).

Further, in cooking, products having a high fat content, such as butter, milk, cheese and other dairy products, are often employed as enhancers of taste.

Fatty Acids

Fatty acids, such as “omega-3” fatty acids (also known as “n-3” fatty acids), “omega-6” (n-6) fatty acids, “omega-9” (n-9) fatty acids and/or essential fatty acids are generally present in high levels in various edible fats and/or oils.

“Omega-3 fatty acids” are the n-3 family of polyunsaturated fatty acids, and are called “n-3 fatty acids” because the first double bond occurs in the third carbon bond counting from the end or omega position of the fatty acid. Omega-3 fatty acids have many nutrition, health and/or medical benefits associated with them and include, for example, docosahexaenoic acid (DHA), docosapentaenoic acid, alpha-linolenic acid (ALA), eicosapentaenoic acid (EPA), eicosatetraenoic acid, moroctic acid and heneicosapentenoic acid.

Omega-3 fatty acids are precursors of eicosanoids (prostaglandins, thromboxanes and leukotrienes), which are signal substances (cell messengers) that have a widely different effect upon biological activity. Many of these signal substances regulate physiological and immunological reactions.

Omega-3 fatty acids have been shown to be beneficial in the prevention of cardiovascular pathology, the reversal of atherosclerosis, the inhibition of tumor formation and the development and the regulation of serum cholesterol. Prospective cohort studies and secondary prevention trials have provided strong evidence that an increasing intake of n-fatty acids from fish or plant sources substantially lowers risk of cardiovascular mortality. [Frank B. Hu, M. D. et al., “Types of Dietary Fat and Risk of Coronary Heart Disease: A Critical Review,” supra.] Other research suggests the therapeutic value of oils rich in omega-3 unsaturated fatty acids for disorders related to blood hyperviscosity, including the lowering of serum cholesterol and the suppression or reduction of plasma triglycerides, inflammatory autoimmune disorders, tumors and various other disorders. [D. F. Horrobin, “Clinical uses of Essential Fatty Acids,” Eden. Press, London, 1982.] The omega-3 fatty acids also suppress the production of the proinflammatory cytokines tumor necrosis factor (TNF), particularly TNF alpha, interleukin-1 (IL-1) and thromboxanes.

The protective effects of n-3 fatty acids are likely a result of multiple mechanisms, including reducing triglyceride levels, reducing platelet aggregation and antiarrhythmic effects.

Further, omega-3 fatty acids are essential for the normal development of an unborn baby's brain, especially during the third trimester, when the size of a baby's brain increases threefold. If a baby's mother fails to have a sufficient quantity of these nutrients in her diet, the fetus will generally depend upon the mother's brain tissue and tissue storage of these nutrients. Lab tests have shown that new mothers have approximately one half of the normal blood levels of omega-3 fatty acids.

Linolenic acid, a polyunsaturated fatty acid having three double bonds, is a precursor to DHA, and is considered to be a dietary essential fatty acid. Because the body is not capable of synthesizing linolenic acid, it must be acquired from a food source.

DHA is a long-chain omega-3 fatty acid that is associated with brain development and growth, and that is necessary for brain and eye development, growth and learning ability in children. DHA is a major structural component of the brain, and accumulates during the fetal period and during the first year after birth. [A. P. Simopoulos, “Omega-3 Fatty Acids in Health and Disease and in Growth and Development,” American Journal of Clinical Nutrition 54, No. 3, 438-463 (1991).] Breast milk is a vital source of this nutrient during early development.

DHA is the primary structural fatty acid in the gray matter of the brain, and in the retina of the eye. It is essential for normal visual and neurological (nervous system) development in infants, and for normal brain and eye function in adults. Significant brain and eye development occurs in utero, and continues during the first year after birth. The DHA content of the fetal brain increases three to five times during the final trimester of pregnancy and triples yet again during the first week of life. Thus, an adequate dietary intake of DHA is particularly important for pregnant and nursing women. Infants rely on their mothers to supply DHA for their developing brain and eyes initially through the placenta, and subsequently through the breast milk.

The human body only synthesizes small quantities of DHA. As a result, it is necessary to obtain DHA from dietary sources.

DHA can alter eicosanoid and cytokine production, providing an improved immunocompetence (strengthening immune system activity) and a reduced inflammatory response to injury. The contribution of DHA to reducing the incidence of numerous inflammatory/circulatory disorders, cardiac problems, premature births, cognitive ability in children and mental well being has been well documented.

ALA is an essential omega-3 fatty acid for humans. Adequate intake of ALA and long-chain omega-3 fatty acids is especially important for infants, young children and patients requiring enteral and enteral nutrition. Experimental studies have suggested an antiarrhythmic effect of ALA, and beneficial effects of ALA on cardiovascular disease. [Frank B. Hu, M. D. et al., “Types of Dietary Fat and Risk of Coronary Heart Disease: A Critical Review,” supra.]

“Omega-6” fatty acids include gamma-linolenic acid, which is present in Black Current Seed Oil, linoleic acid, which is present in many vegetable oils, and arachidonic acid, which is present in many animal fats and in algae oil.

“Omega-9” fatty acids include, for example, oleic acid, which is present in sunflower oil, olive oil, avocados, canola oil and in many animal fats.

As a result of the numerous nutritional, health and/or medical benefits that they have an ability to provide to humans, whether the humans are healthy, pregnant, lactating, breast-fed, diabetic, borderline diabetic, otherwise glucose intolerant and/or have one or more other diseases, illnesses and/or disorders, it would be beneficial to supplement nutritional supplements, such as ready-to-drink beverages, with one or more omega-3 and/or other fatty acids, with edible fats and/or oils containing these fatty acids and/or with other nutritious and/or medically beneficial edible fats and/or oils.

Marine Oils and Hazardous Chemicals

### Marine Oils

Marine oils (including “fish oils”) are oils that are obtained from aquatic animals, plants or organisms, either directly or indirectly, particularly from oily fish. Marine oils include, for example, herring oil, cod oil, anchovy oil, tuna oil, sardine oil, menhaden oil and algae oil. Animals that are employed to produce marine oils include, for example, farm-raised or wild, fresh-water or salt-water, fish and shellfish, such as herring, salmon, salmonoids, gadoids, shrimp, cod, carp, tilapia, perch, trout, sturgeon, krill, tuna, flat fish, anchovies, sardines, menhaden, shrimp, Mackerel, eels and seals. Marine oils may also be obtained from marine organisms, such as calanus (Calanus finmarchicis), a 3-4 mm copepod, algae and microalgae, for example, diatoms and dinoflagellates.

Although omega-3 fatty acids can be obtained from other sources, such as plant oils, fish have a unique ability to provide high levels of the omega-3 fatty acids DHA and EPA. Fish and fish oil are also sources of the omega-3 fatty acids docosapentaenoic acid, eicosatetraenoic acid, moroctic acid and heneicosapentenoic acid.

Oils containing omega-3 fatty acids, such as marine oils, are also referred to as “omega-3” oils or “n-3” oils.

In vitro studies have consistently shown that n-3 fatty acids decrease expression of adhesion molecules on the endothelium, and also decrease leukocyte/endothelium interations. Further, clinical experimental studies have shown that n-3 fatty acid supplementation improves endothelial-dependent vasomotor function. [Frank B. Hu, M. D. et al., “Types of Dietary Fat and Risk of Coronary Heart Disease: A Critical Review,” supra.]

### Hazardous Chemicals

Disadvantageoulsy, fish that have fins, and oils that are obtained, or derived, from such fish, such as fish oil, or components of fish oil, may contain mercury and/or other toxic chemicals, which can present extremely serious health hazards for pregnant women, lactating women and/or their fetuses or breast-fed babies.

Additionally, shell fish and other filter feeders, whether freely caught or farm raised, ‘depurate,’ that is, they cleanse themselves if they are contaminated and then placed in clean water. Shellfish have a high propensity to be contaminated with pathogenic material, often concentrate harmful contaminants and microorganisms (Red Tide is especially deadly), and generally maintain that pathogenicity for longer periods of time than that required for depuration (‘red tide’).

Nonvertebrate and carpacious aquatic animals typically do not concentrate contaminates in the same manner or degree due to their different physiology. However, they may also contain impurities.

Acquatic mammals and amphibians have their own set of ‘contaminant’ potentials, which can include their fats and ‘normal’ chemical composition. Acquatic waterfowl that do not eat fish may have a reduced risk of mercury contamination, but an elevated organo comtaminate risk.

Coal-burning power plants produce millions of tons of toxic emissions into the Earth's atmosphere each year. While coal is relatively inexpensive to burn, it is one of the most impure of fuels, and is environmentally very dirty. Burned coal produces millions of pounds of “coal ash” and “fly ash,” solid combustion waste materials that contain highly poisonous and/or radioactive chemicals, such as arsenic, uranium, mercury, lead and thorium. These chemicals can leak out from ash settlement ponds into lakes, rivers, streams, oceans and other bodies of water, contaminating fish and other aquatic animals, and rendering severely ill humans that drink this water, or ingest the contaminated fish (or fish oil obtained or derived from the contaminated fish), and their fetuses and breast-fed babies.

Mercury, which is one of the most toxic substances presently in existence, is present in various metal and other ore strata, and in coal, and becomes released into the atmosphere when these materials are incinerated for example, by a conventional coal-burning combustion process. When the mercury falls to the ground, some of it eventually washes into bodies of water, where water bacteria convert it to methylmercury, a compound that accumulates in the tissues of fish, rendering the fish (and oils obtained or derived from the fish) unsafe for consumption, and that causes nerve damage in humans. Mercury that is present in contaminated fish (and fish oil) destroys nerve cells, and easily crosses the placenta to enter into fetuses. Some mercury compounds can cause brain damage in developing fetuses, as well as in breast-fed babies. Because of the high levels of mercury that may be present in shark and swordfish, in particular, the Federal Food and Drug Administration (“FDA”) has advised pregnant women not to eat these (and other) types of fish.

It is estimated that nearly one half of all mercury in the environment comes from conventional coal-burning power plants. Four hundred conventional coal-burning power plants in 43 states are emitting an estimated 98,000 pounds of mercury into the air each year.

An article entitled “How Safe is your Food? One Fish, Two Fish, Red Snapper, Swordfish: A Menace Lurks in your ‘Healthy’ Meal,” appearing in the August of 2003 Readers' Digest magazine describes experiences that a respected internist, Dr. Jane Hightower, had with more than 100 of her ill patients who had been regularly eating fish or fish products (swordfish, halibut, tuna, trout, bass, salmon, mackerel and/or cod liver capsules). These patients were experiencing symptoms including hair loss, loss of memory (dubbed “fish fog”), an inability to focus, twitching, uncontrollable trembling, nausea, dizziness, joint and muscle aches, stomach cramps, hangover-like headaches and/or insomnia. Dr. Hightower, who had heard a radio report linking hair loss with mercury, had more than 123 of her patients tested for mercury exposure. She determined that 9 out of 10 patients were determined to have blood mercury levels higher than the level that the U.S. Environmental Protection Agency (“EPA”) deems to be safe (a limit of 5 micrograms of mercury per liter of blood). Over half of the patients had mercury concentrations more than double that amount, with many having mercury concentrations that were 3 or 4 times higher. Some of the patients had blood mercury levels that were from 6 to 15 times higher than the level deemed to be safe by the EPA. When Dr. Hightower advised her patients to stop eating fish and fish products, and urged breast-feeding mothers to “pump and dump” their breast milk until their mercury levels bottomed out, the mercury levels of her patients plunged, and their symptoms generally disappeared.

The article states (page 67):

“Among the biggest polluters are coal-burning plants and waste incinerators. The mercury rains into oceans, lakes and streams, where bacteria convert it to methylmercury, an easily absorbable toxin. It collects in the tissues of waterfowl, marine mammals and fish. And the biggest, oldest fish—particularly those that eat other fish—top the aquatic mercury charts.

Scientists have known for years that eating contaminated fish is the way most people absorb methylmercury. At high levels, the neurotoxin can cause birth defects, brain and kidney damage, vision loss, difficulty walking, slurred speech, a metallic taste in the mouth and tingling in the hands and feet. Some of the worst examples of mercury poisoning came from an infamous 1950s case. A chemical factory in the town of Minamata, Japan, secretly dumped tons of mercury into the bay, contaminating the fish that villagers ate every day. Babies were born blind, deaf and horribly twisted; thousands of people were sickened and scores died.

Recent studies suggest mercury may raise the risk of heart attack. Also, some experts are beginning to draw links between mercury and autism in children and Alzheimer's disease in adults. But mercury's clearest threat is to fetuses and infants. It seeps into every cell in the body, and has a nasty affinity for the just-forming fetal brain, where it impedes cell division, permanently stunting the mind.

. . . And, in a cruel twist of nature, mercury is excreted through breast milk. (Human waste releases mercury as well.) Earlier this year, the U.S. Centers for Disease Control (CDC) announced that one in 12 women of childbearing age have mercury levels high enough to put an estimated 300,000 babies at risk for learning disorders and other developmental problems.”

The article further states that, citing this research, the state of California now requires mercury warnings at fish counters, and is calling for restaurants that serve fish to post warnings. Further, 45 states have issued fish advisories about mercury, and 10 states urge women and children to avoid eating canned tuna.

Fungal Oils

Various modified or unmodified fungi, such as filamentous fungi, have an ability to produce lipids having high levels of nutrients, such as γ-linolenic acid (GLA), an n 18:3 omega-6 polyunsaturated fatty acid, and other fatty acids. Fungi can be isolated from soil and subsequently fermented using known techniques and conventional shake flasks or fermentation systems.

GLA has been used in the amelioration of various diseases, such as eczema, rheumatoid arthritis and premenstrual syndrome. It has also been shown to improve the effectiveness of cancer chemotherapy.

Plant Oils

Many plant oils (including vegetable oils, plant seed oils, algae oils, microalgae oils and oils from other seaweeds), such as Evening Primrose oil, Black Currant seed oil, Borage oil, Borage seed oil, safflower oil, sunflower oil, peanut oil, olive oil, corn oil, soybean oil, coconut oil, palm oil, palm kernel oil, rapeseed oil, flaxseed (linseed) oil, cotton seed oil, and algae oil, contain high levels of nutrients, such as GLA or DHA, as well as other fatty acids. For example, flaxseed oil, rapeseed oil and soybean oil contain a large quantity of ALA (about 20% in flaxseed oil and about 7% in unhydrogenated soybean oil). GLA is present in Evening Primrose oil, Black Currant seed oil and Borage seed oil, with the highest level of GLA being present in Borage seed oil. Safflower oil and sunflower oil are rich in linoleic acid. Olive oil contains a significant amount of oleic acid.

Numerous metabolic studies have shown strong cholesterol-lowering effects for vegetable oils that are rich in linoleic oil when substituted for dietary saturated fat. In addition, animal studies have suggested an anti-arrhythmic effect when sunflower oil (rich in linoleic acid) was consumed. [Frank B. Hu, M. D. et al., “Types of Dietary Fat and Risk of Coronary Heart Disease: A Critical Review,” supra.]

Plant oils can be extracted from plants or seeds using techniques that are known by those of skill in the art. For example, the highest quality Borage seed oil is generally extracted without hexane or the use of other chemical solvents. Rather, the Borage seed oil is “cold processed” using an expeller-press method of extraction, which simply squeezes the oil out from the seed without the use of heat.

### DESCRIPTION OF RELATED ART

Glucerna is a nutritional beverage that is sold commercially by a division of Abbott Laboratories (Abbott Park, Ill.). In contrast with the nutritional supplements of the invention, and in addition to other differences between the two formulations, Glucerna does not contain DHA, L-arginine or L-carnitine, and contains only 50% of the U.S. RDI for folic acid.

U.S. Pat. No. 4,214,996 discloses mineral containing compositions that are stated to have a high aqueous dispersibility and to permit nutritionally important elements such as calcium, magnesium, phosphorous, potassium, iron and certain trace elements to be added in aqueous form to foods of all types without adversely affecting taste qualities.

U.S. Pat. No. 4,571,391 discloses a composition comprising a mixture of: (a) chromium (III) tris-acetylacetonate; and (b) insulin, in an amount sufficient to maintain blood glucose at desired levels.

U.S. Pat. No. 4,915,962 discloses a culinary seasoning composition that is formulated such that a portion of from about 5 to about 15 grams of the composition contains at least a recommended daily dietary allowance of at least one of the trace elements zinc, copper, manganese, chromium, selenium or molybdenum, or a mixture of two or more such compounds.

U.S. Pat. No. 4,921,877 discloses a nutritional formula for use in the dietary management of patients with glucose intolerance containing a fiber-containing carbohydrate blend at a relatively low concentration, a fat blend at a relatively high concentration, protein, carnitine, myoinositol, vitamins and minerals, including chromium.

U.S. Pat. No. 5,108,767 discloses a liquid nutritional product for persons that are receiving renal dialysis.

U.S. Pat. No. 5,221,545 discloses a method of providing selenium in the form of selenate in a nutritional product, and nutritional products that contain selenate.

U.S. Pat. No. 5,340,834 discloses a method for increasing muscle mass, and decreasing fat, in chickens that includes administering orally to a chicken a biologically effective amount of chromium citrate.

U.S. Pat. No. 5,869,118 discloses liquid nutritional products comprising gellan gum at a concentration of between 10-5,000 parts per million, and that are stated to have improved physical stability.

U.S. Pat. No. 5,908,647 and U.S. Pat. No. 6,066,344 disclose a nutritional powdered premix for use in preparing nutritional products comprising: (a) a carrier powder containing at least one non-chromium trace or ultratrace mineral; and (b) a carboxylic acid complexed chromium (III) powder substantially uniformly dispersed within the carrier powder, the complexed chromium (III) powder being sufficiently complexed to substantially resist formation of insoluble chromium-containing compounds, and wherein the nutritional powdered mixture comprises sufficient water content to substantially resist formation of insoluble chromium-containing compounds.

U.S. Pat. No. 6,569,445 B2 discloses food bars for consumption by pregnant women, lactating women or women of child-bearing potential that are attempting to become pregnant containing one or more vitamins and/or minerals and one or more anti-constipation and regularity-maintaining agents, and methods for preparing such food bars.

U.S. Pat. No. 6,576,253 B2 discloses food bars for consumption by pregnant women, lactating women or women of child-bearing potential that are attempting to become pregnant containing one or more vitamins and/or minerals, DHA, one or more DHA taste-masking agents and, optionally, one or more anti-constipation and regularity-maintaining agents, and methods for preparing such food bars.

Although some infant formulas, such as Bright Beginnings, Enfamil, Lipil and Similac contain high quality DHA obtained from vegetable sources, infant formulas are not useful in the maintenance or control of the pre-diabetic, diabetic, glucose intolerance or cardiovascular conditions descried herein.

Currently, no nutritional supplements are known that have the unique formulations, characteristics and cardioprotective attributes that are described herein.

A need currently exists for nutritional supplements that may be safely orally consumed by, or enterally administered to, diabetic, borderline diabetic, geriatric or other glucose intolerant humans, and humans that have, or are at risk for, cardiovascular disease, whether or not pregnant, attempting to become pregnant or lactating, and that can provide a complete, balanced nutrition to such humans, that can aid such humans in maintaining their blood glucose levels at, or returning abnormal blood glucose levels to, normal levels, that can help prevent, or reduce the liklihood of, diabetic, borderline diabetic or otherwise glucose intolerant humans from experiencing one or more symptoms and/or complications relating to, or produced by, diabetes, that are easily digestable, that have a pleasant taste and smell, and that have a long shelf life under room temperature conditions.

Further, in view of the significant nutritional, medical and/or other health benefits provided by edible fats and oils, such as the omega-3 fatty acids, including DHA and ALA, it is beneficial to incorporate edible fats and oils into the diet of humans, and to supplement food products with edible fats and oils.

### SUMMARY OF THE INVENTION

The present invention provides pleasant tasting and smelling nutritional supplements for an oral consumption by, or enteral administration to, humans, particularly humans that are diabetic, borderline diabetic or in the geriatric population, or that otherwise have, or are at risk for, glucose intolerance or cardiovascular disease, whether or not pregnant, attempting to become pregnant or lactating.

The nutritional supplements of the invention are designed to assist in the dietary prevention, management and control of borderline diabetes, diabetes, other glucose intolerance, cardiovascular disease, and other adverse health conditions, and advantageously can provide a complete, balanced nutrition to humans that have one or more of these conditions, as well as to healthy humans. These nutritional supplements provide a dietary tool for humans that aids in maintaining blood glucose levels at, or returning abnormal blood glucose levels to, normal levels. Additionally, these nutritional supplements can aid in preventing, or reducing the likelihood, borderline diabetic, diabetic or otherwise glucose intolerant humans from having, or developing, one or more symptoms and/or complications relating to, or produced by, diabetes or other glucose intolerance.

In one aspect, the present invention provides a ready-to-drink liquid nutritional supplement for an administration to a human comprising:
(a) one or more omega-3 fatty acids, in a combined amount that is safe for consumption by humans, and that is effective for providing, maintaining or enhancing a health of a human's eyes, cardiovascular system, renal system, immunological system or neurological system, or for providing one or more other nutritional, health or medical benefits to the human, wherein the one or more omega-3 fatty acids are obtained from, or present in, a source that does not contain a level of mercury that is detectable and harmful, a level of any other hazardous heavy metal that is detectable and harmful, a level of a pesticide that is detectable and harmful or a level of an industrial chemical that is detectable and harmful, and wherein the source contains at least about 10 weight percent of the one or more omega-3 fatty acids;
(b) folic acid, in an amount that is safe for consumption by humans, and is safe for developing fetuses or breast-fed babies of pregnant or lactating women, and that is effective for maintaining, or enhancing, cardiovascular function and/or neurological function in humans, preventing, or reducing the severity of, neural tube defects of fetuses that are developing in pregnant women, enhancing a break down of homocysteine in the body and/or providing a benefit related to insulin sensitivity;
(c) lutein, in an amount that is safe for consumption by humans, and is safe for developing fetuses or breast-fed babies of pregnant or lactating women, and that is effective for maintaining, or enhancing, eye, skin and/or cardiovascular health;
(d) L-arginine, in an amount that is safe for consumption by humans, and is safe for developing fetuses or breast-fed babies of pregnant or lactating women, and that is effective for enhancing vascular, neurological or immune function in humans or insulin sensitivity in diabetic humans;
(e) L-carnitine, in an amount that is safe for consumption by humans, and is safe for developing fetuses or breast-fed babies of pregnant or lactating women, and that is effective for enhancing a transport of one or more fatty acids into mitochondria present in cells of the human, a metabolism of one or more edible fats and/or oils that are present in the nutritional supplements and/or cardio-pulmonary function in the human;
(f) optionally, in addition to folic acid, one or more vitamins and/or minerals, in an amount that is safe for consumption by humans, and is safe for developing fetuses or breast-fed babies of pregnant or lactating women, and that is effective for providing a nutritional, medical and/or other health benefit to the human and/or for enhancing the general nutrition of the human;
(g) in addition to L-arginine and L-carnitine, one or more proteins and/or amino acids in a combined amount that is safe for consumption by humans, and is safe for developing fetuses or breast-fed babies of pregnant or lactating women, and is effective for providing the human with from about 5% to about 50% of daily energy requirements;
(h) one or more carbohydrates in a combined amount that is safe for consumption by humans, and is safe for developing fetuses or breast-fed babies of pregnant or lactating women, and that is effective for providing the human with from about 25% to about 80% of daily energy requirements;
(i) in addition to the one or more omega-3 fatty acids, one or more fats and/or oils in a combined amount that is safe for consumption by humans, and is safe for developing fetuses or breast-fed babies of pregnant or lactating women, and that is effective for providing a human with from about 10% to about 50% of the human's daily energy requirements, wherein the nutritional supplements contain an amount of trans fat that provides about 10% or less of daily energy requirements, an amount of saturated fat that provides about 10% or less of daily energy requirements, an amount of monounsaturated fats that provides from about 1% to about 20% of daily energy requirements, and an amount of polyunsaturated fats that provides from about 1% to about 10% of daily energy requirements;
(j) in addition to lutein, one or more antioxidant agents in a combined amount that is safe for consumption by humans, and that is safe for developing fetuses or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, and/or enhancing a stability and/or shelf life of the nutritional supplements, or of one or more ingredients included therein;
(k) one or more mouthfeel agents in a combined amount that is safe for consumption by humans, and that is safe for developing fetuses or breast-fed babies of pregnant or lactating women, and that is effective for providing, or enhancing, a stabilization of the nutritional supplements during thermal or other processes, providing a desirable mouthfeel, texture or thickness to the nutritional supplements, and/or enhancing a mouthfeel, texture or thickness of the nutritional supplements;
(l) one or more emulsifiers in a combined amount that is safe for consumption by humans, and is safe for developing fetuses or breast-fed babies of pregnant or lactating women, and that is effective for forming, or aiding in the formation of, an emulsion, suspending solids within the nutritional supplements and/or emulsifying lipids within the nutritional supplements; and
(m) water, in an amount that is sufficient to raise the total weight of the nutritional supplement to 100 percent;
   wherein the nutritional supplement is a ready-to-drink liquid that can be consumed by humans without modification,
   wherein the nutritional supplement is effective for maintaining, or aiding in the maintenance of, a normal blood glucose level, or returning, or aiding in the returning of, an abnormal blood glucose level to a normal blood glucose level, in humans that are borderline diabetic, diabetic or otherwise glucose intolerant; and
   wherein the nutritional supplement is safe for consumption by humans, and is safe for breast-fed babies of lactating women.

In another aspect, the present invention provides a ready-to-drink liquid nutritional supplement for an administration to a human comprising:
(a) DHA, in an amount that is safe for consumption by humans, and is safe for developing fetuses or breast-fed babies of pregnant or lactating women, and that is effective for providing, maintaining or enhancing a health of a human's eyes, cardiovascular system, renal system, immunological system or neurological system, wherein the DHA is obtained from a source that is not an aquatic animal, and wherein the DHA has a high quality;
(b) folic acid, in an amount that is safe for consumption by humans, and is safe for developing fetuses or breast-fed babies of pregnant or lactating women, and that is effective for maintaining, or enhancing, cardiovascular function and/or neurological function in humans, preventing, or reducing the severity of, neural tube defects of fetuses that are developing in pregnant women, enhancing a break down of homocysteine in the body and/or providing a benefit related to insulin sensitivity;
(c) lutein, in an amount that is safe for consumption by humans, and is safe for developing fetuses or breast-fed babies of pregnant or lactating women, and that is effective for maintaining, or enhancing, eye, skin and/or cardiovascular health;
(d) L-arginine, in an amount that is safe for consumption by humans, and is safe for developing fetuses or breast-fed babies of pregnant or lactating women, and that is effective for enhancing vascular, neurological or immune function in humans or insulin sensitivity in diabetic humans;
(e) L-carnitine, in an amount that is safe for consumption by humans, and is safe for developing fetuses or breast-fed babies of pregnant or lactating women, and that is effective for enhancing a transport of one or more fatty acids into mitochondria present in cells of the human, a metabolism of one or more edible fats and/or oils that are present in the nutritional supplements and/or cardio-pulmonary function in the human;
(f) optionally, in addition to folic acid, one or more vitamins and/or minerals, in an amount that is safe for consumption by humans, and is safe for developing fetuses or breast-fed babies of pregnant or lactating women, and that is effective for providing a nutritional, medical and/or other health benefit to the human and/or for enhancing the general nutrition of the human;
(g) in addition to L-arginine and L-carnitine, one or more proteins and/or amino acids in a combined amount that is safe for consumption by humans, and is safe for developing fetuses or breast-fed babies of pregnant or lactating women, and is effective for providing the human with from about 5% to about 50% of daily energy requirements;
(h) one or more carbohydrates in a combined amount that is safe for consumption by humans, and is safe for developing fetuses or breast-fed babies of pregnant or lactating women, and that is effective for providing the human with from about 25% to about 80% of daily energy requirements;
(i) in addition to DHA, one or more fats and/or oils in a combined amount that is safe for consumption by humans, and is safe for developing fetuses or breast-fed babies of pregnant or lactating women, and that is effective for providing a human with from about 10% to about 50% of the human's daily energy requirements, wherein the nutritional supplements contain an amount of trans fat that provides about 10% or less of daily energy requirements, an amount of saturated fat that provides about 10% or less of daily energy requirements, an amount of monounsaturated fats that provides from about 1% to about 20% of daily energy requirements, and an amount of polyunsaturated fats that provides from about 1% to about 10% of daily energy requirements;
(j) in addition to lutein, one or more antioxidant agents in a combined amount that is safe for consumption by humans, and that is safe for developing fetuses or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, and/or enhancing a stability and/or shelf life of the nutritional supplements, or of one or more ingredients included therein;
(k) one or more mouthfeel agents in a combined amount that is safe for consumption by humans, and that is safe for developing fetuses or breast-fed babies of pregnant or lactating women, and that is effective for providing, or enhancing, a stabilization of the nutritional supplements during thermal or other processes, providing a desirable mouthfeel, texture or thickness to the nutritional supplements, and/or enhancing a mouthfeel, texture or thickness of the nutritional supplements;
(l) one or more emulsifiers in a combined amount that is safe for consumption by humans, and is safe for developing fetuses or breast-fed babies of pregnant or lactating women, and that is effective for forming, or aiding in the formation of, an emulsion, suspending solids within the nutritional supplements and/or emulsifying lipids within the nutritional supplements; and
(m) water, in an amount that is sufficient to raise the total weight of the nutritional supplement to 100 percent;
   wherein the nutritional supplement is a ready-to-drink liquid that can be consumed by humans without modification,
   wherein the nutritional supplement does not contain a detectable amount of any ingredient that is obtained from an aquatic animal;
   wherein the nutritional supplement does not contain a detectable amount of eicosapentaenoic acid;
   wherein the nutritional supplement is effective for maintaining, or aiding in the maintenance of, a normal blood glucose level, or returning, or aiding in the returning of, an abnormal blood glucose level to a normal blood glucose level, in humans that are borderline diabetic, diabetic or otherwise glucose intolerant; and
   wherein the nutritional supplement is safe for consumption by humans, including pregnant women, lactating women and women having childbearing potential that are attempting to become pregnant, and are safe for their developing fetuses and breast-fed babies.

In another aspect, the present invention provides a liquid concentrate nutritional supplement for an administration to humans comprising a ready-to-drink liquid nutritional supplement of the invention, wherein an amount of water has been removed from the ready-to-drink liquid nutritional supplement in a manner that is effective for forming a liquid concentrate nutritional supplement from the ready-to-drink liquid nutritional supplement, and wherein the liquid concentrate can be transformed into a ready-to-drink liquid nutritional supplement by an addition of an amount of water that is effective for transforming the liquid concentrate into a ready-to-drink liquid nutritional supplement.

In yet another aspect, the present invention provides a solid concentrate nutritional supplement for an administration to humans comprising a ready-to-drink liquid nutritional supplement of the invention, wherein an amount of water has been removed from the ready-to-drink liquid nutritional supplement in a manner that is effective for forming a solid concentrate nutritional supplement from the ready-to-drink liquid nutritional supplement, and wherein the solid concentrate can be transformed into a ready-to-drink liquid nutritional supplement by an addition of an amount of water that is effective for transforming the solid concentrate into a ready-to-drink liquid nutritional supplement.

In another aspect, the present invention provides a nutritional supplement in the form of a food bar for an administration to a human comprising the same ingredients, and amounts thereof, set forth herein in connection with a ready-to-drink liquid nutritional supplement, with a few exceptions.

First, in contrast with the ready-to-drink liquid nutritional supplement, the moisture content of the food bar preferably is about 10 weight percent of the total weight of the food bar or less, and that more preferably ranges from about 4 to about 5 weight percent (with a chewier food bar generally having a higher moisture content than a hard food bar). Most of the water that is present in the ready-to-drink liquid nutritional supplement (and any other ingredients that are included in the ready-to-drink liquid nutritional supplement, but that are optional, or not included, in the food bar) can be replaced in the food bar with conventional food bar fillers, for example, crisp rice, oatmeal, granola, cereal, dried (or other) fruits, nuts, grains, nougats, candy pieces, chocolate, caramel, marshmallow and/or the like, or a wide variety of combinations thereof.

Second, the amount of the one or more antioxidant agents that are included within the food bar can be larger in comparison with the ready-to-drink liquid nutritional supplement, and generally can be as high as about 5 weight percent of the total weight of the food bar.

Third, in contrast with the ready-to-drink liquid nutritional supplement, one or more gums or other mouthfeel agents may, optionally, be included within the food bar (in the same amounts), but are not required. However, it is preferred that one or more mouthfeel agents in a combined amount of about 0.1 weight percent of the total weight of the food bar be included therein.

In another aspect, the present invention provides a method for providing one or more nutritional, medical or other health benefits to a human comprising administering to the human a nutritional supplement of the invention in an amount that is effective for providing one or more nutritional, medical and/or other health benefits to the human, and for a period of time that is necessary or desirable for providing one or more nutritional, medical and/or other health benefits to the human.

In still another aspect, the present invention provides a method for maintaining, or aiding in the maintenance of, a normal blood glucose level of a diabetic, borderline diabetic or other human that has glucose intolerance comprising administering to the human a nutritional supplement of the invention in an amount that is effective for maintaining, or aiding in the maintenance of, the blood glucose level of the human at a normal level, and for a period of time that is necessary or desirable for maintaining, or aiding in the maintenance of, the blood glucose level of the human at a normal level.

In another aspect, the present invention provides a method for returning, or aiding in the return of, an abnormal blood glucose level of a diabetic, borderline diabetic or other human that has glucose intolerance to a normal blood glucose level comprising administering to the human a nutritional supplement of the invention in an amount that is effective for returning, or aiding in the return of, the blood glucose level of the human to a normal level, and for a period of time that is necessary or desirable for returning, or aiding in the return of, the blood glucose level of the human to a normal level.

In still another aspect, the present invention provides a method for preventing, or reducing the likelihood of, a pre-diabetic or borderline diabetic condition of a human from transforming into diabetes or another glucose intolerance disorder comprising administering to the human a nutritional supplement of the invention in an amount that is effective for preventing, or reducing the likelihood of, the human from having a pre-diabetic or borderline diabetic condition transforming into diabetes or another glucose intolerance disorder, and for a period of time that is necessary or desirable for preventing, or reducing the likelihood of, the pre-diabetic or borderline diabetic condition transforming into diabetes or another glucose intolerance disorder.

In another aspect, the present invention provides a method for preventing, or reducing the likelihood of, a diabetic, borderline diabetic or human that has glucose intolerance from experiencing one or more symptoms or complications related to, or produced by, diabetes or glucose intolerance comprising administering to the human a nutritional supplement of the invention in an amount that is effective for preventing, or reducing the likelihood of, the human from experiencing such symptoms or complications, and for a period of time that is necessary or desirable for preventing, or reducing the likelihood of, the human from experiencing such symptoms or complications.

In still another aspect, the present invention provides a method for preventing, or reducing the likelihood of, a human from having pre-mature cardiovascular disease comprising administering to the human a nutritional supplement of the invention in an amount that is effective for preventing, or reducing the likelihood of, a human from having pre-mature cardiovascular disease, and for a period of time that is necessary or desirable for preventing, or reducing the likelihood of, a human from having pre-mature cardiovascular disease.

### BRIEF DESCRIPTION OF THE DRAWINGS

FIG. 1 is a schematic drawing showing one of the processes that may be employed in preparing a nutritional supplement of the invention in the form of a food bar.

FIG. 2 is a schematic drawing showing one of the processes that may be employed in preparing a nutritional supplement of the invention in the form of a ready-to-drink liquid.

FIG. 3 is a schematic drawing showing one of the processes that may be employed in preparing a nutritional supplement of the invention in the form of a solid concentrate.

### DETAILED DESCRIPTION OF THE PREFERRED EMBODIMENTS

The present invention may be understood more readily by reference to the following detailed description of the preferred embodiments of the invention, and to the examples included therein.

Definitions

For purposes of clarity, various terms and phrases used throughout this specification and the appended claims are defined in the manner set forth below. If a term or phrase used in this specification, or in the appended claims, is not defined below, or otherwise in this specification, the term or phrase should be given its ordinary meaning.

The phrase “anti-constipation and regularity-maintaining agent” as used herein means any naturally-occurring substance, chemical, medicine, food product component, food, or other substance, whether natural, by prescription or over-the-counter, that is safe for consumption by humans, including diabetic, borderline diabetic, geriatric, pregnant and lactating humans, humans that have cardiovascular disease and/or women having childbearing potential that are attempting to become pregnant, and that is safe for the developing fetuses and breast-fed babies of pregnant or lactating women, and aids in maintaining regular bowel movements and/or reduces or eliminates constipation (infrequent or difficult bowel movements). (Generally, bowel movements are considered to be “regular” when they occur once or more every three days, and preferably once or more every two days, and more preferably once or more every day. However, the regularity of bowel movements differs for different individuals. Bowel movements that may be considered to be regular by one individual may not be considered regular by another individual.)

The phrase “aquatic animals” as used herein means fin fish (fish having one or more fins), shellfish, crustaceans, filter feeders and similar types of animals that live in water. Aquatic animals do not include, for example, aquatic or non-aquatic plants, algae, microalgae, other types of seaweed, fungi and the like.

The terms “baby” and “babies” as used herein, where appropriate, for example, in connection with their development, or with lactating or breastfeeding women, includes babies, infants, toddlers and/or other children, whether breast-fed or not breast-fed. For instance, as used herein, the term “breast-fed babies” includes babies, infants, toddlers and/or other children that are breast-fed.

The phrase “beverage” as used herein means a substance that is not in solid or gas form, such as a liquid or semi-liquid, and that is designed to enter into the mouth of a human and be orally consumed or ingested, for example, a drink or shake. A beverage may be in a ready-to-drink liquid form (may be consumed without modification) or in a liquid, solid or other concentrate form, which can be transformed into a ready-to-drink liquid form with an addition of water or another liquid. A beverage, when in a ready-to-drink liquid form, generally contains water.

The phrase “calorie” as used herein means a unit representing the energy provided by food. Carbohydrate, protein, fat and alcohol provide calories in the diet. Generally, carbohydrate and protein have about 4 calories per gram, fat has about 9 calories per gram and alcohol has about 7 calories per gram.

The phrase “carbohydrate blend” as used herein means a mixture or other combination of two or more carbohydrates.

The phrase “cardiovascular disease” as used herein means disease of the heart and/or blood vessels (arteries, veins and/or capillaries), for example, high blood pressure, a stroke, an aneurysm, a heart attack, coronary artery disease or coronary heart disease. A lipid profile is a conventional blood test that measures total cholesterol, triglycerides and HDL cholesterol. LDL cholesterol is then calculated from the results. A lipid profile is one measure of a human's risk of cardiovascular disease.

The term “chocolate” as used herein, for example, in connection with one or more components of nutritional supplements, includes, but is not limited to, white chocolate, dark chocolate, light chocolate, milk chocolate, semi-sweet chocolate, bittersweet chocolate, extra-bittersweet chocolate, sugar-free chocolate, unsweetened chocolate, German chocolate, German sweet chocolate, coverture, imitation chocolate, or other types of chocolate, or any combination thereof. Such types of chocolate are commercially available from sources known by those of skill in the art, or may be produced in accordance with methods known by those of skill in the art.

The term “component” as used herein means a part, portion, element, constituent or ingredient, and is used interchangeably with “ingredient.” For example, in connection with a nutritional supplement, this term means an ingredient, or combination of ingredients, used to prepare the nutritional supplement, or a part, portion, element or constituent thereof, depending upon the context in which this term is used, which may readily be determined by those of skill in the art.

The term “concentrate” as used herein means a liquid or solid form of a liquid that has had the majority of its base component, or solvent, such as water, removed therefrom. Typically, this will involve the removal of water from the liquid. Concentrates include liquid concentrates and solid concentrates.

The phrase “Daily Reference Value” (DRV) as used herein means a label reference value set by the U.S. Food and Drug Administration for use in declaring the nutrient content of a food product (solid or liquid) on its label or labeling.

The phrase “Daily Value” (DV) as used herein means a value set by the U.S. Food and Drug Administration (FDA) for assisting consumers in understanding the relative significance of information about the amount of certain nutrients, such as vitamins and minerals, in a food product in the context of a total daily diet. This value assists consumers in comparing the nutritional values of food products. The Daily Value is determined by the FDA by combining the Reference Daily Intakes (RDIs) and the Daily Reference Values (RDAs) label reference values. The term “Daily Value,” thus, refers to the combined set of label reference values.

The phrase “diluent” as used herein means a substance or agent that dilutes, facilitates a physical separation of one or more ingredients and/or makes thinner or weaker, and preferably is water-soluble. Diluent materials that are suitable for human consumption could generally include complex polysaccharides, carbohydrates, smaller sugars (dextrose, sucrose and the like), dicalcium phosphate, tricalcium phosphate, maltodextrin and water.

The term “edible” as used herein means capable of being eaten, consumed and/or ingested by a mammal without being poisonous or otherwise harmful to a human.

The phrase “edible fats and oils” as used herein mean fats and/or oils that are edible, and that may be employed in food items and/or food products, such as nutritional supplements. Edible fats and oils include, but are not limited to, saturated fats, unsaturated fats, polyunsaturated fats, monounsaturated fats, trans fat, fatty acids, animal fats, vegetable fats, dairy fats, marine oils, plant oils (including vegetable oils, plant seed oils, nut oils and fruit oils), algae oils, microalgae oils, fungal oils, minerals oils, synthetic oils or fats and/or oils derived or obtained from other sources, components thereof and/or combinations thereof, for example, Evening Primrose oil, Black Currant seed oil, Borage oil, Borage seed oil, safflower oil, safflower seed oil, sunflower oil, sunflower seed oil, sesame seed oil, peanut oil, walnut oil, almond oil, olive oil, olive seed oil, avocado oil, avocado seed oil, pumpkin seed oil, corn oil, cod liver oil, soy oil, soybean oil, coconut oil, palm oil, palm kernel oil, rapeseed oil, flaxseed (linseed) oil, cotton seed oil, tung oil, palmolein oil, mustard seed oil, oiticica oil and castor oil. Other edible fats and/or oils are known by those of skill in the art. A wide variety of edible fats and oils are commercially available from sources known by those of skill in the art.

The phrase “edible organic acid” as used herein means an organic acid that is edible, such as citric acid, ascorbic acid, malonic acid, malic acid, adipic acid, tartaric acid and other edible carboxylic acids, or combinations thereof.

The term “emulsifier” as used herein means any substance or agent that aids in the formation of an emulsion. Edible emulsifiers include, for example, egg yolk, egg lecithin, soy lecithin and mono- and di-glycerides.

The term “emulsion” as used herein means a generally stable and homogeneous mixture of two liquids that do not normally mix (i.e., they are immiscible between themselves), such as vegetable oil and water, for example, milk and mayonnaise. Emulsions can be true colloids or less stable mixtures, such as salad dressing, which tend to separate in a short time. An emulsion can often be broken down (i.e. the liquids separated) by factors such as mechanical manipulation, chemical effects and/or time.

As used herein, the phrase “enteral administration” includes tube feeding (attaching a tube to a human that permits a nutritional product to be delivered to the gastrointestinal tract of the human without entering into the mouth) and parenteral administration (supplying nutrition directly to the blood system of a human, for example, intravenously). Tube feeding or parenteral administration may be employed to provide nutrition to humans that are unconscious, in a coma, have a severe facial injury, have Alzheimer's disease, have other dementia or otherwise experience difficulty swallowing (dysphasia) or consuming food. Tubes that transport nutritional formulas can be inserted, for example, into the stomach (G-tubes), through the nose and into the stomach (NG-tubes), or through the nose and into the small intestine (NJ tubes).

The phrase “essential amino acids” as used herein means amino acids that are necessary nutrients, but that are not synthesized by the body and, thus, need to be supplied as part of the diet.

The phrase “essential fatty acids” as used herein means fatty acids that are necessary nutrients, but that are not synthesized by the body and, thus, need to be supplied as part of the diet. Essential fatty acids include, for example, linoleic, linolenic and arachidonic acids.

The phrase “essential nutrient” as used herein means a nutrient that has a National Academy of Science RDA and/or RDI.

The term “fat” as used herein means any of the various saturated and/or unsaturated (including monounsaturated and polyunsaturated), hydrogenated or unhydrogenated soft solid, semisolid and/or solid organic compounds that generally comprise the glyceride esters of fatty acids and associated phosphatides, sterols, alcohols, hydrocarbons, ketones and/or related compounds, components thereof and/or mixtures or other combinations thereof. Such components include, but are not limited to, fatty acids, glycerides (mono-, di- and tri-), ethyl and other esters of fatty acids, as well as components thereof, and combinations thereof. Fats occur widely in organic tissue, particularly in the subcutaneous connective tissue of animals (beef, poultry, pork, lamb, liver and the like), and in the seeds, nuts and fruits of plants. Fats may be obtained or derived from animal, marine, plant, dairy, fungal or other origins. There is generally no chemical difference between fats and oils, with the only distinction being that fats are generally solid at room temperature and oils are generally liquid at room temperature. Examples of fats include butterfat, butter, cocoa butter, margarine, lard, tallow, milk fat, cream fat, fat from a wide variety of cheeses and the like. A wide variety of fats are known, and are commercially available from sources known by those of skill in the art.

The phrase “fat blend” as used herein means a mixture or other combination of two or more fats and/or oils.

The phrase “fatty acids” as used herein means carboxylic acids that generally are derived from, or contained in, an animal, vegetable or other fat or oil, whether saturated, unsaturated, monounsaturated, polyunsaturated, aromatic, essential, nonessential, in a cis or trans form, in the ethyl esters, mono-, di- or tri-glycerides, free fatty acids or other forms, and components and combinations of the foregoing. Fatty acids include, but are not limited to, omega-3 fatty acids, omega-6 fatty acids and omega-9 fatty acids, and the specific fatty acids identified below:

| | Number of | Number of | |
|---|---|---|---|
| | Carbon | Double | Fat or Oil |
| Common Name | Atoms | Bonds | Source |
| Butyric Acid | 4 | 0 | Butterfat |
| Caproic Acid | 6 | 0 | Butterfat |
| Caprylic Acid | 8 | 0 | Coconut Oil |
| Capric Acid | 10 | 0 | Coconut Oil |
| Lauric Acid | 12 | 0 | Coconut Oil |
| Myristic Acid | 14 | 0 | Palm Kernel Oil |
| Palmitic Acid | 16 | 0 | Palm Oil |
| Palmitoleic Acid | 16 | 1 | Animal Fats |
| Stearic Acid | 18 | 0 | Animal Fats |
| Oleic Acid | 18 | 1 | Olive Oil |
| Linoleic Acid | 18 | 2 | Corn Oil |
| Alpha-Linolenic Acid | 18 | 3 | Flaxseed |
| (ALA) | | | (Linseed) Oil |
| Gamma-Linolenic Acid | 18 | 3 | Borage Oil |
| (GLA) | | | |
| Arachidic Acid | 20 | 0 | Peanut Oil, |
| | | | Fish Oil |
| Gadoleic Acid | 20 | 1 | Fish Oil |
| Arachidonic Acid (AA) | 20 | 4 | Liver Fats |
| Eicosapentaenoic Acid | 20 | 5 | Fish Oil |
| (EPA) | | | |
| Behenic Acid | 22 | 0 | Rapeseed Oil |
| Erucic Acid | 22 | 1 | Rapeseed Oil |
| Docosahexaenoic Acid | 22 | 6 | Algae Oil or |
| (DHA) | | | Fungal Oil |
| Lignoceric Acid | 24 | 0 | Most Fats |

Other fatty acids are known by those of skill in the art. A wide variety of fatty acids are commercially available from sources known by those of skill in the art. Also, edible oils can be separated into their component fatty acids on a capillary column in a gas chromatograph, and the relative fatty acid contents measures. Additional information concerning fatty acids is readily available from the Fatty Acid Producer's Council (New York, N.Y.).

The term “fructan” as used herein means a class of materials that include inulin, oligofructose and fructooligosaccharide (FOS).

The phrase “fully cold-formed” as used herein means that a food product, such as a food bar, is produced without the use of any heat.

The phrase “fungal oil” as used herein means an oil that is derived or obtained from a fungal source, whether modified or unmodified, such as Mucor javanicus, either directly or indirectly. As used herein, the phrase “fungal oil” includes, but is not limited to, one or more individual components present in fungal oil, such as DHA, arachidonic acid or other fatty acids. A wide variety of fungal oils are commercially available from known sources, such as Martek Corp. (Columbia, Md.).

The phrase “geriatric” as used herein in connection with human beings means a human being that generally ranges in age from about 60 years and above, and more usually from about 75 years and above.

The phrase “glycemia” as used herein means the presence of glucose in the blood.

The phrase “harmful” as used herein means harmful to humans according to one or more established standards, such as FDA or EPA guidelines or rules.

The phrase “hazardous heavy metals” as used herein includes heavy metals that are hazardous, including mercury, lead, uranium and arsenic.

The phrase “hermetically seal” as used herein in connection with a nutritional beverage means to seal or insulate (preferably completely) the nutritional beverage away from air (in a manner that it is prevented from coming into contact with air).

The phrases “high quality” and “high grade” as used herein in connection with DHA means that the DHA is identical, or equivalent, to the DHA molecule that is present in human breast milk (preformed). In contrast with DHA (or other omega-3 fatty acids) obtained, or derived, from aquatic animals, such high grade DHA (or other omega-3 fatty acids) generally does not have EPA associated, or otherwise present, with it, and consistently contains no detectable contaminants of public health significance for long-term consumption (mercury, chlorinated hydrocarbons, pesticides and the like). The stabilizing agents used as a carrier for the DHA generally are triglyceride based (human molecules) with human vitamin anti-oxidants. The purity and potency of the DHA may be of a sufficient grade for pharmaceutical use. High grade DHA can readily be tested using HPLC and/or AA (or other methodology) to confirm its specific DHA content, precise carrier content and contaminant levels, and to confirm an absence of one or more substances that are not naturally found in human DHA.

The term “hyperglycemia” as used herein means a blood glucose level that is above the normal range (high blood sugar).

The term “hypoglycemia” as used herein means a blood glucose level that is below the normal range (low blood sugar).

The term “humans” as used herein, unless otherwise stated, includes human beings that are babies, infants, children or adults.

The term “hydrogenation” as used herein means a chemical process by which hydrogen is added to unsaturated fatty acids to produce partially or fully hydrogenated oils. Hydrogenation converts unsaturated bonds in the oil into saturated bonds, creating a solid, spreadable fat. Some of the double bonds may be eliminated, while others may be incompletely transformed. These double bonds may be transformed from the natural “cis” configuration to the “trans” configuration. For example, vegetable shortening is created by the complete hydrogenation of vegetable oil.

The phrase “imitation chocolate” as used herein refers to a wide variety of chocolate-type products that generally do not contain cocoa butter (the fat that occurs naturally in cocoa beans), and have the cocoa butter replaced with one or more other fats and/or oils, such as soy oil or canola oil.

The term “ingredient” is used herein interchangeably with “component” in connection with nutritional supplements of the invention.

The phrase “liquid” as used herein means a fluid or semi-fluid, the shape of which is generally determined by the container that it fills.

The phrase “marine oil” as used herein has the meaning described hereinabove. As used herein, the phrase “marine oil” includes, but is not limited to, one or more individual components that are derived, or obtained, from marine oil, such as one or more omega-3 fatty acids, including DHA, EPA, ALA, or a combination thereof, that is derived, or obtained, from fish oil.

The term “medicament” as used herein means a substance or agent that promotes a recovery from an injury, illness, disease or disorder.

The term “mg/dL” as used herein means milligrams per deciliter, and is a unit of measure that shows the concentration of a substance in a specific amount of fluid. In the United States, blood glucose test results are reported as mg/dL. Medical journals and other countries use millimoles per liter (mmol/L). To convert to mg/dL from mmol/L, mmol/L is multiplied by 18. Example: 10 mmol/L×18=180 mg/dL.

The term “minerals” as used herein means minerals that are edible, and includes those in an elemental, salt or other form. Examples of minerals include, but are not limited to, calcium, copper, fluorine, iodine, iron, magnesium, manganese, molybdenum, potassium, phosphorous, selenium and zinc, in an elemental form, or in the form of carbonates, oxides, phosphates, silicates, sulfates, sulfides or other forms. Many minerals are inorganic compounds that are necessary for life and good nutrition, such as calcium, copper, iron, magnesium, potassium and zinc. Additional information about minerals, including an extensive list of minerals, is present at the web site http://en.wikipedia.org/wiki/Minerals.

The term “nutrient” as used herein means an agent or substance that is necessary for, or enhances, the growth, development and/or health of a mammal, and includes medicaments. Examples of nutrients include, but are not limited to, DHA and GLA.

The term “oil” as used herein means a fat that generally is viscous, liquid or liquefiable at room temperature, and includes mixtures and other combinations of one or more oils and/or components of oils, such as fatty acids, glycerides and/or ethyl esters of fatty acids (or components thereof). Oils may be derived or obtained from animal, marine, algae, microalgae, fungal, mineral, plant (including vegetables and plant seeds), fruit, nut, synthetic or other sources, and are generally composed largely of glycerides of the fatty acids, particularly oleic, palmitic, stearic and linolenic. Oils may be hydrogenated or non-hydrogenated, and saturated or unsaturated (including monounsaturated and polyunsaturated). Plant sources of oil include, but are not limited to, hydrogenated and non-hydrogenated vegetable oils and plant seed oils, Evening Primrose oil, Black Currant seed oil, Borage oil, Borage seed oil, safflower oil, safflower seed oil, sunflower oil, sunflower seed oil, sesame seed oil, peanut oil, olive oil, olive seed oil, corn oil, avocado oil, avocado seed oil, pumpkin seed oil, soy oil, soybean oil, coconut oil, palm oil, palm kernel oil, rapeseed oil, flaxseed (linseed) oil, cotton seed oil, tung oil, oiticica oil and castor oil. Hydrogenated vegetable oils are often referred to as “shortenings.” Examples of fats derived from marine sources include fish oil and algae oil. Other oils are known by those of skill in the art. A wide variety of oils are commercially available from sources known by those of skill in the art.

The phrase “palatable” as used herein means acceptable or pleasing to the taste (being sufficiently agreeable in flavor to be ingested or consumed).

The phrase “palatability enhancer” as used herein means an ingredient present in a food item or food product, such as a nutritional supplement, for example, a sweetener or flavoring agent, that is employed for the purpose of improving the taste of the food item or food product.

The phrase “partially cold-formed” as used herein means that a food product is produced using one or more heating steps, such as using a jacketed kettle, but without the use of any baking steps.

The phrase “Percent Daily Values” as used herein are values calculated as follows, which values are present in the guidelines of the National Academy of Sciences and Institute of Medicine (1968 or 1998-2001), and are based upon a 2,000 calorie diet:

$$Percent Daily Values = \frac{Recommended Dietary Allowances ( RDAs )}{Adequate Intakes ( AIs )}$$

The phrase “plant seed oil” as used herein means an oil that is extracted or otherwise obtained from a seed of a plant, either directly or indirectly, particularly oily seeds. As used herein, the phrase “plant seed oil” includes, but is not limited to, one or more individual components of plant seed oil, or mixtures thereof. Plant seed oils include, but are not limited to, Black Currant seed oil, Borage seed oil, safflower seed oil, sunflower seed oil, sesame seed oil, avocado seed oil, pumpkin seed oil, olive seed oil, coconut seed oil, rapeseed oil, flaxseed (linseed) oil, cottonseed oil and tung oil. Other plant seed oils are known by those of skill in the art.

The phrase “plant oil” as used herein means an oil that is extracted or otherwise obtained from a plant, either directly or indirectly, particularly oily plants, including one or more individual components thereof and mixtures thereof. Plant oils include, but are not limited to, Evening Primrose oil, Borage oil, safflower oil, sunflower oil, peanut oil, walnut oil, almond oil, avocado oil, olive oil, corn oil, soy oil, soybean oil, coconut oil, palm oil, palm kernel oil and castor oil. Other plant oils are known by those of skill in the art.

The phrase “pleasant taste” as used herein in connection with nutritional supplements of the invention means that the nutritional supplements have a taste that is pleasant, desirable and/or appealing to humans that consume the nutritional supplements and, thus, generally do not have “off-flavors” or a “rancid” or other undesirable or unappealing taste.

The phrase “pleasant smell” as used herein in connection with nutritional supplements of the invention means that, when smelled by a typical human, the nutritional supplements either have no detectable smell and/or odor, or have a smell and/or aroma that is pleasant, desirable and/or appealing and, thus, do not have a rancid or other undesirable or unappealing smell.

The term “plurality” as used herein means more than one, such as two, three, four, five, six, seven, eight, nine, ten, fifteen, twenty or the like.

The phrases “pre-diabetes” or “borderline diabetes” as used herein mean a condition in which blood glucose levels are higher than normal, but are not high enough for a diagnosis of diabetes. Humans that have pre-diabetes are at an increased risk for developing Type 2 diabetes, and for cardiovascular disease and stroke.

The phrase “premature” as used herein in connection with cardiovascular disease means that cardiovascular disease occurs in a human prior in time, or age, than such cardiovascular disease typically occurs in a human having similar characteristics (weight, health, traits, status and the like).

The phrase “premix” as used herein means a mixture that generally includes vitamins, minerals and/or diluent materials, and typically has previously been prepared. It is often preferable that a premix be completely soluble in an aqueous or lipid medium. In some cases, however, it is preferable that a dry premix work well in dry blending, rather than in a liquid medium. Liquid mediums can be water or fat soluble. However, to be completely soluble in a water medium, lipid components should be excluded from a premix.

The phrase “protein blend” as used herein means a mixture or other combination of two or more proteins and/or amino acids.

The term “rancid” as used herein means having a disagreeable or otherwise undesirable taste and/or odor as a result of a partial or complete oxidation, degradation and/or decomposition of one or more edible fats and/or oils, such as a sour, stale and/or rank taste or odor.

The phrase “ready-to-drink liquid” as used herein means a liquid or beverage that may be consumed, or otherwise administered, in its current form (without any modification).

The phrase “Reference Daily Intakes” (RDIs) as used herein means a label reference value set by the U.S. Food and Drug Administration (FDA) for use in declaring the nutrient content of a food on its label or labeling. The FDA has replaced label reference values created in 1973 that are known as “U.S. Recommended Daily Allowances” (U.S. RDAs) with RDIs.

The phrase “risk factor” as used herein means any factor, trait or characteristic that raises the chances of a human developing a disease, such as obesity.

The phrase “room temperature” as used herein means the temperature in a room, which generally ranges from about 15° C. to about 30° C. (from about 59° F. to about 86° F.), and more usually ranges from about 21° C. to about 23° C. (from about 70° F. to about 74° F.). The “ambient temperature” of a room is “room temperature.”

The phrase “safe for consumption” in connection with nutritional supplements of the invention means that the nutritional supplements, and the components contained therein, in reasonable quantities administered for reasonable periods of time (such as those quantities and periods of time described herein, or as otherwise recommended for a particular human by a physician or other skilled clinician), which may vary for different types of humans, do not cause, or present a reasonable risk of causing, harm, illness, disease, injury, disorder or deformity to a human, such as spontaneous bleeding or the various known health hazards that can be caused by consuming mercury or other hazardous or radioactive chemicals that may be present in or on aquatic animals, oils obtained, or derived from, aquatic animals, or components of the foregoing.

The term “safe” in connection with nutritional supplements of the invention and developing fetuses or breast-fed babies means that the nutritional supplements, and the components contained therein, do not cause, or present a reasonable risk of causing, harm, illness, disease, injury, disorder or deformity to developing fetuses or breast-fed babies.

The phrase “shelf life” as used herein means an ability of a food product to remain “fresh” (i.e. to not have, or develop, any off flavors or tastes, or other undesirable tastes and/or odors) under room temperature conditions. For example, a food product may lose some or all of its “freshness” when an edible fat or oil, or another food product ingredient, undergoes an oxidation, degradation and/or decomposition.

The phrase “solid food product” as used herein means a food product that has a shape (i.e. that is not liquid). A solid food product may be solid or semi-solid. Solid food products include, for example, soft caramel chew candy, food bars and the other food items and food products described herein that have a shape.

The phrase “structured lipid” as used herein means a synthetic triglyceride comprising oil and medium chain triglyceride oil on the same glycerol backbone.

The phrase “sugar alcohol” as used herein means a sweetener that produces a smaller rise in blood glucose than other carbohydrates. The calorie content of sugar alcohols is about 2 calories per gram. Sugar alcohols include, for example, erythritol, hydrogenated starch hydrolysates, isomalt, lactitol, maltitol, mannitol, sorbitol, and xylitol.

The term “ulcer” as used herein means a generally deep and open sore or break in the skin.

General Description and Utility

The present invention provides nutritional supplements having a unique combination of functional nutritional ingredients at levels that are effective in mitigating, or assisting in the mitigation of, typical symptoms and/or conditions of borderline diabetes, diabetes, and other glucose intolerance disorders, which may otherwise be life threatening. These nutritional supplements, which may be considered as a “medical food,” were designed to control, maintain and normalize blood glucose levels, blood lipid levels and weight maintenance. These nutritional supplements can provide consistent, safe, cost-effective, desirable and well-rounded nutritional support for borderline diabetic, diabetic, geriatric, pregnant, lactating or other humans, including humans that have, or are at risk for, cardiovascular disease or otherwise have glucose intolerance, in a diet-assisted attenuation of overt, frank or developing disease conditions, such as those attendant with postprandial glucose tolerance, cardiovascular disease, circulatory disorder, insulin resistance, retinal degeneration and obesity and, thereby, also support a healthier higher quality of life nutritionally.

The nutritional supplements of the invention advantageously can provide a consistent formulated nutritional product to a human, which can be employed, for example, as a meal replacement or snack, and that is particularly beneficial for borderline diabetic, diabetic and other glucose intolerant humans. Issues relating to an unregulated diet in borderline diabetic, diabetic and other glucose intolerant humans are well known, and can include serious, life-threatening consequences. The present invention provides beneficial nutrition for humans in a known matrix and, thus, provides borderline diabetic, diabetic and other glucose intolerant humans with a dietary vehicle to overcome dangers that are inherent with an unregulated diet while, at the same time, fostering a safe and efficacious long term nutritional management of such conditions.

The nutritional supplements of the invention can provide a short- or long-term nutritional-assisted mitigation of borderline diabetic, diabetic, glucose intolerance, cardiovascular and other health symptoms and conditions, as well as prophylactic nutritional benefits to a broader population in need of them, such as members of a geriatric population, and humans that require or desire to have a glycemic index reduction in a cardio-protective and anti-inflammatory nutritional base (cardiovascular and pre diabetic conditions). In connection with symptoms and conditions that are commonly attendant with borderline diabetes, diabetes, glucose intolerance, cardiovascular disease and other adverse health conditions, nutritional supplements within the invention can be beneficial in improving a mammal's health and quality of life, as also reduce healthcare costs.

Additionally, these nutritional supplements should aid in preventing or delaying problems, symptoms and/or conditions that may be associated with, or result from, a pre-diabetic situation becoming a diabetic situation, and in preventing cardiovascular incidents. The general caloric and macronutrient profiles of the nutritional supplements of the invention, and the cardio-protective and anti-inflammatory benefits that they can provide to humans, render these nutritional supplements useful as a dietary food in the treatment for pre-diabetic conditions (i.e., to prevent such conditions from becoming realized into a full blown diabetic disease state). Preventing a condition, such as a pre-diabetic condition, from becoming more severe significantly reduces health care costs in addition to improving quality of life.

The nutritional supplements of the invention preferably include beneficial levels of all of the essential nutrients, as well as low quantities of fat, saturated fat, trans fat, cholesterol, carbohydrates and calories, controlled quantities of glucose and the levels of omega-3 fatty acids, such as DHA, folic acid, chromium, L-arginine, L-carnitine, lutein and the other vitamins, minerals, elements, nutrients and ingredients described herein.

The nutritional supplements of the invention can be employed as a dietary intervention adjunct to professional medical treatment for borderline diabetes, diabetes, other glucose intolerance disorders, cardiovascular disease and other illnesses, the treatment course of which may include a recommendation or need for a food product containing one or more omega-3 fatty acids, such as high quality DHA, low fat, low saturated fat, no trans fatty acids, low carbohydrate, low sodium, high folic acid and anti-oxidant content and/or a significant L-arginine and/or L-carnitine content.

The nutritional supplements of the invention include a unique combination of bioactive nutrients that, at effective levels, act synergistically, to attenuate, or aid in the attenuation of, various symptoms and/or conditions resulting from, or related to, borderline diabetes, diabetes and other glucose intolerance disorders, including cardiovascular symptoms, such as high blood pressure, circulatory symptoms, such as low HDL cholesterol, and anti-inflammatory symptoms, such as inflammation of blood vessels. Such nutrients, and levels thereof, generally are not provided by a typical mammalian diet.

The nutritional supplements of the invention comprise a nutritionally balanced formulation of energy, fat, carbohydrate and protein that minimizes the risk of chronic diseases that are more commonly found in diabetics and other glucose intolerant humans, as well as certain nutrients that are associated with increasing insulin sensitivity.

The cardio-protective and anti-inflammatory properties of the nutritional supplements of the invention render them useful in the dietary prevention, maintenance, management and/or control of symptoms and/or conditions resulting from, or related to, borderline diabetes, diabetes, other glucose intolerance disorders, cardiovascular disease and adverse symptoms experienced by geriatric humans, such as poor circulation, renal difficulties and degenerative eye disease. Additionally, these nutritional supplements may be employed by humans that are in a normal or satisfactory health that desire or need to consume nutritional supplements or meal replacements compositions that have the beneficial characteristics and properties of the nutritional supplements of the invention, such as being low in fats and carbohydrates.

In one serving thereof, nutritional supplements within the invention can provide important dietary components for the control, management and/or normalization of blood glucose levels, borderline diabetes, diabetes, other glucose intolerance disorders, cardiovascular disease and/or adverse symptoms and/or conditions experienced by geriatric patients, such as poor peripheral extremity circulation. One serving can also provide a level of lipids that are considered to be desirable for the control, management and/or maintenance of weight, which is often very important for borderline diabetic or diabetic humans.

The nutritional supplements of the invention may be used as a dietary meal replacement and/or supplement to provide beneficial short- and/or long-term nutritional maintenance and support to borderline diabetic, diabetic, glucose intolerant, geriatric and other humans, including humans that have, or are at risk for, cardiovascular symptoms or conditions, or as a beneficial nutritional supplement for use by the general population, for example, in the home, in in-home ambulatory treatment channels or facilities, in pharmacies and/or in healthcare facilities, such as medical clinics or offices, hospitals, nursing homes or hospice facilities.

### ADVANTAGES OF THE INVENTION

In addition to other advantages described herein, which may result from the use in the nutritional supplements of a particular ingredient, or amount thereof, the nutritional supplements of the invention generally have the advantages described below, which are very beneficial, and which result in nutritional supplements that are improved in comparison with known or other nutritional supplements.

### Palatability, Smell and Bioavailability

The nutritional supplements of the invention generally have a pleasant taste and/or a pleasant smell (generally both). Such taste and smell has an effect of encouraging consumption of the nutritional supplements by diabetic, borderline diabetic, otherwise glucose intolerant, geriatric or other humans, including humans that have, or are at risk for, cardiovascular disease, or that are in a normal health.

Nutritional supplements within the invention overcome shortcomings of many other nutritional supplements, such as large vitamin pills, and can deliver to humans specific doses of macronutrients, vitamins, minerals and/or other nutritional agents in a bioavailable absorption format. The dispersed system of nutritional supplements within the invention diminishes concerns related to the disintegration and/or dissolution of classical solid dosage forms, and the bioavailability of some nutritional supplements contained therein.

Because nutritional supplements within the invention are not in the form of large pills, tablets or capsules, and have a pleasant taste and/or smell, these nutritional supplements provide for an improved patient compliance with the daily regime of consuming vitamin, mineral and nutrient supplements during an often-difficult period of time.

### Macronutrient Profile

The nutritional supplements of the invention preferably are low in calories, and contain a unique macronutrient profile that is particularly beneficial for borderline diabetic, diabetic, other glucose intolerant and geriatric humans, humans that may have, are at risk for, or develop cardiovascular disease, and humans that desire or need to enhance their consumption of nutrients, or quantities thereof, or to lose weight. At the same time, these nutritional supplements are safe for consumption by humans, including those that may also be pregnant, lactating or having childbearing potential and are attempting to become pregnant, and are safe for their developing fetuses and/or breast-fed babies.

Nutritional supplements within the invention provide a dietary vehicle for managing, controling and normalizing blood glucose levels by slowly providing the human body with low levels of desirable or necessary carbohydrates, which are preferably in the form of a carbohydrate blend including one or more slow-digesting carbohydrates.

The nutritional supplements preferably also include a high grade DHA that is obtained from a source other than an aquatic animal, as well as L-arginine and one or more antioxidant agents, all of which are beneficial in assisting in the maintenance of an optimal cardiovascular function, as well as circulatory order, thus, helping to avoid complications, such as premature cardiovascular disease.

Additionally, the nutritional supplements may, optionally, include other nutrients, such as vitamins and/or minierals, preferably in an amount that ranges from at least about 1 to about 100% of the U.S. RDI therefore and, in some cases, more preferably in an amount that is at least about 100% of the U.S. RDI therefore.

Further, the nutritional supplements of the invention include a cardioprotective lipid base containing low levels of trans fatty acids. Preferably, the lipid base is a monounsaturated base that contains one or more omega-3 fatty acids. Such a lipid base generally has an effect of supporting health in humans that have, are at risk for, or develop, borderline diabetes, diabetes, other glucose intolerance disorders or cardiovascular disease.

Inflammation (as well as excessive weight) is an important risk factor for transforming pre-diabetes into diabetes. Ingredients that may be included in nutritional supplements within the invention, such as DHA (and/or other omega-3 fatty acids), L-arginine, monosaturates and anti-oxidants, as well as the low level of calories that are present in therein, each can be effective in reducing an inflammation in a human and, thus, render the nutritional supplements beneficial in mitigating, or assisting in the mitigation of, pre-diabetes (i.e., preventing or delaying pre-diabetes from transforming into diabetes or another glucose intolerance disorder).

### Aquatic Animal Sources of Ingredients

Because components obtained, or derived, from aquatic animals, such as fish, fish oil, and components of fish oil, may contain mercury or other toxic or radioactive chemicals, which can present serious health hazards for humans, particularly pregnant women, lactating women and women having childbearing potential that are attempting to become pregnant, and their developing fetuses or breast-fed babies, the nutritional supplements of the invention may, but preferably do not, contain detectable amounts of any oils (or components thereof), or other components, that are obtained, or derived, from aquatic animals, and more preferably do not contain any amount of these oils or components. Methods for detecting such oils and components include known high performance liquid chromatographic (HPLC) methods.

### Safe for Consumption

The nutritional supplements of the invention were designed and formulated so that they would be safe for consumption by humans, including diabetic, borderline diabetic and geriatric humans, and humans that have, are at risk for, or develop other glucose intolerance or cardiovascular disease (and preferably also for pregnant or lactating women, and women having childbearing potential that are attemptiong to become pregnant, and also safe for developing fetuses and/or breast-fed babies). Preferably, all components included in the nutritional supplements have a beneficial nutritional, medical or other health benefit for humans, and none of the components present in the nutritional supplements would present a health hazard or other risk to any human (including breast-fed babies), or to a developing fetus.

### Easy to Digest

The nutritional supplements of the invention generally are easy to digest by virtue of preferable thermal processing, which helps hydrolyze the protein component, and an absence of difficult-to-digest components (which are preferably excluded from the nutritional supplements).

### Shelf Life

The nutritional supplements of the invention generally remain “fresh” (continue to have a pleasant taste and/or smell, and a stability against oxidative rancidity) for a period of at least about one year under room temperature conditions and, in some cases, possibly even about two years under these conditions. These nutritional supplements generally have a shelf life of at least about one year under these conditions in connection with flavor and taste alone and, possibly, significantly longer in connection with nutrient compliance.

### Use in Weight Loss Programs

The nutritional supplements could be employed as part of a prescribed weight loss program for virtually the entire population (with the exception of infants).

Ingredients and Nutrition Facts

The nutritional supplements of the invention may include the ingredients, and amounts thereof, described below, and may contain additional and/or other ingredients.

### Vitamin A (Palmitate)

Vitamin A refers to a group of fat-soluble substances that are structurally related to, and possess, the biological activity of the parent substance of the group called all-trans retinol or retinol. Vitamin A occurs naturally in the form of fatty acid esters, such as vitamin A palmitate (retinyl palmitate). Vitamin A plays vital roles in vision, epithelial differentiation, growth, reproduction, pattern formation during embryogenesis, bone development, hematopoiesis and brain development. It generally prevents night blindness and inflammation or dryness of the eyes, and also is important for the maintenance of the proper functioning of the immune system. Certain carotenoids, such as beta-carotene alpha-carotene and beta-cryptoxanthin, are dietary precursors of vitamin A. Collectively, these substances are called provitamin A. Beta-carotene is also an antioxidant that can provide, or enhance, a stability to fats and/or oils, and to food or nutritional products including fats and/or oils, and that provides benefits of antioxidants to the human body.

Vitamin A may, optionally, be included in the nutritional supplements of the invention. However, because high levels of pre-formed vitamin A (palmitate) have been potentially linked to birth defects, it is preferred that any vitamin A employed in the nutritional supplements be present in the form of beta-carotene or another precursor of vitamin A alone or in combination, or beta-carotene in combination with a lower level of palmitate or vitamin A (palmitate). Those of skill in the art know, or can readily determine, various ratios of such substances that can safely be employed in foods or nutritional supplements.

The amount of vitamin A palmitate that may, optionally, be present in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for supplying essential vitamin A nutrition, in combination with pro vitamin A (beta-carotene), which is important for vision, bone growth, reproduction, cell growth and helps with regulation of the immune system in aiding in the prevention of infections. This amount may vary depending upon the type, age, health and status of the humans that are to consume the nutritional supplements, and like considerations, but generally ranges from about 400 IU beta-carotene and about 800 IU vitamin A as palmitate to about 1000 IU beta carotene and about 2000 IU vitamin A as palmitate per day, and preferably ranges from about 600 IU beta carotene and about 1000 IU vitamin A as palmitate to about 750 IU beta-carotene and about 1400 IU vitamin A as palmitate per day, with about 700 IU beta carotene and about 1200 IU vitamin A as palmitate per day being most preferred.

The nutritional supplements of the invention preferably contain a lower concentration of vitamin A palmitate than is present in known nutritional beverages, such as Glucerna. Such a lower concentration of vitamin A palmitate renders the nutritional beverages safer for consumption by pregnant or lactating women, or women having childbearing potential that are attempting to become pregnant, that are diabetic or borderline diabetic, or that have cardiovascular disease (and for their fetuses or breast-fed babies).

The amount of vitamin A that may, optionally, be included in the nutritional supplements of the invention may vary widely, in the same manner described above for vitamin A palimtate, and to produce the same or similar effects, but generally ranges from about 1,000 to about 8,000 IU per day, and preferably ranges from about 1,500 IU to about 5,000 IU per day.

Preferably, a daily quantity of the nutritional supplements of the invention includes at least about 100% U.S. RDI for vitamin A, and one 8-fluid ounce serving of a ready-to-drink liquid nutritional supplement of the invention includes about 1750 IU of vitamin A. More preferably, such an 8-fluid ounce serving contains Vitamin A as about 660 IU beta-carotene and about 1090 IU vitamin A as palmitate.

### Vitamin C

Vitamin C (C₆H₈O₆) is the L-enantiomer of ascorbic acid, and is a water-soluble vitamin used by the body for several purposes. Ascorbic acid is an organic acid and antioxidant. Cardiovascular diseases, cancers, joint diseases and cataracts are all associated with vitamin C deficiency, and can be partly prevented by optimal intake of vitamin C. Vitamin C achieves much of its protective effect by functioning as an antioxidant and preventing oxygen-based damage to cells.

The nutritional supplements of the invention may, optionally, include vitamin C in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for providing, or enhancing, an antioxidant protection of DNA, membrane lipids and/or proteins, enhancing, or maintaining, the health of teeth, gums and/or bones, and/or immune function or collagen synthesis, promoting the healing of wounds and/or scar tissue, preventing, or aiding in the prevention of, scurvy, aiding in the prevention of cardiovascular diseases, cancers, joint diseases, cataracts and/or oxygen-based damage to cells, and/or preventing, or decreasing, an oxidation, degradation and/or decomposition of one or more fats and/or oils that are present in the nutritional supplements. This amount may vary depending upon the type, age, health and status of the humans that are to consume the nutritional supplements, and like considerations, but generally ranges from about 30 mg to about 500 mg per day, and preferably range from about 40 mg to about 180 mg per day.

Preferably, a daily quantity of the nutritional supplements of the invention includes at least about 100% U.S. RDI for vitamin C, and one 8-fluid ounce serving of a ready-to-drink liquid nutritional supplement of the invention includes about 60 mg of vitamin C.

### Vitamin E

Vitamin E (tocopherol) is a fat-soluble vitamin vitamin that is an important antioxidant, and that exists in eight different forms, each of which has its own biological activity. Alpha-tocopherol is the most active form of vitamin E in humans, and is a powerful biological antioxidant. Antioxidants such as vitamin E act to protect cells against the effects of free radicals, which are potentially damaging by-products of the body's metabolism. Free radicals can cause cell damage that may contribute to the development of cardiovascular disease and cancer.

The nutritional supplements of the invention may, optionally, include vitamin E in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for protecting, or aiding in the protection of, cells against the effects of free radicals, preventing, or aiding in the prevention of, a development of cardiovascular disease and/or cancer and/or enhancing cardiovascular health. This amount may vary depending upon the type, age, health and status of the humans that are to consume the nutritional supplements, and like considerations, but generally ranges from about 10 IU to about 400 IU per day, and preferably ranges from about 20 IU to about 90 IU per day.

Preferably, a daily quantity of the nutritional supplements of the invention includes at least about 100% U.S. RDI for vitamin E, and one 8-fluid ounce serving of a ready-to-drink liquid nutritional supplement of the invention includes about 30 IU of vitamin E.

### Folic Acid

Folic acid (once called vitamin M) is a B-complex vitamin that prevents, or aids in the prevention of, defects in the neural tube (the embryonal structure that gives rise to the brain and spinal cord) in developing human fetus. The anion form of folic acid is called folate.

Defects in the neural tube of a developing fetus, such as spina bifida, can occur during the first trimester of pregnancy, for example, during the first month of gestation, before a woman may have become aware of her pregnancy. These defects are known to be linked to an inadequate intake of folic acid. Because folic acid is known to prevents neural tube defects, folic acid should be consumed in sufficient quantities by women of childbearing ages. Folic acid has also been shown to have beneficial cardiac effects, and to decrease the risk of cervical dysplasia.

Homocysteine is an amino acid in the blood. Too much homocysteine is related to a higher risk of coronary heart disease, stroke and peripheral vascular disease (fatty deposits in peripheral arteries). Evidence suggests that homocysteine may promote atherosclerosis (fatty deposits in blood vessels) by damaging the inner lining of arteries and promoting blood clots. Folic acid and other B vitamins help break down homocysteine in the body. Several studies have found that higher blood levels of B vitamins are related, at least in part, to lower concentrations of homocysteine. Other evidence shows that low blood levels of folic acid are linked with a higher risk of fatal coronary heart disease and stroke.

The nutritional supplements of the invention contain folic acid in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for maintaining, or enhancing, cardiovascular function and/or neurological function in humans, preventing, or reducing the severity of, neural tube defects of fetuses that are developing in pregnant women, enhancing a break down of homocysteine in the body and/or providing a benefit related to insulin sensitivity. This amount may vary depending upon the type, age, health and status of the humans that are to consume the nutritional supplements, and like considerations, but generally ranges from about 0.2 mg to about 1.0 mg per day, and preferably ranges from about 0.4 mg to about 0.8 mg per day.

Preferably, a daily quantity of the nutritional supplements of the invention includes at least about 100% U.S. RDI for folic acid, and one 8-fluid ounce serving of a ready-to-drink liquid nutritional supplement of the invention includes about 0.4 mg of folic acid. In contrast, Glucerna contains only 50% of the U.S. RDI for folic acid.

As a result of folic acid that may be present therein, the nutritional supplements of the invention may have beneficial properties of protecting against neural tube defects.

### Calcium

Calcium is a mineral that is important for proper bone and tooth formation, muscle contraction, weight loss, nerve transmission and blood clotting.

The nutritional supplements of the invention may, optionally, contain calcium in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for enhancing proper bone and/or tooth formation, muscle contraction, weight loss, nerve transmission and/or blood clotting. This amount may vary depending upon the type, age and health of the humans that are to consume the nutritional supplements, and like considerations, but generally ranges from about 200 mg to about 2,000 mg per day, and preferably ranges from about 500 mg to about 1,000 mg per day.

Preferably, a daily quantity of the nutritional supplements of the invention includes from about 25% to about 75% of the RDI for calcium, and one 8-fluid ounce serving of a ready-to-drink liquid nutritional supplement of the invention includes about 250 mg of calcium.

### Sodium and Potassium

The nutritional supplements of the invention may, optionally, include sodium and/or potassium, and preferably include low quantities of sodium and high quantities of potassium.

Sodium

Sodium aids in the regulation of blood pressure and blood volume, and can help in nerve and muscle function.

The amount of sodium that may be included in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for aiding in the regulation of blood pressure and/or blood volume, and/or in nerve and/or muscle function. This amount may vary depending upon the type, age, health and status of the humans that are to consume the nutritional supplements, and like considerations, but generally ranges from about 400 mg to about 2400 mg per day, and preferably ranges from about 1400 mg to about 2400 mg per day.

Preferably, a daily quantity of the nutritional supplements of the invention includes less than about 10% to about 30% of the DV for sodium, and one 8-fluid ounce serving of a ready-to-drink liquid nutritional supplement of the invention includes about 170 mg of sodium.

Potassium

Potassium aids in maintaining the electrical stability of the cells of the heart and nervous system, and is necessary for the building of muscle and normal body growth.

The amount of potassium that may be included in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for aiding in maintaining the electrical stability of the cells of the heart and nervous system, the building of muscle and/or normal body growth. This amount may vary depending upon the type, age, health and status of the humans that are to consume the nutritional supplements, and like considerations, but generally ranges from about 400 mg to about 4000 mg per day, and preferably ranges from about 500 mg to about 3500 mg per day.

Preferably, a daily quantity of the nutritional supplements of the invention includes from about 10% to about 30% of the DV for potassium, and one 8-fluid ounce serving of a ready-to-drink liquid nutritional supplement of the invention includes about 350 mg of potassium.

### Iron

Iron is necessary for the formation of hemoglobin, which carries oxygen from the lungs to the cells of the human body. Iron is also involved in the storage of oxygen, immune function, and the formation of new red blood cells. However, in large or excessive quantities, iron can have deleterious effects upon, or in connection with, the human body.

The nutritional supplements of the invention may, optionally, include iron. The amount of iron that may be included in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, is safe for the developing fetuses and/or breast-fed babies of pregnant or lactating women, does not have deleterious effects upon the human body, and that is effective for enhancing the formation of hemoglobin, the storage of oxygen, immune function and/or the formation of new red blood cells in the human body. This amount may vary depending upon the type, age, health and status of the humans that are to consume the nutritional supplements, and like considerations, but generally ranges from about 0% to about 75% of the minimum U.S. RDI for iron, and preferably ranges from about 15% to about 35% of the minimum U.S. RDI for iron.

Preferably, one 8-fluid ounce serving of a ready-to-drink liquid nutritional supplement of the invention includes about 4.5 mg of iron.

### Other Vitamins and Minerals

A wide variety of vitamins and minerals may be included in, or used to prepare, the nutritional supplements of the invention in varying quantities.

In addition to the vitamins and minerals that are otherwise described herein, other vitamins and/or minerals that may be included in the nutritional supplements of the invention include, for example, Vitamin B₁ (as Thiamine or Thiamine Mononitrate), Vitamin B₂ (as Riboflavin), Vitamin B₃ (as Niacin), Vitamin B₆ (as Pyridoxine or Pyridoxine Hydrochloride), Vitamin B₁₂ (Cyanocobalamin), Biotin, Vitamin D, Vitamin K, Folacin, Niacinamide, Iron (as Ferrous Fumarate), Phosphorus, Pantothenic Acid (as Calcium Pantothenate), Iodine (as Potassium Iodide), Magnesium (as Magnesium Oxide), Zinc (as Zinc Oxide), Selenium, Copper (as Cupric Oxide), Manganese (as Manganese Sulfate), Molybdenum, Choline, Fluoride, Chloride, Biotin and various mixtures or other combinations thereof. Vitamins and minerals are commercially available from sources known by those of skill in the art, such as Hoffmann-LaRoche Inc. (Nutley, N.J.).

Premixes containing vitamins and minerals recommended for pregnant women, lactating women and women having childbearing potential that are attempting to become pregnant (or other humans) that may be employed to produce nutritional supplements of the invention may be obtained from known sources, such as Watson Foods Co., Inc. (under Watson Code WT-6061A).

A vitamin and mineral premix that is particularly preferred for use in preparing the nutritional supplements of the invention (“Premix”) contains the nutrients and ingredients set forth below (per 2 g of Premix).

### Preferred Vitamin and Mineral Premix

| Nutrient | Per 6,000 mg | |
|---|---|---|
| Vitamin A (Vitamin A Palmitate) | 2500 | I.U. |
| Vitamin A (Beta Carotene) | 2500 | I.U. |
| Thiamin (Thiamin Mononitrate) | 3 | mg |
| Riboflavin | 3.4 | mg |
| Niacin (Niacinamide) | 20 | mg |
| Pyridoxine (Pyridoxine Hydrochloride) | 10 | mg |
| Folic Acid | 1 | mg |
| Vitamin B₁₂ (Cyanocobalamin) | 0.012 | mg |
| Biotin | 0.3 | mg |
| Vitamin C (Ascorbic Acid) | 120 | mg |
| Vitamin D (Cholecalciferol) | 400 | I.U. |
| Vitamin E (d, 1 Alpha Tocopherol Acetate) | 30 | I.U. |
| Calcium (Dicalcium Phosphate, Calcium Carbonate) | 1200 | mg |
| Iron (Ferrous Fumarate) | 27 | mg |
| Pantothenic Acid (d-Calcium Pantothenate) | 10 | mg |
| Iodine (Potassium Iodide) | 0.15 | mg |
| Magnesium (Magnesium Oxide) | 400 | mg |
| Zinc (Zinc Oxide) | 25 | mg |
| Selenium (Sodium Selenate) | 0.07 | mg |
| Copper (Cupric Oxide) | 2 | mg |
| Manganese (Manganese Sulfate) | 5 | mg |
| Chromium (Chromium Chloride) | 0.125 | mg |
| Molybdenum (Sodium Molybdate) | 0.0750 | mg |
| Maltodextrin, QS to make | 6,000 | mg |
| Component | Amount | |
| Vitamin A (as Betacarotene) | 660 | IU |
| Vitamin A (as Palmitate, USP-FCC) | 1090 | IU |
| Vitamin D3 (as Cholecalciferol, USP-FCC) | 100 | IU |
| Vitamin E (as Acetate, USP) | 30 | IU |
| Biotin (FCC) | 75 | mcg |
| Folic Acid (USP-FCC) | 0.4 | mg |
| Niacin (as Niacinamide, USP-FCC) | 5 | mg |
| Pantothenic Acid (as D-Calcium Pantothenate, USP) | 2.5 | mg |
| Vitamin B1 (as Thiamin HCl, USP-FCC) | 0.38 | mg |
| Vitamin B12 (as Cyanocobalamin, USP) | 3 | mcg |
| Vitamin B2 (as Riboflavin, USP-FCC) | 0.43 | mg |
| Vitamin B6 (as Pyridoxine HCl, USP-FCC) | 1 | mg |
| Vitamin C (as Ascorbic Acid, USP-FCC) | 60 | mg |
| Vitamin K1 (as Phytonadione, FCC) | 20 | mcg |
| Chloride (as Choline Chloride, USP-FCC) | 34 | mg |
| Chromium (as Chromium Chloride (6 H20), USP) | 0.12 | mg |
| Copper (as Copper Sulfate) | 0.5 | mg |
| Iodine (as Potassium Iodide, USP-FCC) | 38 | mcg |
| Iron (as Ferrous Sulfate, Dried, USP-FCC) | 4.5 | mg |
| Manganese (as Manganese Sulfate, USP-FCC) | 1 | mg |
| Molybdenum (as Sodium Molybdate) | 38 | mcg |
| Selenium (as Sodium Selenite) | 18 | mcg |
| Zinc (as Zinc Sulfate, USP-FCC) | 3.8 | mg |
| L-Arginine (USP) | 1,000 | mg |
| L-Carnitine (USP) | 31 | mg |
| Choline (as Choline Chloride, USP-FCC) | 100 | mg |
| Lutein (as Lutein, 5%) | 0.5 | mg |
| Meso-Inositol (FCC) | 186 | mg |

| | |
|---|---|
| Maltodextrin (as Maltrin M-100) | O.S. |

Several characteristics of the method for preparing the Premix are set forth below. However, methods for preparing other premixes may have different characteristics.

Dry-Blending Process

The Premix is a free-flowing powder product that is preferably prepared using a dry-blending, powder-to-powder process. Generally, during this process, a series of one or more powder ingredients or materials is added to and/or mixed with one or more other powder ingredients or materials. No water or other liquid is added to any ingredient or material used to prepare the Premix, or during any step of the method for preparing the Premix.

The water content of the Premix produced by this process preferably is less than about 5 weight percent. In this case, any water that may be present in the Premix is a result of water that occurs naturally in one or more ingredients or materials employed to prepare the Premix, such as maltodextrin (a diluent powder).

No spray-drying techniques or encapsulation techniques are employed in the method for preparing the Premix.

Carrier Material Powder

A carrier material powder is preferably employed in the method for preparing the Premix.

Diluent Power

A diluent powder, such as maltodextrin, is preferably employed in the method for preparing the Premix, generally for dilution purposes.

Complexes of Chromium and Carboxylic Acid

Chromium chloride, a chromium (III) salt, and ascorbic acid are employed in the method for preparing the Premix.

In order to form a complex of a chromium (III) salt and a carboxylic (or other) acid, such as ascorbic acid, the chromium (III) salt and the acid must be present together in a liquid solution (“solution”). The reaction between the chromium (III) salt and the acid to form a complex of the chromium (III) salt with the acid occurs in this solution.

When the Premix is prepared using a dry-blending, powder-to-powder process, a solution (aqueous or other) is never present, formed or otherwise employed during any of the steps of the method for preparing the Premix. Thus, there is no opportunity for a complex to be formed between the chromium chloride and ascorbic acid (or any other acid), and the Premix does not contain any complex between chromium and a carboxylic or other acid, such as a carboxylic acid complexed chromium (III) powder.

Further, the chromium chloride employed in the method for preparing the Premix preferably is not directly mixed with ascorbic acid (or any other edible organic acid) during any step of this method. Ascorbic acid is preferably added to other components present in the Premix at the end of the method for preparing the Premix (as the “last” step of the numerous steps employed in this method).

Citric Acid or Citric Acid Monohydrate

Neither citric acid (2-hydroxy-1,2,3-propanetricarboxylic acid), a carboxylic acid that is soluble in water, nor citric acid monohydrate, is present in the Premix, or is employed in the method for preparing the Premix.

Sodium Selenate

Sodium selenate is not present in the Premix, and is not employed in the method for preparing the Premix. Rather, sodium selenite is present in the Premix, and is employed in the method for preparing the Premix.

Hydrated Mineral Salts

Any hydrated mineral salts that may be employed during the method for preparing the Premix are added only during the last 20% to 30% of this method. (If one or more hydrated mineral salts was added during an earlier stage of this process, the hydrated mineral salts could cause a slush to form, which would disadvantageously inhibit or prevent the formation of the desired Premix product that is a free-flowing powder product.)

Nutritional supplements within the invention may be formulated using any pharmaceutically-acceptable forms of the vitamins and/or minerals described above, including their salts, which are known by those of skill in the art. For example, useful pharmaceutically-acceptable calcium compounds include any of the well-known calcium supplements, such as Calcium Carbonate, Calcium Sulfate, Calcium Oxide, Calcium Hydroxide, Calcium Apatite, Calcium Citrate-Malate, Bone Meal, Oyster Shell, Calcium Gluconate, Calcium Lactate, Calcium Phosphate, Calcium Levulinate, and the like. An instantly soluble calcium preparation that is composed of organic calcium salts, that is suitable for mineral fortification of food products, and that is known as Instant Calcium, is available from Flavor-Savor, Inc. (Franklin Park, Ill.). This product is generally odorless, tasteless and colorless when dissolved in either cold or hot water. Pharmaceutically acceptable magnesium compounds include Magnesium Stearate, Magnesium Carbonate, Magnesium, Oxide, Magnesium Hydroxide and Magnesium Sulfate. Pharmaceutically-acceptable iron compounds include any of the well-known Iron II (ferrous) or Iron III (ferric) supplements, such as Ferrous Sulfate, Ferric Chloride, Ferrous Gluconate, Ferrous Lactate, Ferrous Tartrate, Iron-Sugar-Carboxylate complexes, Ferrous Fumarate, Ferrous Succinate, Ferrous Glutamate, Ferrous Citrate, Ferrous. Pyrophosphate, Ferrous Cholinisocitrate, Ferrous Carbonate, and the like.

The vitamins and/or minerals that may be included in nutritional supplements of the invention may be microencapsulated in a coating of fat, microcrystalline cellulose or similar material in order to prevent their degradation under various conditions.

The vitamins and/or minerals that are preferred for use in the nutritional supplements of the invention are those that are essential or that have Daily Values (DVs), Reference Daily Intakes (RDIs) and/or Daily Reference Values (DRVs) described by the U.S. Food and Drug Administration (FDA) in its regulations or publications, such as the Code of Federal Regulations or Federal Register, Vol. 58, No. 3 (1993), for the following five categories of persons: (1) infants (persons not more than 12 months of age); (2) children under 4 years of age (persons 13 through 47 months of age); (3) adults and children that are 4 or more years of age; (4) pregnant women; and (5) lactating women.

One or more vitamins and/or minerals may be employed in the nutritional supplements in any quantity that is effective for providing, or enhancing, a nutritional, medical and/or other health benefit to a human, particularly a diabetic, borderline diabetic, otherwise glucose intolerant, geriatric, pregnant or lactating human, a human that has, is at risk for, or develops cardiovascular disease, a women having childbearing potential that is attempting to become pregnant or a developing fetus or breast-fed baby, and that is safe for consumption by the human, and is preferably safe for a developing fetus and/or breast-fed baby of the human. This quantity may vary widely depending upon the particular vitamins and/or minerals chosen for use, the age, size, weight and health condition of the human and like considerations, and preferably includes at least 100% of the U.S. RDI of DV for the vitamin or mineral. Low- or high-iron (as that term is defined in the art) food products may be prepared in accordance with the methods of the present invention.

The DVs described by the FDA, for example, in Federal Register, Vol. 58, No. 3 (1993) or in 21 CFR 101.9, for the different categories of human beings described above may be employed to determine such quantity for different humans, and are set forth below.

| | | | Children | Pregnant | Lactating | Persons that are |
|---|---|---|---|---|---|---|
| | Units | Infants | Under 4 Years | Women | Women | 4 Years or Older |
| Vitamin | | | | | | |
| Vitamin A | IU | 1,500 | 2,500 | 8,000 | 8,000 | 5,000 |
| Vitamin C | mg | 35 | 40 | 60 | 60 | 60 |
| Vitamin D | IU | 400 | 400 | 400 | 400 | 400 |
| Vitamin E | IU | 5 | 10 | 30 | 30 | 30 |
| Vitamin K | μg | *** | *** | *** | *** | 80 |
| Vitamin B₁ | mg | 0.5 | 0.7 | 2.5 | 2.5 | 1.5 |
| Vitamin B₂ | mg | 0.6 | 0.8 | 2.0 | 2.0 | 1.7 |
| Vitamin B₃ | mg | 8 | 9 | 20 | 20 | 20 |
| Vitamin B₆ | mg | 0.4 | 0.7 | 2.5 | 2.5 | 2.0 |
| Vitamin B₉ | mg | 1 | 0.2 | 0.8 | 0.8 | ** |
| Folate* | mg | *** | *** | *** | *** | 0.4 |
| Vitamin B₁₂ | μg | 2 | 3 | 8 | 8 | 6 |
| Biotin | mg | 0.05 | 15 | 0.3 | 0.3 | 0.3 |
| Mineral | | | | | | |
| Pantothenic | mg | 3 | 5 | 10 | 10 | 10 |
| Acid | | | | | | |
| Calcium | g | 0.6 | 0.8 | 1.3 | 1.3 | 1 |
| Phosphorus | g | 0.5 | 0.8 | 1.3 | 1.3 | 1 |
| Iodine | μg | 45 | 70 | 150 | 150 | 150 |
| Iron | mg | 15 | 10 | 18 | 18 | 18 |
| Magnesium | mg | 70 | 200 | 450 | 450 | 400 |
| Copper | mg | 0.6 | 1.0 | 2.0 | 2.0 | 2.0 |
| Zinc | mg | 5 | 8 | 15 | 15 | 15 |
| Selenium | μg | *** | *** | *** | *** | 70 |
| Manganese | mg | *** | *** | *** | *** | 2.0 |
| Chromium | μg | *** | *** | *** | *** | 120 |
| Molybdenum | μg | *** | *** | *** | *** | 75 |
| Chloride | mg | *** | *** | *** | *** | 3,400 |

*Folate is the anion form of folic acid (currently known as folacin or Vitamin B₉ and formerly known as vitamin M).
** Information not present in 21 CFR 101.9.
*** Information not present in Federal Register, Vol. 58, No. 3 (1993).

Additional information is present at the web site www.nal.usda.gov.

It is preferable that an amount of the nutritional supplements of the invention that is consumed in a 24-hour period, which may be divided into various quantities, such as one or more 8-ounce servings, provide at least 100% of the U.S. RDIs or DVs for all essential vitamins and minerals.

When employed with pregnant women, lactating women or women having childbearing potential that are attempting to become pregnant, it is important that the quantity of each vitamin and mineral used in a nutritional supplements of the invention be safe for consumption by such humans, and is safe for their developing fetuses and/or babies, or is safe for other humans that consume the food product. Larger quantities of certain vitamins or minerals may cause damage to a developing fetus or baby, or to other humans. It is also important that the quantity of each vitamin and mineral used in nutritional supplements of the invention be safe for consumption by other humans that consume such products. Those of skill in the art know the quantities of vitamins and minerals above the U.S. RDIs or DVs for pregnant women, lactating women, women having childbearing potential that are attempting to become pregnant, or for other humans, that would be harmful for the human consuming the nutritional supplement, or for their developing fetuses or babies.

Set forth hereinbelow are the approximate preferred ranges of the daily quantities of the various vitamins and minerals that may generally be used in nutritional supplements of the invention (or divided between more than one 8-fluid ounce of other serving of nutritional supplements in a ready-to-drink liquid form, or more than one 60-gram or other serving of nutritional supplements in a food bar or other solid form) for consumption during a one-day (24-hour) period for humans, including pregnant women, lactating women or women having childbearing potential that are attempting to become pregnant (from about one quantity to about another quantity), as well as more preferred ranges, and the most preferred quantities for pregnant and lactating women.

| | | | More | Most Preferred | Most Preferred |
|---|---|---|---|---|---|
| | | Preferred | Preferred | Quantity | Quantity for |
| | Units | Range | Range | for Pregnant Women | Lactating Women |
| Vitamin | | | | | |
| Vitamin A | IU | 0-9,000 | 1,500-8,000 | 8,000 | 8,000 |
| Vitamin C | mg | 0-1,000 | 35-500 | 60 | 60 |
| Vitamin D | IU | 0-800 | 200-400 | 400 | 400 |
| Vitamin E | IU | 0-1,500 | 5-400 | 30 | 30 |
| Vitamin K | μg | 0-80 | 10-80 | 10 | 10 |
| Vitamin B₁ | mg | 0-50 | 0.5-10 | 2.5 | 2.5 |
| Vitamin B₂ | mg | 0-50 | 0.5-25 | 2.0 | 2.0 |
| Vitamin B₃ | mg | 0-60 | 5-40 | 20 | 20 |
| Vitamin B₆ | mg | 0-50 | 0.4-30 | 2.5 | 2.5 |
| Vitamin B₉ | mg | 0-2 | 0.2-1.0 | 0.8 | 0.8 |
| Vitamin B₁₂ | μg | 0-1,000 | 2.0-18 | 8 | 8 |
| Biotin | mg | 0-15 | 0.05-15 | 0.3 | 0.3 |
| Mineral | | | | | |
| Pantothenic | mg | 0-20 | 3-15 | 10 | 10 |
| Acid | | | | | |
| Calcium | g | 0-3 | 0.2-2.0 | 1.3 | 1.3 |
| Phosphorus | g | 0-2 | 0.1-1.5 | 1.3 | 1.3 |
| Iodine | μg | 0-200 | 45-150 | 150 | 150 |
| Iron | mg | 0-100 | 5-50 | 18 | 18 |
| Magnesium | mg | 0-600 | 50-500 | 450 | 450 |
| Copper | mg | 0-2 | 0.1-2 | 2 | 2 |
| Zinc | mg | 0-30 | 1-25 | 15 | 15 |
| Selenium | μg | 0-400 | 60-100 | 60 | 70 |
| Manganese | mg | 0-5 | 0.1-5 | 5 | 5 |
| Chromium | μg | 0-150 | 0.1-120 | 25 | 25 |
| Molybdenum | μg | 0-75 | 20-75 | 25 | 25 |
| Chloride | mg | 0-3,400 | 2,000-3,400 | Not Established | Not Established |
| Choline | mg | 0-1,000 | 300-600 | 450 | 550 |
| Fluoride | mg | 0-5 | 1-4 | Not Established | Not Established |
| Potassium | mg | 0-80 | 10-80 | Not Established | Not Established |
| Sodium | mg | 0-2,400 | 10-1,000 | Not Established | Not Established |

One 8-fluid ounce or other serving of the ready-to-drink liquid form of the nutritional supplements of the invention, or one 60-gram or other serving of the food bar or other solid forms of the nutritional supplements of the invention, may contain one or more of the above (or other) vitamins and/or minerals in their preferred or other quantity range (or in their more preferred range, or in the most preferred quantity for pregnant women, lactating women or women having childbearing potential that are attempting to become pregnant, or for other humans), or in at least 100% of the U.S. RDI for the vitamin and/or mineral, unless otherwise stated. Alternatively, one 8-fluid ounce, one 60-gram or some other sized serving of the nutritional supplements may contain each of these vitamins and/or minerals in one half, one third, one forth, one fifth, one sixth, and so forth, of these quantities. Varying combinations of the vitamins and minerals may also be employed.

### Chromium

Chromium (Cr) is a chemical element in the period table, and has been determined to be an essential micronutrient for the maintenance of normal glucose tolerance in animals. Trivalent chromium (chromium (III)) is an essential trace metal, and is required for the proper metabolism of sugar in humans. Chromium deficiencies can affect the potency of insulin in regulating blood sugar balance.

Although many foods naturally contain adequate dietary levels of chromium for humans, the chromium is generally depeleted during food processing. Thus, it has become increasingly important to supplement the human diet with chromium.

The nutritional supplements of the invention may optionally, but preferably, include chromium.

The amount of chromium that may be included in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for maintaining, or aiding in the maintenance of, normal blood glucose levels, or returning, or aiding in the return of, abnormal glucose levels to normal blood glucose levels, in a human that has, is at risk for, or develops borderline diabetes, diabetes, other glucose intolerance or cardiovascular disease. This amount may vary depending upon the type, age, health and status of the humans that are to consume the nutritional supplements, and like considerations, but generally ranges from about 60 mcg to about 500 mcg per day, and more preferably is at least about 100% of the U.S. RDI for chromium.

Preferably, a daily quantity of the nutritional supplements of the invention includes at least about 100% U.S. RDI of chromium, and one 8-fluid ounce serving of a ready-to-drink liquid nutritional supplement of the invention includes chromium in an amount ranging from about 0.05 mg to about 0.20 mg, with about 0.12 mg being more preferred.

Because Chromium (VI) is an established human carcinogen that can be toxic if orally ingested, chromium (VI) should not be included in the nutritional supplements of the invention.

### Selenium

Selenium (Se) is a mineral that is required by human beings, and that occurs in several different forms, including sodium selenate and sodium selenite. Selenium has antioxidant properties, and is associated with fat metabolism.

Selenium may, optionally, be included in nutritional supplements of the invention, and is preferably included in the form of sodium selenite, rather than sodium selenate.

Selenium may be included as an ingredient in nutritional supplements of the invention in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for enhancing the metabolism of fat in a human and/or acting as an antioxidant. This amount may vary depending upon the type, age, health and status of the humans that are to consume the nutritional supplements, and like considerations, but generally ranges from about 10 mcg to about 200 mcg per day, and preferably ranges from about 20 mcg to about 70 mcg per day.

### L-Arginine

Arginine is one of the 20 most common natural amino acids, and is often classified as one of the 10 “essential amino acids” for children. L-arginine has been shown to significantly improve peripheral and hepatic insulin sensitivity in type-2 diabetic patients. Some studies have also shown improved recovery of T-cell function (immunity) with the administration of L-arginine. L-arginine also appears to act synergistically with DHA.

L-arginine is difficult to obtain from foods consistently at levels that can assist with circulatory disorders in borderline diabetic, diabetic, geriatric and other humans, and in humans that have, are at risk for, or develop glucose intolerance or cardiovascular disease.

The nutritional supplements of the invention contain L-arginine in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for enhancing vascular, neurological and/or immune function in humans and/or peripheral and/or hepatic insulin sensitivity in diabetic patients. This amount may vary depending upon the type, age and health of the humans that are to consume the nutritional supplements, and like considerations, but generally ranges from about 20 mg to about 3 g per day, and preferably ranges from about 500 mg to about 1.5 g per day, with about 1 g being most preferred.

Preferably, one 8-fluid ounce serving of a ready-to-drink liquid nutritional supplement of the invention includes about 1 g of L-arginine.

### L-Carnitine

Carnitine is an amino acid that is responsible for a transport of fatty acids into the mitochondria of cells, and enhances a metabolism of fats and/or oils, which is beneficial for obese humans, who are at risk for diabetes or other glucose intolerance. As with all amino acids that are used directly in the metabolism, carnitine exists in the left-handed form (as the isomer L-carnitine).

The nutritional supplements of the invention contain L-carnitine in an amount that preferably is safe for consumption by humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for enhancing a transport of one or more fatty acids into mitochondria present in cells of the human, a transformation of one or more edible fats and/or oils that are present in the nutritional supplements into energy (a metabolism of the fats and/or oils) and/or cardio-pulmonary function in the human. This amount may vary depending upon the type, age, health and status of the humans that are to consume the nutritional supplements, and like considerations, but generally ranges from about 10 mg to about 150 mg per day, and preferably ranges from about 30 mg to about 90 mg per day.

Preferably, one 8-fluid ounce serving of a ready-to-drink liquid nutritional supplement of the invention contains about 31 mg of L-carnitine.

### Taurine

Taurine (C₂H₇NO₃S), which is also known as 2-aminoethanesulfonic acid, is not an essential nutrient in humans. Although taurine contains an amino group, it lacks the requisite carboxyl group of amino acids and, thus, is not an amino acid. There is some evidence that taurine is an inhibitory neurotransmitter in the central nervous system, and plays a role in digetion.

Taurine may, optionally, be included in the nutritional supplements of the invention. Thus, nutritional supplements of the invention may, or may not, include detectable levels of taurine (as measured using conventional detection processes and equipment), or any taurine. Methods for detecting taurine include known high performance liquid chromatographic (HPLC) methods.

If taurine is included in nutritional supplements of the invention, the nutritional supplements should contain an amount of taurine that preferably is safe for consumption by all humans, including diabetic, borderline diabetic and geriatric humans, pregnant or lactating women, women having childbearing potential that are attemptiong to become pregnant, and humans that have, are at risk for, or develop other glucose intolerance or cardiovascular disease, and is safe for the developing fetuses and/or breast-fed babies of pregnant or lactating women, and that is effective for maintaining or enhancing the health of the central nervous system or enhancing digetion of foods, or components thereof. This amount may vary depending upon the type, age, health and status of the humans that are to consume the nutritional supplements, and like considerations, but generally ranges from about 10 mg to about 200 mg per day, and preferably ranges from about 25 mg to about 50 mg per day.

Preferably, one 8-fluid ounce serving of a ready-to-drink liquid nutritional supplement of the invention contains about 50 mg of taurine.

### M-Inositol

Inositol (C₆O₆H₁₂) plays an important role as a second messenger in a cell. It is a low-weight diffusible molecule that is used in signal transduction to relay a signal within a cell, and is involved in cytoskeleton assembly, nerve guidance, controlling intracellular calcium (Ca²⁺) concentrations and maintaining membrane potential of the cell.

The nutritional supplements of the invention may, optionally, include m-inositol. The amount of m-inositol that may be included in the nutritional supplements of the invention preferably is an amount that is safe for consumption by humans, including diabetic, borderline diabetic and geriatric humans, pregnant or lactating women, women having childbearing potential that are attemptiong to become pregnant, and humans that have, are at risk for, or develop other glucose intolerance or cardiovascular disease, and is safe for the developing fetuses and/or breast-fed babies of pregnant or lactating women, and that is effective for enhancing signal transduction within cells, cytoskeleton assembly and/or nerve guidance, and/or for aiding in the control of intracellular calcium concentrations and/or the maintenance of cell membrane potential. This amount may vary depending upon the type, age, health and status of the humans that are to consume the nutritional supplements, and like considerations, but generally ranges from about 20 mg to about 400 mg per day, and preferably ranges from about 100 mg to about 200 mg per day.

Preferably, one 8-fluid ounce serving of a ready-to-drink liquid nutritional supplement of the invention contains about 186 mg of m-inositol.

### Mouthfeel Agents

Nutritional supplements of the invention in the form of ready-to-drink liquids contain one or more mouthfeel agents that are edible, and that have an effect of stabilizing and/or providing mouthfeel and/or texture to the nutritional supplements. These mouthfeel agents may, optionally, be included in nutritional supplements of the invention in the form of food bars or the like.

Mouthfeel agents that may be employed in the nutritional supplements of the invention include, for example, cellulose gum, cellulose gel, gum arabic, carrageenan and gellan gum, and various combinations thereof. Preferably, the nutritional supplements include a mouthfeel “system” that comprises a mixture of gum arabic and carrageenan, for example, a blend of: (a) Avicel® CL-611 (Cellulose Gum/Gel), which is microcrystalline cellulose and carboxymethylcellulose sodium; and (b) Seakem CM614 (Carrageenan), both of which are commercially available from FMC. Inc. Such a mouthfeel system, along with the emulsifier lecithin, provides nutritional supplements of the invention with an emulsification matrix that has a good mouthfeel, which is important for consumer acceptance, and that is safe for consumption by humans.

Gum arabic (acacia gum) can provide stabilization for emulsions, suspensions and general thickening properties, and is naturally derived from plant sources. It can be prepared from an exudate from the stems and branches of sub-Saharan (Sahel zone) Acacia senegal and Acacia seyal (Leguminosae) trees, and produced naturally as large nodules during gummosis processes. Gum arabic is a complex and variable mixture of arabinogalactan oligosaccharides, polysaccharides and glycoproteins.

Carrageenan is a collective term for polysaccharides prepared by alkaline extraction (and modification) from red seaweed (Rhodophycae), mostly of genus Chondrus, Eucheuma, Gigartina and Iridaea. Different seaweeds produce different carrageenans, such as kappa-carrageenan and iota-carrageenan. Carrageenans are linear polymers of about 25,000 galactose derivatives with regular, but imprecise, structures, dependent on the source and extraction conditions. Carrageenans consist of alternating 3-linked-β-D-galactopyranose and 4-linked-α-D-galactopyranose units.

The one or more mouthfeel agents that may, optionally, be included as ingredients in nutritional supplements of the invention may be present in a combined amount that preferably is safe for consumption by all humans, including diabetic, borderline diabetic and geriatric humans, pregnant or lactating women, women that have childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for providing, or enhancing, a stabilization of the nutritional supplements during thermal or other processes (or otherwise), providing a desirable mouthfeel and/or texture to the nutritional supplements, enhancing a mouthfeel or texture of the nutritional supplements and/or causing the nutritional supplements to become more thick. This amount may vary depending upon the mouthfeel and texture desired, and upon the type, age, health and/or status of the humans that are to consume the nutritional supplements, and like considerations, but generally ranges from a combined amount of about 0.2 to about 2.0 weight percent of the total weight of the nutritional supplements, and preferably ranges from about 0.5 to about 1.0 weight percent.

### Gellan Gum

Gellan gum is a high molecular weight extracellular heteropolysaccharide produced by fermentation of the organism Sphingomonas elodea (previously called Pseudomonas elodea), and may be employed as a mouthfeel agent. When fermentation of the organization is complete, the viscous broth can be pasteurized to kill viable cells prior to recovery of the gum. Direct recovery from the broth yields the gum in its native or high acyl form. Recovery after deacylation by treatment with alkali produces the gum in its low acyl form.

Gellan gum may, optionally, be included within the nutritional supplements of the invention. Thus, nutritional supplements of the invention may, or may not, include detectable levels of gellan gum (as measured using conventional detection processes and equipment), or any gellan gum. Methods for detecting gellan gum include known high performance liquid chromatographic (HPLC) methods.

### Lutein

Lutein is an antioxidant that cannot be manufactured by the body, and that supports eye and skin health, as well as cardiovascular health, when consumed. It is present in vegetables (spinach, kale, broccoli and corn), fruit (oranges and tangerines) and egg yolks, and is commercially available from known sources, such as American Nutrition (Castle Rock, Colo.). In particular, lutein aids in the prevention of retinal degeneration.

The nutritional supplements of the invention include lutein as an ingredient therein. Lutein may be included in the nutritional supplements of the invention in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for maintaining, or enhancing, eye, skin and/or cardiovascular health. This amount may vary depending upon the type, age, health and status of the humans that are to consume the nutritional supplements, and like considerations, but generally ranges from about 0.2 mg to about 2.0 mg per day, and preferably ranges from about 0.5 mg to about 1.0 mg per day.

Preferably, one 8-fluid ounce serving of a ready-to-drink liquid nutritional supplement of the invention contains about 0.5 mg of lutein.

### Antioxidant Agents

Oxidation is a process that can cause an edible fat or oil to become “rancid,” and food products including such edible fat or oil to lose their “freshness,” become less stable and/or have a reduced shelf life.

Edible fats and oils, whether or not they are present in a food product, are generally susceptible to “oxidative rancidity.” The edible fats and oils are susceptible to attack by oxygen, which is present in air, and generally causes the edible fats and oils to eventually become “rancid” and, consequently, to have or develop a “rancid” taste and odor. The oxygen causes the edible fats and oils to oxidize, degrade and/or otherwise decompose (“oxidize”), and to form oxidative products, such as aldehydes, particularly when the edible fats and oils, or food products in or on which they are present, are maintained in an environment that is not cool.

Antioxidant agents are agents having an ability to prevent, reduce or aid in the prevention or reduction of, an oxidation, degradation and/or other decomposition (hereinafter “oxidation”) of one or more food product ingredients, such as an edible fat or oil, and/or to prevent, or aid in the prevention of, oxygen-based damage to cells.

Edible antioxidant agents include, for example, ascorbyl palmitate, various tocopherol mixtures, edible acids (citric acid, ascorbic acid and the like), vitamin A, vitamin C, vitamin E, beta-carotene, selenium, magnesium, herbal extracts, such as a Rosemary, Sage, Oregano, Ginger, Marjoram or Rosemary Oleoresins extract, plant phenols, such as Vanillin, ellagic acid and Resveratrol, and synthetic antioxidants, such as tertiary butylhydroquinone (TBHQ), butylated hydroxyamisole (BHA) or butylated hydroxytoluene (BHT), or mixtures thereof. Antioxidant agents that are particularly beneficial in human nutrition include vitamin A, vitamin C, vitamin E, beta-carotene, ascorbyl palmitate and selenium.

In addition to lutein, the nutritional supplements of the invention include one or more other antioxidant agents.

In the nutritional supplements of the invention, the salts and forms of nutrients are preferably carefully balanced in a unique matrix to permit one or more antioxidant agents to supply critical nutritional needs for humans while, at the same time, preventing an oxidation of one or more fats and/or oils included within the nutritional supplements, particularly the relatively fragile DHA molecule. Additionally, the quantities of powerful prooxidants, such as iron and copper that may be included in the nutritional supplements are carefully balanced so as to be at a useful level to the human body, but at a level that interferes little with oxidative prone nutrients in the matrix of the nutritional supplements. The matrix is preferably weighted to the antioxidant components and content (vitamin A, beta-carotene, vitamin E, vitamin C, selenium and the like), and slighted towards the prooxidants iron and copper. This provides the human body with an abundance of nutritionally desirable antioxidant agents in a matrix, wherein the antioxidant agents also preserve the “freshness” of the nutritional supplements by reducing an oxidative effect upon these supplements.

The one or more antioxidant agents that are included in the nutritional supplements of the invention (in addition to lutein) may be employed in a combined amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, and/or enhancing a stability and/or shelf life of the nutritional supplements, or of one or more ingredients included therein. This quantity will vary depending upon the one or more antioxidant agents that are chosen for use, and upon the type, age, health and status of the humans that are to consume the nutritional supplements, and like considerations, but generally ranges from a combined amount of about 0.01 to about 3.0 weight percent of the total weight of the nutritional supplements, with about 0.1 weight percent being preferred.

### Emulsifiers

The nutritional supplements of the invention typically include both water-soluble ingredients and fat-soluble ingredients, as well as oils and/or fats and water. In order to combine such ingredients, and to maintain them in a stable formula, nutritional supplements of the invention include one or more edible emulsifiers. Soy lecithin is a preferred emulsifier for inclusion in the nutritional supplements.

The one or more emulsifiers that are included in the nutritional supplements of the invention may be employed in an amount (combined) that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for forming, or aiding in the formation of, an emulsion, suspending solids within the nutritional supplements and/or emulsifying lipids within the nutritional supplements. This quantity will vary depending upon the particular emulsifiers that are chosen for use, and upon the type, age, health and status of the humans that are to consume the nutritional supplements, and like considerations, but generally ranges from a combined amount of about 0.2 to about 2.0 weight percent of the total weight of the nutritional supplements, and preferably range from about 0.5 to about 1.0 weight percent.

### Flavoring, Sweetening and Taste-Masking Agents

The nutritional supplements of the invention may, optionally, contain one or more of a wide variety of edible natural or artificial flavoring agents (to produce a wide variety of different flavors of the drinks), sweetening agents and/or taste-masking agents which, alone or in a plurality, may provide sweetness or one or more of a wide variety of food flavors to the nutritional supplements, and/or may mask (reduce or eliminate) an unpleasant taste that an ingredient of the nutritional supplements, such as an edible fat or oil, may have or otherwise impart to food components or products. The nutritional supplements of the invention may have any flavor that is known and/or that may be used in food ingredient or food products, and are preferably naturally and/or artificially flavored in a manner that encourages consumer compliance.

The nutritional supplements of the invention generally have a pleasant taste (which is often excellent), whether or not one or more flavoring agents and/or taste masking agents are included in the nutritional supplements, and whether or not the edible fats and/or oils present therein are hydrogenated. Further, these nutritional supplements generally also have a pleasant smell.

As a result, in part, of one or more taste-masking agent(s) and/or flavoring agents that may, optionally, be chosen for use, nutritional supplements of the invention may be flavored as, taste and/or smell like cocoa, chocolate, chocolate fudge, peanut butter, butter pecan, vanilla, French vanilla, mocha, coffee, maple, caramel, coconut, brown sugar, white sugar, honey, molasses, corn syrup, marshmallow, citrus oils (lemon oil, oil of orange juice, etc.), various fruit flavors (lemon, lime, orange, grape, pineapple, grapefruit, banana, cherry, raspberry, strawberry, pineapple, kiwi, apple, peach, plum, pear, etc.) or a combination of the foregoing flavors. Alternatively, the nutritional supplements may have some other taste or food flavor. Those of ordinary skill in the art may readily determine the taste and smell that various nutritional supplements of the invention may have depending upon the factors described above.

Flavoring and taste-masking agents for edible fats and/or oils, and other ingredients that may be employed in the nutritional supplements of the invention, are commercially available from sources known by those of skill in the art, and include, but are not limited to, high fructose corn syrup, corn syrup, oligofructose, honey, cocoa powder, coffee, sugar (white, brown, powdered, etc.), chocolate, unsweetened chocolate, maltodextrins, natural flavors, for example, citrus, acidic and other natural flavors (grapefruit, grapefruit rind, lemon, lemon rind, lime, lime rind, orange, orange rind, tangerine, cherry, raspberry, blueberry, banana, mango, grape, apple, pear, peach, plum, watermelon, pineapple, coconut, kiwi, etc., or the juice from any of the foregoing) and other components that are known to flavor food products, or to mask the taste of unpleasant tasting food product ingredients. When taste-masking agents are employed in nutritional supplements of the invention, natural flavors and maltodextrins are preferred for use as taste-masking agents.

Artificial sweeteners, such as aspartame, acesulfame, saccharin, cyclamate and Sucralose may be employed to flavor and/or sweeten the nutritional supplements of the invention. Saccharin is a sweetener that has no calories and no nutritional value.

Sucralose, which is sold under the brand name Splenda by McNeil Nutritionals LLC (Washington, Pa.) is a preferred sweetener for use in the nutritional supplements of the invention. Sucralose is non-caloric sweetener that is about 600 times sweeter than the sugar sucrose. It is stable under heating so that, unlike aspartame, it can be used in baking. Sucralose may assist with nutritional requirements of a borderline diabetic, diabetic or other mammal, and in satisfying their craving for sweet food items and certain traditional foods that are contraindicated in diabetes or cardiovascular conditions.

The one or more flavor agents and/or taste-masking agents that may, optionally, be included as ingredients of nutritional supplements of the invention may be present in an amount (combined) that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for flavoring or sweetening the nutritional supplements, or for enhancing the flavor or sweetness of the nutritional supplements, and/or masking (reducing or eliminating) the taste of one or more unpleasant food product ingredients. This amount may vary widely depending upon the results desired to be achieved, and upon the type, age, health and status of the humans that are to consume the nutritional supplements, and like considerations, but generally ranges from a combined amount of about 0 to about 40 weight percent of the total weight of the nutritional supplements, and preferably ranges from about 20 to about 30 weight percent. The most preferred quantity will vary depending upon the number and type of the flavoring agents and/or taste-masking agents employed in the nutritional supplements, and may readily be determined by those having ordinary skill in the art.

Each of the flavoring agents and/or taste-masking agents employed in the nutritional supplements of the invention may be used in any ratio with other flavoring agents and/or taste-masking agents employed in the nutritional supplements that is effective for providing as least some flavor to the nutritional supplements and/or masking the taste of one or more food product ingredients, that is safe for consumption by humans and that is not harmful to developing fetuses or breast-fed babies of pregnant or lactating women.

It is preferable that an 8-ounce serving of a nutritional supplements of the invention contain a total of about 14 grams or less of one or more flavoring agents, sweetening and/or taste-masking agents, whether the ingredients are used as a primary or secondary sweetening or taste-masking function and includes artifical and natural ingredients. In addition, it is preferable that an 8-ounce serving of the nutritional supplements contain about 5 g of sugars, which is lower in amount than the 7 g contained in Glucerna.

### Bitterness and Off-Taste Masking Agents

One or more agents that partially or completely mask a bitter, strong, unpleasant and/or off taste or odor of one or more ingredients that are included in the nutritional supplements of the invention (generally ingredients other than edible fats and/or oils) may also, optionally, be included in the nutritional supplements. Certain vitamins and/or minerals have a bitter, strong and/or off taste or odor, particularly the B vitamins, which generally have strong aromas and tastes, minerals, which often have unpleasant metallic tastes, and herbs and botanicals, such as Kava Kava and Ginko Biloba, which have strong bitter tastes.

A wide range of bitterness and off-taste masking agents in various forms (liquid, powder or solid) that may be employed in the nutritional supplements of the invention may be obtained from sources known by those of skill in the art, such as Flavor Savor, Inc. (Franklin Park, Ill.). Some of the agents that are available from Flavor Savor, Inc., include No. 598.605/T, No. 597.492/A, No. 598.876/TP05.51, No. 598.118/AP10.51, No. 861.016/TD10.90 and No. 861.472/TD10.90.

The one or more bitterness and/or off-taste masking agents that may, optionally, be employed in the nutritional supplements of the invention may be employed in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for partially or completely masking a bitter, strong, unpleasant and/or off taste or odor of one or more ingredients that are included in the nutritional supplements. This quantity will vary depending upon the particular bitterness and/or off-taste masking agents that are chosen for use, and upon the type, age, health and status of the humans that are to consume the nutritional supplements, and like considerations, but generally ranges from a combined amount of about 0 to about 3 weight percent of the total weight of the nutritional supplements, and preferably ranges from about 0.05 to about 2 weight percent, with about 0.5 weight percent being most preferred.

### Preservatives

One or more preservatives, such as sodium benzolate, ascorbyl palmitate, sorbic acid, BHT or EDTA may, optionally, be employed in the nutritional supplements of the invention, for example, to increase the shelf life of the final products.

The one or more preservatives that may, optionally, be employed in the nutritional supplements of the invention may be employed in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for enhancing a stability and/or shelf life of the nutritional supplements, and/or of one or more of the ingredients included therein, preventing or reducing an oxidation, degradation, decomposition, chelation and/or hydrolysis of one or more ingredients that are included in the nutritional supplements and/or preventing or reducing an ability of the nutritional supplements to lose vitamin and/or mineral potency. This quantity will vary depending upon the particular preservatives that are chosen for use, and upon the type, age, health and status of the humans that are to consume the nutritional supplements, and like considerations, but generally ranges from a combined amount of about 0 to about 5 weight percent of the total weight of the nutritional supplements, and preferably ranges from about 1 to about 3 weight percent, with about 1 weight percent being most preferred.

Sodium benzolate is a preservative that may aid in the prevention of the oxidation, chelation or hydrolysis of one or more ingredients that are included in the nutritional supplements of the invention and, thus, can prevent or reduce an ability of the nutritional supplements to lose vitamin and/or mineral potency, or to develop off flavors.

### Anti-Constipation and Regularity-Maintaining Agents

A wide variety of anti-constipation and regularity-maintaining agents may, optionally, also be employed in the nutritional supplements of the invention in varying quantities.

Anti-constipation and regularity-maintaining agents often increase the bulk or water content of stools, making bowel movements more regular and/or comfortable. The anti-constipation and regularity-maintaining agents may be bulk-forming ingredients (ingredients that increase the bulk volume and water content of the stool, thereby promoting bowel movements) or laxative ingredients.

Some of the known naturally-occurring, chemical, medicinal, prescription or over-the-counter agents for maintaining regularity and/or reducing or eliminating constipation include, for example, inulin (and other fructooligosaccharides), agar, alpha cellulose, linseed (whole, crushed or rough ground), psyllium, psyllium seed, psyllium seed (blond), psyllium seed husk, psyllium hydrophilic mucilloid, calcium lactate coated psyllium, kelp, Plantago ovata husks, polycarbophil, calcium polycarbophil, linolenic acid, carrageenan (degraded or native), guar gum, malt soup extract, karaya, methylcellulose, carboxymethylcellulose, sodium carboxymethylcellulose, docusate sodium, bran (wheat, corn, barley, rye, oats, cereal or other grain products, particularly in whole form), flax, flaxseed (whole, crushed or rough ground), flaxseed oil, beans, nuts, seeds, fruit (particularly when the skin is present), pectin, vegetables (particularly raw, and when the skin is present), diphenylmethane derivatives, such as phenolphthalein and bisacodyl, saline and osmotic laxatives (various Mg²⁺ salts, such as magnesium sulfate, magnesium citrate, and magnesium hydroxide, the sulfate, phosphate and tartrate salts of Na⁺ or K⁺, such as sodium phosphate, sodium biphosphate, sodium sulfate, sodium bicarbonate, sodium chloride, potassium chloride, potassium sodium tartrate, and tartaric acid and its salts), castor oil, mineral oil, the dissacharides lactulose, glycerin and sorbitol, and other forms of soluble and insoluble fiber, all of which are commercially available from sources known in the art, and mixtures thereof. Calcium polycarbophyll, inulin, psyllium, methylcellulose, wheat fiber and bran are preferred anti-constipation and regularity-maintaining agents for use in the nutritional supplements of the invention.

Psyllium Fiber

Psyllium fiber is the coat of the psyllium seed (either intact or comminuted), which come from plants of the Plantago genus. The seed coats can be removed and sanitized by methods known in the art, such as ethylene oxide sanitation or steam sanitation, as described in U.S. Pat. No. 4,911,889.

Inulin

Inulin is a natural food ingredient that is found in more than 36,000 plants including leeks, onions, artichokes, garlic and wheat. It is also present in large amounts in chicory roots and some other herbs, such as burdock root, dandelion root, elecampane root and the Chinese herb codonopsis. Oligofructose is a natural constituent of inulin. Inulin and oligofructose are composed of linear chains of fructose units linked by β(2-1) bonds and often are terminated by a glucose unit. Inulin contains chains with up to 60 fructose units. Oligofructose has between 2 and 7 fructose units.

Inulin is not digested or absorbed. Like some pectins and fructooligosaccharides, inulin is a preferred food for the lactobacilli in the intestine, and can improve the balance of friendly bacteria in the bowel. Bifidobacteria digest inulin to produce short chain fatty acids, such as acetic, propionic, and butyric acids. Recent animal research suggests that inulin prevents precancerous changes in the colon. Mark Izzo et al., “Anatomy of a claim, A Case Study of Calcium Absorption claims on Dairy Products,” which appeared in Dairy Foods Magazine (2003), describes a form of inulin (RAFTILOSE® Synergy 1) that is enriched and formulated to maximize calcium absorption, and states that this form of inulin is clinically proven to increase calcium absorption.

Inulin may be extracted from chicory roots and other sources using hot water. Oligofructose (inulin hydrolysates) may be obtained from inulin using a known partial enzymatic hydrolysis technique. Inulin products and other fructooligosaccharides, such as the RAFTILINE®, RAFTILINE®HP, RAFTILINE®HP-GEL, RAFTILINE®ST, RAFTILINE®GR, RAFTILINE®ST GEL, RAFTILOSE®, RAFTILOSE® Synergy 1, RAFTILOSE®L60, RAFTILOSE®P95 and RAFTIFEED® inulin products, are also commercially available from Orafti Active Food Ingredients (Malvern, Pa.). The components of some of these products are set forth below.

### RAFIILOSE® Synergy 1

| | Average Values Expressed |
|---|---|
| Component | per 100 g of Product |
| Calories | 208 |
| Total fat (g) | 0.0 |
| Saturated fat (g) | 0.0 |
| Cholesterol (mg) | 0.0 |
| Sodium (mg) | 8.0 |
| Total Carbohydrate (g) | 96.5 |
| Dietary fiber (g) | 88.8 |
| Sugars (g) | 7.7 |
| Protein (g) (N × 6.25) | 0.1 |
| Calcium (mg) | 12.0 |
| Iron (mg) | 0.1 |
| Soluble fiber (g) | 86.0 |
| Insoluble fiber (g) | 0.0 |
| Potassium (mg) | 7.0 |
| Moisture | 3.0 |
| Ash (g) | 0.2 |

### RAFTILOSE®P95

| | Average Values Expressed |
|---|---|
| Component | per 100 g of Product |
| Calories | 206 |
| Total fat (g) | 0.0 |
| Saturated fat (g) | 0.0 |
| Cholesterol (mg) | 0.0 |
| Sodium (mg) | 1.5 |
| Total Carbohydrate (g) | 96.5 |
| Dietary fiber (g) | 90.0 |
| Sugars (g) | 6.5 |
| Protein (g) (N × 6.25) | 0.1 |
| Calcium (mg) | 0.5 |
| Iron (mg) | 0.1 |
| Soluble fiber (g) | 90.0 |
| Insoluble fiber (g) | 0.0 |
| Potassium (mg) | 1.0 |
| Moisture | 3.0 |
| Ash (g) | 0.2 |

Additional information concerning inulin is present at the web site www.orafti.com.

Some fiber is soluble in water, and other fiber is insoluble. Some fibers are indigestible, and pass through the body virtually intact. Fibers aid in digestion, add bulk to stools, and hasten the passage of fecal material through the abdomen, thus, helping to prevent or alleviate constipation, and related hemorrhoids. Foods containing high levels of soluble fiber are dried beans, oats, barley and some fruits, such as apples and citrus, and vegetables, such as potatoes. Foods high in insoluble fiber are wheat bran, whole grains, cereals, seeds and the skins of many fruits and vegetables.

Additional information concerning agents used to maintain regularity of bowel movements and/or to reduce or eliminate constipation is present in The Merck Index (Eleventh Edition, Merck & Co., Inc., Rahway, N.J., 1989), in Goodman and Gilman's The Pharmacological Basis of Therapeutics (Eight Edition, Pergamon Press, New York, 1990), in J. O. Greenhalf, “Laxatives in the Treatment of Constipation in Pregnant and Breast-Feeding Mothers,” The Practitioner, 210, 259-263 (1973), and in 21 CFR Part 334 (“Laxative Drug Products for Over-the-Counter Human Use”).

The one or more anti-constipation and regularity-maintaining agents that may, optionally, be employed in the nutritional supplements of the invention may be employed in an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for maintaining regular bowel movements and/or reducing or eliminating constipation. This quantity will vary depending upon the particular anti-constipation and regularity-maintaining agent(s) used, and upon the type, age, health and status of the humans that are to consume the nutritional supplements, and like considerations, but generally ranges from a combined amount of about 0.5 to about 5.0 weight percent of the total weight of the nutritional supplements, and preferably ranges from about 0.5 to about 3.0 weight percent.

It is preferable that one 8-fluid ounce serving of a ready-to-drink liquid nutritional supplement of the invention include about 1.20 weight percent fiber of the total weight of the nutritional supplement.

A typical dose of psyllium for Taxation purposes involves the administration of from about 3 to about 15 grams of psyllium fiber in one dose. Calcium polycarbophyll is generally administered to pregnant women, lactating women or women having childbearing potential that are attempting to become pregnant in a daily amount ranging from about 1 to about 5,000 mg, preferably ranging from about 1,250 to about 5,000 mg, with about 1,250 mg being most preferred (for each category of women). Calcium polycarbophyll may also be administered to other humans in such amounts.

Flax is generally administered to pregnant women, lactating women or women having childbearing potential that are attempting to become pregnant in a daily amount ranging from about 1 to about 5,000 mg, preferably ranging from about 1,000 to about 3,000 mg, with about 1,000 mg being most preferred (for each category of women). Linolenic acid is generally administered to pregnant women, lactating women or women having childbearing potential that are attempting to become pregnant in a daily amount ranging from about 1 to about 300 mg, preferably ranging from about 10 to about 200 mg, with about 100 mg being most preferred (for each category of women). Flax and linolenic acid may also be administered to other humans in such amounts.

The chart below shows the amount of RAFTILOSE®P95, RAFTILINE®HP, RAFTILINE®HP-GEL, RAFTILINE®ST, RAFTILINE®GR, RAFTILINE®ST GEL and RAFTILOSE® Synergy 1 inulin products that should be consumed to obtain 1 g of fiber, a good source of fiber (2.5-4.9 g per serving) or high fiber (5 g per serving).

| | | | g Needed to | g Needed to |
|---|---|---|---|---|
| | Minimum | g Needed to | Obtain Good | Obtain High |
| Product | Fiber Content | Obtain 1 g Fiber | Source of Fiber | Source of Fiber |
| RAFTILOSE | 89% | 1.12 g | 2.8 g | 5.6 g |
| ® P95 | | | | |
| RAFTILINE | 95% | 1.05 g | 2.6 g | 5.3 g |
| ® HP and HP-GEL | | | | |
| RAFTILINE ® ST, | 86% | 1.16 g | 2.9 g | 5.8 g |
| GR and ST GEL | | | | |
| RAFTILOSE ® | 86% | 1.16 g | 2.9 g | 5.8 g |
| Synergy 1 | | | | |

When an anti-constipation and regularity-maintaining agent is employed in nutritional supplements of the invention, and particularly when such agent is wheat bran or another type of a fiber, it is preferable that the agent be reduced to a relatively small particle size, prior to being incorporated into the nutritional supplements. Such particle sizes can be achieved using methods and equipment known by those of skill in the art, such as milling or micronizing techniques. When anti-constipation and/or regularity-maintaining agents are incorporated into nutritional supplements in this manner, the anti-constipation and regularity-maintaining agents generally have an enhanced bioavailability in the body of the human that consumes the nutritional supplements (i.e., the anti-constipation and/or regularity-maintaining agents have an ability to enter the bloodstream of the mammal more rapidly). Additionally, they may be more palatable when present in the nutritional supplements.

Constipation may be a sign of a serious condition that requires the diagnosis and treatment by a physician. Constipation lasting longer than one week could signify a more serious condition, such as diverticular disease of the colon, irritable bowel, or cancer of the colon. In such cases, the individuals suffering from constipation should visit a physician at the earliest possible time so that the condition can be diagnosed and properly treated.

### Eicosapentaenoic Acid (EPA)

EPA is a fatty acid that has anti-clotting properties, and that has an effect of thinning the blood of humans (by inhibiting clotting) when consumed. As a result of these properties, EPA is gaining recognition for supplementation in diets of humans that have cardiovascular disease. However, as a result of synergistic blood thinning effects of EPA combined with aspirin and other anti-clotting agents, drugs or nutrients such as vitamin E, a medical contraindication for EPA can exist with respect to a long-term consumption of EPA or certain medical treatment programs that employ pharmaceutical blood thinning agents, including aspirin and/or high dose vitamin E supplementation. Blood that is too clotting-inhibited can lead to capillary and/or vascular bleeding, possibly with an increased danger of cerebral bleeding. The blood thinning effects of EPA can, in some instances, produce health hazards, for example, spontaneous bleeding, for certain groups of humans, such as pregnant women.

High levels of EPA that have been present in fish oil along with DHA have been linked to internal bleeding, especially in cranial and during pregnancy.

For the above reasons, when the nutritional supplements of the invention are to be consumed by, or administered to, humans that may experience one or more adverse health effects from EPA, such as pregnant women, or humans that are under a medical treatment program that employs one or more pharmaceutical blood thinning and/or anti-clotting agents, the nutritional supplements of the invention preferably do not contain an amount of EPA that is effective for causing the blood of the human to become thinner and/or to have enhanced anti-clotting properties, and more preferably do not contain a detectable amount of EPA, and most preferably do not contain any EPA. Methods for detecting EPA include known high performance liquid chromatographic (HPLC) methods.

### Omega-3 Fatty Acids

Public health recommendations presently exist for increasing an intake of omega-3 fatty acids, particularly omega-3 VLPUFAs (very long chain polyunsaturated fatty acids, such as DHA) for heart health benefits that can be important for diabetics, as well as the general population.

In addition to other fats and/or oils that are included in the nutritional supplements of the invention, the nutritional supplements contain one or more omega-3 fatty acids, for example, DHA, docosapentaenoic acid, ALA, EPA, eicosatetraenoic acid, moroctic acid, heneicosapentenoic acid, linoleic acid, alpha-linolenic acid, or any combination thereof.

Borderline diabetic and diabetic patients are known to have a significantly greater risk of cardiovascular and retinal degenerative disease, and associated diseases, such as renal or immune dysfunction.

DHA can provide strong anti-inflammatory and cardio protective benefits to humans. The cardiovascular, anti-inflammatory and other dietary benefits of DHA are well documented. DHA is a major component of the brain and retina, and has been shown to reduce cholesterol and increase HDL. It is associated with improved or optimal performance of the immune system, as well as the mitigation of cardiovascular disease, especially in contravening pathologic fribulation.

The primary source of “preformed” DHA (DHA that is available in a final form, rather than in a precursor form) today is fish. Various public health authorities recommend the limited consumption of certain amounts of fish to obtain a meaningful intake of preformed DHA with respect to dietary interventions for cardiovascular and other healthful nutritional support. These expert recommendations include cautionary limits to the amount of fish which should be consumed due to contaminant issues, particularly contamination by heavy metals and industrial or hazardous chemicals. These contaminants are innate to fish and other aquatic animals, and are of particular concern in pregnancy, lactation and long-term exposure because they are difficult to excrete by the body, and can build up in the body over time to toxic levels.

Currently, recommendations for fish oil supplements are generally reserved. The health concerns that are related to a consumption of fish and other aquatic animals are exacerbated with fish oil supplements because of a reduced regulatory oversight of supplements as opposed to foods. Further, even if a reliable high quality fish oil supplement containing only low levels of innate contaminants was available, a further concern with such supplement would be the high concentration of EPA that is generally present with DHA in fish oil.

There is a more limited dietary source of DHA, which is from whole egg consumption. However, a large amount of whole eggs would need to be consumed for a DHA intake to be present at a level that is effective for producing a beneficial health effect. Disadvantageously, a dietary-contravening prohibitive cholesterol component would be attendant with such a high level of egg consumption. Additionally, the extraction and concentration of DHA from eggs is an expensive process, and also has contaminant concerns. Most notably, an undesirable concomitant content of egg phospholipids may be present with oils extracted from eggs, which may cause an undesirable allergic reaction thereto.

It is preferable that DHA be present in the nutritional supplements of the invention (in addition to other fats and/or oils that are included in the nutritional supplements). Such DHA may be obtained from any source, including aquatic animals, for example, fish oil.

For safety reasons, it is preferable that the source of the DHA (and any other omega-3 fatty acids), such as fish oil, and the DHA (and any other omega-3 fatty acids), do not contain a detectable hazardous level of mercury, a detectable hazardous level of any heavy metals, a detectable hazardous level of any pesticides or a detectable hazardous level of any industrial chemicals, as measured using conventional detection techniques (such as those described herein). It is also preferable that the source of DHA (and any other omega-3 fatty acids), such as fish oil, contains at least about 30 weight percent of DHA (or of another omega-3 fatty acid). Such DHA (and other omega-3 fatty acids) can be produced by those of skill in the art using conventional oil or omega-3 fatty acid refinement processes and equipment.

For the same reasons, it is more preferable that any DHA (or other omega-3 fatty acids) that is employed in the nutritional supplements be a high grade DHA (or other omega-3 fatty acid) that is obtained from a source that is not an aquatic animal, for example, DHA (or another omega-3 fatty acid) that is obtained from a plant or fungal source, such as algae, microalgae or other seaweed. Such DHA and other omega-3 fatty acids are generally safe for developing fetuses of pregnant women, as well as for breast-fed babies of lactating women (and for other humans). Most preferably, DHA is obtained from microalgae. Such DHA is commercially available, for example, from Martek Corp. (Columbia, Md.), and should be safer for consumption by pregnant and/or lactating women, or women having childbearing potential that are attempting to become pregnant, who may also have, develop, or be at risk for diabetes, borderline diabetes, other glucose intolerance or cardiovascular disease. Not employing DHA (or other omega-3 fatty acids) that is obtained from fish oil, or other aquatic animals, renders fish oil contaminate restrictions and precautionary issues inapplicable to the nutritional supplements of the invention. Further, and importantly, the above-described contaminant and contravening excessive bleeding problems that may be attendant with an EPA component that is present in fish oil are generally overcome with the use of a plant source of high grade DHA (or other omega-3 fatty acid).

The one or more omega-3 fatty acids, such as DHA, that are present in the nutritional supplements of the invention are present in a combined amount that preferably is safe for consumption by all humans, including diabetic, borderline diabetic or geriatric humans, humans that have, are at risk for, or develop other glucose intolerance or cardiovascular disease, pregnant or lactating women and women having childbearing potential that are attempting to become pregnant, and is safe for the developing fetuses and/or breast-fed babies of pregnant or lactating women, and that is effective for providing, maintaining or enhancing the health of a human's eyes, cardiovascular system, renal system, immunological system and/or neurological system, and/or for providing one or more other nutritional, medical and/or health benefits. This amount may vary depending upon the type, age, health and status of the humans that are to consume the nutritional supplements, and like considerations, but generally ranges from about 20 mg to about 800 mg per day, and preferably ranges from about 20 mg to about 200 mg per day.

Preferably, one 8-fluid ounce serving of a nutritional supplement of the invention contains from about 100 mg to about 200 mg of DHA.

### Edible Fats and/or Oils

In addition to DHA, one or more edible fats and/or oils are included in the nutritional supplements of the invention.

Preferably, the one or more edible fats and/or oils that are included in the nutritional supplements of the invention contain one or more beneficial nutrients, such as other omega-3 fatty acids, omega-6 fatty acids, omega-9 fatty acids and/or essential fatty acids.

Polyunsaturated fats and/or oils generally have anti-inflammatory effects and improve circulation in diabetic, borderline diabetic and geriatric humans, as well as in humans that have, are at risk for, or develop other glucose intolerance or cardiovascular disease, and in other humans.

The American Dietetic Association (ADA) suggests that fat intake should be individualized in diabetics, but that fat intake should generally follow expert recommendations (i.e., American Heart Association, AHA).

The nutritional supplements of the invention preferably are low in fat, in saturated fat, in trans fatty acids and in cholesterol, and are in the form of a blend of two or more edible fats and/or oils (preferably one or both of which contains one or more omega-3 fatty acids).

The nutritional supplements of the invention preferably contain an amount of trans fat that provides about 10% or less of daily energy requirements, an amount of saturated fat that provides about 10% or less of daily energy requirements (and more preferably about 7% or less), an amount of monounsaturated fats that provides from about 1% to about 20% of daily energy requirements, and an amount of polyunsaturated fats that provides from about 1% to about 10% of daily energy requirements.

The nutritional supplements of the invention preferably employ a unique monounsaturated dominant fat blend of high oleic sunflower or safflower oil and high oleic canola oil, along with soy lecithin and the very long chain polyunsaturated fatty acid, DHA. This fat blend preferably has zero trans fatty acids, zero cholesterol, very low saturated fat (about 3% of DV) and is consistent with cardio protective fat intake recommendations of the American Heart Association and general provisions of the American Dietetic Association (in combination with the energy provided by carbohydrates), including those that are also related to an improvement in glucose tolerance.

Nutritional supplements within the invention may or may not include detectable levels of soy oil or high oleic safflower oil (as measured using conventional detection processes and equipment). Methods for detecting soy oil or high oleic safflower oil include known high performance liquid chromatographic (HPLC) methods.

The combined amount of one or more edible fats and/or oils that may be present in the nutritional supplements of the invention is an amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for the developing fetuses and/or breast-fed babies of pregnant or lactating women, and that is effective for providing a human with from about 10% to about 50% of the human's daily energy requirements, and more preferably for providing the human with from about 20% to about 35% of the human's daily energy requirements. Such an amount will vary widely depending upon the numbers, and types, of oils and/or fats that are employed in the nutritional supplements, and like considerations, and may readily be determined by those of skill in the art. Such an amount should be sufficient to help support the human body's nutritional needs for fat, while minimizing a risk for chronic disease, and generally ranges from about 5 g to about 65 g in the nutritional supplements, and preferably ranges from about 5 g to about 30 g, and still more preferably ranges from about 5 g to about 10 g, with about 8 grams being most preferred.

The above amount of oils and/or fats may also provide one or more beneficial anti-inflammatory effects to a human, such as reducing or preventing inflammation, improve a blood circulation in diabetic, borderline diabetic, geriatric or other humans, and/or in humans that have, or at risk for, or develop cardiovascular disease, and/or provide one or more other beneficial nutritional, medical and/or health benefits to humans.

The nutritional supplements of the invention provide a convenient vehicle for an oral consumption of one or more edible fats and/or oils to obtain desirable nutritional, health and/or medical benefits that are known to be provided by such fats and oils.

Any one or more of a wide variety of edible fats and/or oils may be employed in the nutritional supplements of the invention, including, but not limited to, those specifically discussed herein. For the reasons described herein, it is preferred that these fats and/or oils are not obtained from aquatic animals (as described herein), Because of their nutrient and/or medical benefits, edible fats and oils that are preferred for use in the nutritional supplements of the invention are those that are, or are rich in (contain large quantities of), essential fatty acids, omega-3 fatty acids, omega-6 fatty acids and/or omega-9 fatty acids, particularly ARA, AA or GLA.

The edible fats and oils that may be employed in the nutritional supplements of the invention are commercially available from sources known by those of skill in the art. Sources for these edible fats and oils include, for example, Martek Corp. (Columbia, Md.), Ocean Nutrition Canada, Ltd. (Bedford, Nova Scotia, Canada), DSM (Heerlen, NL), Pronova Biocare AS (Lysakar, Norway), Napro AS (Brattvaag, Norway), Berg Lipotech AS (Norway), ADM Food Oils (Decatur, Ill.), Oilseeds International, Ltd. (San Francisco, Calif.) and The Edible Oils Co. (www.edible oils.net).

Edible fats and oils, such as omega-3 oils, are generally commercially available in the ethyl esters, triglycerides or free fatty acids forms.

The ethyl esters form of edible fats and oils result from breaking apart the original triglyceride molecules through the known process of “ethylation,” which is performed to increase or concentrate the level of omega-3 fatty acids, while removing the less desirable fatty acids. Ethyl esters of DHA are available from DSM (Heerlen, NL) under the name Ropufa® 75 n-3 EE, and from other oil suppliers described herein.

The triglycerides form of edible fats and oils generally consists either of an unconcentrated oil, or of a concentrated oil that has been reconverted (re-esterified) back to the tri-glycerides form. In the processing of edible oils, the objective is generally to remove existing free fatty acids, monoglycerides and diglycerides to form more stable compositions of the fatty acids, which is the triglycerides or ethyl esters forms of the fatty acids.

The fatty acids form of edible fats and oils generally results from saponification and neutralization of the triglycerides, using known techniques, whereby the glycerol backbone of the triglycerides is generally severed, leaving only the free fatty acid form. Such processing techniques preferably result in the edible fats and oils having an acid value below about 3.0 mg KOH/g and unsaponified matter below about 1.5% w/w.

Methods are known by those of skill in the art for extracting oil from seeds, plants and fruits, including the known solvent extraction and mechanical extraction (cold processing) methods.

Oils that are extracted from seeds, plants, fruits and other sources may have a pronounced color, flavor and aroma. If the refining of such oils does not cause the amount of nutrients present therein to be reduced or eliminated, it is, thus, preferable that edible fats and oils that are employed in the nutritional supplements of the invention, and that are extracted from other sources, be refined, deodorized and substantially free of flesh, seeds or other solids. Such refinement methods are known by those of skill in the art, and generally result in a clean, “finished” oil product. When some oils, such as Evening Primrose oil, are refined, however, nutrients present in the oils are significantly reduced by the refinement process. Those of skill in the art know which edible fats and oils have their nutrients reduced by refinement processes. Unrefined oils are sometimes referred to as “virgin” or “extra virgin” oils.

Both physical refining and chemical refining can generally be employed to refine fats and oils that can be employed in food products. Physical methods include the known filtering, deodorizing and winterization processes. Chemical methods include the known degumming, neutralization and bleaching processes. Additional information concerning the refinement of oils for use in food products is present at the web site www.sanmarkltd.com/processing.htm.

Oil purification is a process in which crude oils are refined to remove flesh, seeds and other solids, as well as other substances that may contribute to off flavor, off odor, undesirable color or keeping quality. The oils can be purified, or fatty acids can be removed from the oils, by degumming the oils (by washing the oils generally using water, salts and acids in order to remove waxes, phosphates and other impurities therefrom). After the impurities are removed from the oils through degumming, the oils can undergo a neutralization process. Alkali can be mixed with the oils to remove free fatty acids, which can contribute to rancidity. A resulting soap/oil mixture can be heated to, for example, 180° F., and pumped through a separator that removes the soap from the oil.

After the above “chemical” refinements have been made, the oils can be subjected to “physical” refinements. The first of these physical refinements is generally the removal of odor compounds from the oils. This can be accomplished by applying a vacuum steam distillation process to the oils. Steam distillation can be followed by winterization, which involves a cooling of the oils. As a result of this cooling process, some oils become crystallized, and can thereafter be removed from other oils using filtration techniques. This results in the oils being more uniform. After winterization, the oils can be bleached and hydrogenised, which processes can stabilize the oils. Bleaching involves the use of clay to remove color and impurities from the oils. The oils can be bleached by heating, for example, to about 130° F., and mixing them with clay. The mixture can be held for several minutes, and then the hot oil can be filtered from the clay and cooled. Hydrogenation can be completed, following bleaching, by pumping pressurized hydrogen into an agitated tank filled with the oils. This is generally done in the presence of a catalyst metal, such as nickel. Hydrogenation can be performed at a temperature, for example, of about 204° C., and a pressure of, for example, 60 psig.

Similar known methods to those described above can also be employed with edible fats and oils. Further, other methods known by those of skill in the art can also be employed to purify edible fats and oils, or to remove fatty acids from such fats and oils. Methods for processing edible fats and oils are well known in the art. See, for example, Marschner et al. U.S. Pat. No. 4,804,555, entitled “Simultaneous Deodorization and Cholesterol Reduction of Fats and Oils.”

In order to permit edible fats and/or oils employed in the nutritional supplements of the invention to provide maximum health benefits, it is also preferred that the edible fats and oils not be hydrogenated. As discussed hereinabove, the hydrogenation of edible oils by decreasing polyunsaturation, including n-3 fatty acid components, generally correspondingly decreases the health value of such oils.

In the United States, there are currently no Reference Daily Intakes (RDIs), Daily Reference Values (DRVs) or Daily Values (DVs) for essential fatty acids or other edible fats or oils. However, the World Health Organization recommends that pregnant and lactating women consume 5% of their daily caloric intake as essential fatty acids. It is recommended that healthy adults consume 4% to 10% of their daily caloric intake as essential fatty acids.

A preferred fat blend for use in the nutritional supplements of the invention includes:
(a) saturated fat, from any source, in an amount generally ranging from about 5 to about 14 weight percent of the total fat blend, and preferably ranging from about 5 to about 10 weight percent, with about 9 weight percent being most preferred;
(b) monounsaturated fat, from any source, in an amount generally ranging from about 30 to about 80 weight percent of the total fat blend, and preferably ranging from about 50 to about 70 weight percent, with about 69 weight percent being most preferred; and
(c) polyunsaturated fat, from any source, in an amount generally ranging from about 10 to about 40 weight percent of the total fat blend, and preferably ranging from about 15 to about 25 weight percent, with about 22 weight percent being most preferred.

As a result of the nutritional, medical and/or other benefits that they can provide to humans, the preferred edible fats and oils for use in the nutritional supplements of the invention include omega-3 fatty acids, omega-6 fatty acids and omega-9 fatty acids, such as ALA, docosapentaenoic acid, moroctic acid, heneicosapentenoic acid, gamma-linolenic acid, linoleic acid, arachidonic acid and oleic acid. As a result of an enhanced stability, such such fatty acids are preferably present in their ethyl esters or tryglycerides forms.

In order to obtain a good edible fat or oil distribution within a mixture of ingredients used to form nutritional supplements within the invention, edible fats that are solid at room temperature may be heated to a temperature ranging from about 100° F. to about 220° F., and preferably to a temperature ranging from about 160° F. to about 172° F. If a solid fat is not heated enough, it will not melt and, thus, will not mix easily with the other ingredients. However, in order to minimize off-flavors that may result from heating solid fats too long, or at too high of a temperature, or the degrading of vitamins or other ingredients that are mixed with the fats, solid fats should generally only be heated for a period of time, and at a temperature, that is sufficient to transform the solid fats into a liquid or semi-liquid state, and to allow the fats to properly blend with other ingredients.

A wide variety of other (different) edible fats and/or oils may, optionally, be employed to produce nutritional supplements within the invention. Such fats and oils are preferably those that are effective for providing some additional or other nutritional, medical and/or other health benefit to the nutritional supplements, such as increasing the fat content of the nutritional supplements for nutritional purposes, or for otherwise enhancing the nutrition of diabetic, borderline diabetic, geriatric or other humans, including humans that have cardiovascular disease, pregnant women or their developing fetuses, lactating women or their babies, women having childbearing potential that are attempting to become pregnant, or other humans, and that are not harmful to the humans, or to developing fetuses and/or breast-fed babies. These edible fats and/or oils may provide other essential fatty acids that may enhance nutrition, and include, but are not limited to, the edible fats and oils described herein, such as plant oils and plant seed oils (corn oil, soy oil, soybean oil, olive oil, canola oil, cotton seed oil, palm oil, palm kernel oil, coconut oil, sunflower oil, high oleic sunflower oil, safflower oil, high oleic safflower oil, peanut oil, walnut oil, almond oil and the like), lecithin, palmolein oil, mustard seed oil, lard, monoglycerides, diglycerides, butter, margarine, and other animal, vegetable, and marine fats, and milk fats, and the like, which are commercially available from sources known by those of skill in the art, and mixtures thereof. Canola oil, for example, provides alpha-linolenic acid, linoleic acid and oleic acid, and is relatively low in saturated fat. Soybean oil provides an additional source of linoleic acid. Preferred oils for use in this manner are soy oil, high oleic sunflower oil, safflower oil, vegetable oil and/or coconut oil.

The one or more fats and oils (total) that may be employed in nutritional supplements of the invention generally ranges from about 2.0 to about 7.0 weight percent of the total weight of the nutritional supplements, and preferably ranges from about 3.0 to about 5.0 weight percent, with about 4 weight percent being most preferred.

It is preferable that an 8-ounce serving of a nutritional beverage of the invention contain about 8 grams of one or more edible fats and/or oils (combined amount), with about 0.5 g being saturated fats and/or oils, and with no trans fats and/or oils. In contrast, an 8-ounce serving of Glucerna contains 1.0 g saturated fat.

### Soy Polysaccharide

The carbohydrate soy polysaccharide may, optionally, be included within the nutritional supplements of the invention. Thus, nutritional supplements of the invention may, or may not, include detectable levels of soy polysaccharide (as measured using conventional detection processes and equipment), or any soy polysaccharide. Methods for detecting soy polysaccharide include known high performance liquid chromatographic (HPLC) methods.

In contrast with soy polysaccharide, gum arabic, which may also, optionally, be included as a carbohydrate in the nutritional supplements of the invention, does not contain any protein, and is completely non-digestible. Soy polysaccharide contains some protein, which is digestible. Thus, unlike gum arabic, a portion of soy polysaccharide is digestible. As a result of the differences in digestibility between gum arabic and soy polysaccharide, these two substances have different non-absorption characteristics.

### Carbohydrates

Carbohydrates (starches) are one of the three main nutrients in food. Foods that provide carbohydrate are vegetables, fruits, dairy products and sugars. Carbohydrates generally are readily absorbed and utilized, and can provide a source of energy for humans who consume them.

Sugars are a class of carbohydrates that have a sweet taste, and include glucose, fructose and sucrose. These carbohydrates, thus, can also be employed as sweetening agents. See, for example, Carbohydrate Sweeteners in Foods and Nutrition (Academic Press, London, 1980). Humans convert sugars to glucose then ‘glycogen’ (animal sugar).

Sugar alcohols, such as maltitol, sorbitol and isomalt, are not sugars or alcohols, but are carbohydrates. They have nutritional, sweeteneing and diuretic properties.

A wide variety of one or more edible carbohydrates, and combinations thereof, can be employed in the nutritional supplements of the invention. These carbohydrates include any carbohydrates that are customary in the preparation of foods, such as ingestible monosaccharidic or disaccharidic materials, their hydrolysis products, and mixtures thereof, for example, fructooligosaccharides, myoinositol, maltitol, gum arabic, cellulose gel, maltodextrin (preferably having a dextrose equivalece of about 10), hydrolyzed corn starch, dextrose, glucose, sucrose, fructose, lactose, maltose, galactose, sugar alcohols, such as sorbitol, mannitol and xylitol, invert sugar syrups, brown sugar, corn syrup, corn syrup solids, honey, molasses, maple syrup, and the like, which are commercially available from sources known by those of skill in the art, and combinations thereof. Molasses, corn syrup, brown sugar and dextrose are preferred carbohydrates for use in the nutritional supplements of the invention.

Maltitol is a commercially available reduced calorie bulk sweetener that has sugar-like taste and sweetness.

Maltodextrins are the easily digestible carbohydrate products, polymers of dextrose that are obtained from the partial acid or enzymatic hydrolysis of starch, and have a dextrose equivalent of less than 20. Maltodextrins function as bodying agents, bulking agents, texturizers, carriers, diluents, sweeteners and crystallization inhibitors, and are generally sold commercially as dried powders.

The nutritional supplements of the invention preferably include one or more (or all) carbohydrates that are “slow-digesting” carbohydrates (that digest slowly in comparison with glucose), such as fructooligosaccharides, maltodextrin and/or fructose. Glucose and dextrose, in contrast, are “fast-digesting” carbohydrates (digest rapidly). Nutritional supplements of the invention that include “slow-digesting” carbohydrates release glucose into the bloodstream at a slower rate in comparison with conventional nutritional supplements.

The carbohydrates that are present in the nutritional supplements of the invention help render the nutritional supplements effective in maintaining blood glucose levels at, or returning blood glucose levels to, normal blood glucose levels, and maintaining a low glycemic index. Thus, these nutritional supplements are particularly useful for humans that are borderline diabetic, diabetic, geriatric, otherwise have glucose intolerance, are pregnant and/or lactating, or have, are at risk for, or develop cardiovascular disease.

Although glucose may not be included in nutritional supplements within the invention, the nutritional supplements may include digestable sugars that are converted to glucose. The sugars are preferably included at a low level, for example, at a level of 7 grams of a total 29 gram carbohydrate content. Other carbohydrates that may be included in the nutritional supplements of the invention, such as fructooligosaccharides (FOS), are not digestable, or not readily digestable, yet still supply nutritional benefit outside glucose. In this manner, the level of “glucose” can be controlled which, in turn, can help manage or control the glycemic index (blood glucose level) of a human.

The nutritional supplements of the invention also preferably include a blend (a mixture of carbohydrates) of more than one carbohydrate, which permits a human to receive nutrients from different carbohydrates, which carbohydrates preferably are slow-digesting carbohydrates, as well as a unique carbohydrate-fiber complex. The carbohydrates that are included in the nutritional supplements of the invention are preferably included in the form of a carbohydrate blend that generally ranges from about 5 to about 30 weight percent of the total weight of the nutritional supplements, and preferably ranges from about 8 to about 16 weight percent, with about 12 weight percent being most preferred.

A preferred carbohydrate blend for use in the nutritional supplements of the invention includes:
(a) maltodextrin (10DE), in an amount generally ranging from about 40 to about 60 weight percent of the total carbohydrate blend, and preferably ranging from about 45 to about 55 weight percent, with about 49 weight percent being most preferred;
(b) crystalline fructose, in an amount generally ranging from about 15 to about 35 weight percent of the total carbohydrate blend, and preferably ranging from about 18 to about 28 weight percent, with about 23 weight percent being most preferred;
(c) maltitol, in an amount generally ranging from about 5 to about 30 weight percent of the total carbohydrate blend, and preferably ranging from about 10 to about 25 weight percent, with about 20 weight percent being most preferred;
(d) maltodextrin (Fibersol 2), in an amount generally ranging from about 0.25 to about 6 weight percent of the total carbohydrate blend, and preferably ranging from about 1 to about 5 weight percent, with about 3 weight percent being most preferred;
(e) gum arabic, in an amount generally ranging from about 0.25 to about 6 weight percent of the total carbohydrate blend, and preferably ranging from about 0.75 to about 3.0 weight percent, with about 2 weight percent being most preferred; and
(f) cellulose gel, in an amount generally ranging from about 8 to about 35 weight percent of the total carbohydrate blend, and preferably ranging from about 13 to about 26 weight percent, with about 23 weight percent being most preferred.

One or more carbohydrates may be included in the nutritional supplements of the invention in a combined amount that preferably is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating women, and that is effective for providing a human with from about 25% to about 80% of the human's daily energy requirements, and more preferably for providing the human with from about 45% to about 65% of the human's daily energy requirements. Such an amount will vary widely depending upon the numbers, and types, of carbohydrates that are employed in the nutritional supplements, and like considerations, and may readily be determined by those of skill in the art. Such an amount should be sufficient to fulfill the human body's daily nutritional needs for carbohydrate, while minimizing a risk for chronic disease, and generally ranges from about 4 to about 12 weight percent of the nutritional supplements, and preferably ranges from about 6 to about 9 weight percent

One or more sugars may be present in the nutritional supplements of the invention (as a portion of the carbohydrate quantities described hereinabove). The amount of one or more sugars (combined) that is present in the nutritional supplements of the invention preferably does not exceed about 35 weight percent of the total amount of carbohydrates employed in the nutritional supplements, and more preferably does not exceed about 25 weight percent.

It is preferred that one or more sugar alcohols are included in the nutritional supplements of the invention (as a portion of the carbohydrate quantities described hereinabove), but in a limited amount, so as not to cause significant diuretic effects (frequent urination and/or diarrhea) to the human consuming the nutritional supplements. One 8-fluid ounce serving of a ready-to-drink liquid nutritional supplement, or one 60-gram serving of a food bar nutritional supplement, preferably includes from about 3 g to about 15 g of sugar alcohols (out of the total number of grams of carbohydrates), and more preferably includes from about 4 g to about 7 g of sugar alcohols. For example, one 8-fluid ounce serving of a ready-to-drink liquid nutritional supplement that contains 29 g of carbohydrates may, for example, include 6 g (of the 29 g) of sugar alcohols.

The dextrose equivalence of starch that is employed in the nutritional supplements of the invention preferably is about 10.

It is preferable that one 8-ounce serving of a ready-to-serve liquid nutritional supplement of the invention contain about 29 g of carbohydrate, about 5 g of which are sugars, and about 4 g of which are sugar alcohols. In contrast, an 8-ounce serving of Glucerna contains 7 g of sugars and 6 g of sugar alcohols.

### Proteins and Amino Acids

Proteins are complex, high molecular weight organic compounds that consist of amino acids that are joined by peptide bonds. Proteins are one of the three main nutrients in food, and are essential to the structure and function of all living cells. Foods that provide protein include meat, poultry, fish, cheese, milk, dairy products, eggs and dried beans.

In addition to L-arginine and L-carnitine, a wide variety of one or more edible animal or vegetable proteins and/or amino acids are employed in the nutritional supplements of the invention. These proteins and amino acids include, for example, cereal proteins, milk proteins (calcium caseinate, sodium caseinate, sodium calcium caseinate, lactalbumin-casein coprecipitate, purified or refined grades of casein and the like), egg proteins, whey protein, bean proteins, soy proteins (soy protein isolate and the like) and proteins from peanuts, all of which are commercially available from sources known by those of skill in the art, and combinations thereof.

It is preferred that the one or more proteins and/or amino acids that are present in the nutritional supplements of the invention have an ability to supply the human body with essential amino acids in an easily digested form, such as a combination of milk and vegetable proteins. It is also preferred that nutritional supplements of the invention contain a combination or blend of two or more proteins, which combination includes soy protein isolate, such as soy protein isolate, sodium caseinate and sodium calcium caseinate.

The one or more proteins and/or amino acids that are present in the nutritional supplements of the invention preferably are present in an amount (combined) that is safe for consumption by all humans, including diabetic, borderline diabetic and geriatric humans, pregnant or lactating women, women having childbearing potential that are attempting to become pregnant, and humans that have, are at risk for, or develop other glucose intolerance or cardiovascular disease, and is safe for developing fetuses and/or breast-fed babies of pregnant or lactating humans, and that is effective for providing a human with from about 5% to about 50% of the human's daily energy requirements, and more preferably for providing the human with from about 10% to about 35% of the human's daily energy requirements, and most preferably for providing the human with from about 17% to about 20% of the human's daily energy requirements. Such an amount will vary widely depending upon the numbers, and types, of proteins and/or amino acids that are employed in the nutritional supplements, and like considerations, and may readily be determined by those of skill in the art. Such an amount should be sufficient to fulfill the human body's daily nutritional needs for protein, while minimizing a risk for chronic disease, and generally ranges from about 2 to about 10 weight percent of the nutritional supplements, and preferably ranges from about 3 to about 7 weight percent of the nutritional supplements.

It is preferable that an 8-ounce serving of a nutritional beverage of the invention contain a combined amount of about 4.26 weight percent of amino acids and/or protein (in addition to the amounts of L-arginine and L-carnitine described herein).

### Water

The amount of water employed to make the nutritional supplements of the invention will depend, in part, upon the form of the nutritional supplement that is being produced (a ready-to-drink liquid, a food bar or the like), and may vary widely. Generally, the amount of water that is employed for a ready-to-drink liquid form of the nutritional supplements is an amount that is effective for raising the total weight of the nutritional supplement to 100 percent, and to provide the nutritional supplement with a desired flowability. This amount generally ranges from about 2 to about 95 weight percent of the total weight of the nutritional supplements, and preferably ranges from about 65 to about 90 weight percent, with about 78 weight percent being most preferred.

A nutritional supplement of the invention in the form of a food bar should not include the amount of water described above. In contrast, this food bar should have a moisture content that is about 10 weight percent of the total weight of the food bar or less, and that more preferably ranges from about 4 to about 5 weight percent (with a chewier food bar generally having a higher moisture content than a hard food bar).

Water may be employed, for example, to dissolve a vitamin pre-mix that may be employed to prepare nutritional supplements of the invention and/or to provide a liquid base for the nutritional supplements.

The temperature of the water employed to produce the nutritional supplements of the invention generally should be no higher than about 130° F. In addition, the water used should be in a liquid state (i.e., not frozen). Thus, the temperature of the water employed in processes within the invention should generally range from about ambient temperature to about 130° F., with ambient temperature being most preferred.

It is preferable that an 8-ounce serving of a ready-to-drink nutritional supplement of the invention contain about 185 ml of water.

### Other Optional Ingredients

It is contemplated that a wide variety of other edible ingredients may, optionally, be employed in the nutritional supplements of the invention. Such ingredients include any edible substance that is safe for consumption by humans, including diabetic, borderline diabetic and geriatric humans, pregnant or lactating women, women having childbearing potential that are attempting to become pregnant, and humans that have, are at risk for, or develop other glucose intolerance or cardiovascular disease, and are safe for developing fetuses and breast-fed babies of pregnant or lactating women, and which either alone, or in combination with one or more other ingredients, imparts a desirable taste, color, nutritional, medical and/or other health benefit to the nutritional supplements.

The amount and type of these optional ingredients that may be employed in the nutritional supplements of the invention may vary widely depending upon the particular ingredients that are chosen for use, the desired color, flavor, nutritional content and/or other medical and/or health benefit that is desired to be provided by the nutritional supplements, the form of the nutritional supplements (ready-to-drink liquid, liquid concentrate, solid concentrate, food bar, other solid form and the like), and upon the type, age, health and status of the humans that are to consume the nutritional supplements, and like considerations. Such an amount should be an amount that is effective for producing a desired result, and may readily be determined by those of ordinary skill in the art.

Examples of optional ingredients that may be incorporated into the nutritional supplements of the invention include, for example, vanilla or cocoa liquor, cocoa powder, mocha, caramel, yogurt, whole milk, skim milk, buttermilk, concentrated milk products (condensed or evaporated milk), dried milk products, nonfat milk powder, dry whole milk, modified whole milk, egg products, including egg whites and egg yolks, flour ingredients, such as wheat flour, rye flour, corn flour, cottonseed meal or sorghum flour, in either bleached or unbleached form, spices, such as cinnamon or nutmeg, salt, antioxidants, nuts, granola (whole grain rolled oats, rolled whole wheat, sugar, partially hydrogenated soybean and/or cottonseed oil, whole wheat flour, molasses, honey, cultured whey, soy lecithin and natural flavor), any of the components of granola, cereal, oats, wheat, corn, rice, rice crisp, soy, herbs, such as Ginko or Echinacea, sodium benzolate, para-aminobenzoic acid, inositol, lutein, vanadium, boron, nickel, silicon, tin, phytochemicals, isoflavonoids, essential and non-essential amino acids, bioflavonoids, such as quercetin and rutin NF, emulsifiers, diluents, edible organic acids and pieces of commercially-available or other candy or candy bars. All of these optional ingredients are commercially available from sources known by those of skill in the art and may be size reduced by known methods to obtain a desired bioavailability and/or palatability of the nutritional supplements.

Viscosity

The viscosity of the nutritional supplement of the invention in the form of a ready-to-drink liquid may vary widely, depending upon the nutrients that are present therein, the levels of nutrients present therein, processing aids such as emulsifiers, and like considerations, but preferably ranges from about 20 cp to about 45 cp, and more preferably ranges from about 30 cp to about 44 cp, and most preferably is about 38.2 cp. Viscosities of the nutritional supplements may be determined using a conventional viscometer.

Total Solids Content

The total solids content (amount of solids included) of the nutritional supplements of the invention in the form of a ready-to-drink liquid may also vary widely, depending upon the nutrients present therein, the levels of macronutrients and water present therein, and like considerations, and preferably ranges from about 15 to about 80 weight percent of the total weight of the nutritional supplements, and more preferably ranges from about 18 to about 25 weight percent, and most preferably is about 21.94 weight percent.

PH

The pH of the nutritional supplements of the invention preferably ranges from about 6.0 to about 7.0, and more preferably ranges from about 6.6 to about 7.0 (to achieve a maximum matrix stability), and may be adjusted using edible substances and methods known by those of skill in the art, such as citric acid (to decrease the pH) or sodium hydroxide (to increase the pH).

Caloric Content

The number of calories that a particular nutritional supplement of the invention may contain may vary widely depending upon a number of factors, such as the ingredients employed to produce the nutritional supplements, the quantities of those ingredients used, and the serving size chosen for the nutritional supplements. However, it is preferred that the nutritional supplements be low in calories.

A daily (24-hour period) quantity of the nutritional supplements of the invention, whether in a ready-to-drink liquid form, a food bar form, another solid form, or reconstituted from a liquid or solid concentrate, has a number of calories that preferably ranges from about 1,500 to about 2,500, and that more preferably ranges from about 2,000 to about 2,200.

One eight-fluid ounce serving of a ready-to-drink liquid nutritional supplement, or one 60-gram serving of a nutritional supplement food bar, contains a number of calories that preferably ranges from about 100 to about 400 calories per serving, and that more preferably ranges from about 200 to about 250 calories per serving, with about 220 calories per serving being most preferred. It is also preferred that such a serving of the nutritional supplements have about 70 of the calories being derived from oils and/or fat. In contrast, an 8-fluid ounce serving of Glucerna contains 80 calories derived from fat.

In the nutritional supplements of the invention, the percent of the total caloric value obtained from oils and/or fats preferably ranges from about 15% to about 45%, and more preferably ranges from about 28% to about 35%, with about 32% being most preferred. Such oils and/or fats are preferably present in a fat blend.

In the nutritional supplements of the invention, it is preferable that:
(a) about 10% or fewer of the total nutritional supplement calories are derived from saturated fat, with about 3% or fewer being more preferred;
(b) from about 5% to about 10% of the total nutritional supplement calories are derived from n-6 polyunsaturated fatty acids, with about 7% being more preferred;
(c) from about 15% to about 25% of the total nutritional supplement calories are derived from monounsaturated fat, with about 23% being more preferred; and
(d) about 3% or fewer of the total nutritional supplement calories are derived from trans fatty acids, with about 0.5% or fewer being more preferred.

The percent of the total caloric value of the nutritional supplements obtained from carbohydrates preferably ranges from about 30% to about 65%, and more preferably ranges from about 45% to about 55%, with about 51% being most preferred.

The percent of the total caloric value of the nutritional supplements obtained from proteins, including supplemented amino acids, preferably ranges from about 7% to about 30%, and more preferably ranges from about 13% to about 20%, with about 17% being most preferred. Such proteins are preferably present in a protein blend that includes sodium caseinate in an amount that preferably ranges from about 1.3 to about 2.5 weight percent of the total proteins, calcium sodium caseinate in an amount that preferably ranges from about 1.3 to about 2.5 weight percent of the total proteins, soy protein isolate in an amount that preferably ranges from about 0.6 to about 2.5 weight percent of the total proteins and added amino acids in an amount that preferably ranges from about 0.001 to about 2.0 weight percent of the total proteins.

The total calories of the nutritional supplements of the invention that are derived from a combination of proteins and carbohydrates is preferably less than about 80%, and more preferably less than about 75%, and still more preferably less than about 70%. Preferably, the total calories that are derived from proteins is about 17%, and the total calories that are derived from carbohydrates is about 51%.

It is preferable that the percent of the total caloric value of the nutritional supplements obtained from carbohydrates be from about 45% to about 75% greater than the percent of the total caloric value of the nutritional supplements obtained from oils and/or fats.

Preferably, the caloric distribution of the nutritional supplements of the invention comprises from about 20% to about 40% of oils and/or fats (combined), from about 40% to about 60% of carbohydrates (combined) and from about 10% to about 25% of proteins and/or amino acids (combined).

Particularly Preferred Embodiments

### Preferred Ready-to-Drink Liquid Nutritional Supplement

A particularly preferred embodiment of the ready-to-drink liquid form of the nutritional supplements of the invention is set forth below.

One serving size of the liquid nutritional supplement is 1 can (8 fluid ounces), which contains 220 calories (with 70 of the calories being from fat).

The liquid nutritional supplement contains the ingredients, and amounts thereof, and vitamins, minerals and elements described below.

| Ingredients | Formula % by Weight |
|---|---|
| Water | 78.060 |
| Maltodextrin | 5.870 |
| High Oleic Sunflower Oil | 2.440 |
| Crystalline Fructose | 2.420 |
| Maltitol Syrup | 2.810 |
| Sodium Caseinate | 1.690 |
| Calcium Sodium Caseinate | 1.690 |
| Soy Protein Isolate | 0.880 |
| Fructooligosaccharides | 0.440 |
| Vitamin/Mineral Mix | 0.790 |
| Fibersol2 - Maltodextrin | 0.320 |
| High Oleic Canola Oil | 0.330 |
| Gum Arabic | 0.240 |
| Cellulose Gum/Gel | 0.400 |
| Soy Lecithin | 0.170 |
| Potassium Chloride | 0.100 |
| Docosahexaenoic acid (DHA) | 0.230 |
| Potassium Citrate | 0.080 |
| Sodium Citrate | 0.080 |
| Carrageenan | 0.020 |
| Sucralose | 0.030 |
| Potassium Phosphate | 0.100 |
| Natural and Artificial French Vanilla Flavor | 0.250 |
| Natural and Artificial Vanilla Flavor | 0.100 |
| Tricalcium Phosphate | 0.100 |
| Magnesium Phosphate | 0.240 |
| Citric Acid | 0.120 |
| Total | 100% |

The Vitamin/Mineral Mix employed is the free-flowing powder product described hereinabove. The amino acids L-carnitine (0.0122 weight percent of the mix) and L-arginine are present in the Vitamin/Mineral Mix along with the vitamins and minerals described below.

Vitamins, Minerals and Elements

| | |
|---|---|
| Tricalcium Phosphate | Phytonadione |
| Cholecalciferol | Magnesium Phosphate |
| Ascorbic Acid | Potassium Iodide |
| Choline Chloride | Alphatocopheryl Acetate |
| Ferrous Sulfate | Zinc Sulfate |
| Niacinamide | Manganese Sulfate |
| D-Calcium Pantothenate | Copper Sulfate |
| Pyridoxine Hydrochloride | Vitamin A Palmitate |
| Thiamine Hydrochloride | Lutein |
| Riboflavin | Folic Acid |
| Beta Carotene | Biotin |
| Sodium Selenite | Sodium Molybdate |
| Cyanocobalamin | (38 mcg in an 8-ounce |
| Myoinositol (as Meso-Inositol) | serving) |
| (186 mg in an 8-ounce serving) | |
| Chromium Chloride | |
| (120 mcg in an 8-ounce serving) | |

This liquid nutritional supplement does not contain any taurine. It has a viscosity of about 38.2 cp and a total solids content of 21.94% by weight.

The liquid nutritional supplement has a “stabilizing system” that consists of a blend of two products sold by FMC. Inc.: (a) Avicel® CL-611 (Cellulose Gum/Gel); and (b) Seakem CM614 (Carrageenan). These two products help stabilize the nutritional beverage during thermal processes, provide mouthfeel to the nutritional beverage, suspend solids in the nutritional beverage and emulsify lipids. The nutritional supplement contains about 0.42% (about 1.06 grams) of this blend per serving, which equates to 4200 ppm on a % w/v basis or 1060 ppm on a % w/w basis. Gum arabic, which is also present in the nutritional supplement, also provides stabilization for the nutritional supplement, as well as general thickening properties.

Based upon a 2,000 calorie diet, the percent daily values of fat, cholesterol, sodium, potassium, total carbohydrate, dietary fiber and vitamins and minerals that are present in 1 can (8 fluid ounces) of the nutritional supplement are set forth below.

| Component | % Daily Value |
|---|---|
| Total Fat | 13% |
| Saturated Fat | 3% |
| Trans Fat | 0% |
| Cholesterol | 0% |
| Sodium | 7% |
| Potassium | 10% |
| Total Carbohydrate | 10% |
| Dietary Fiber | 13% |
| Vitamin A | 35% |
| Calcium | 25% |
| Vitamin D | 25% |
| Vitamin K | 25% |
| Riboflavin | 25% |
| Vitamin B6 | 50% |
| Vitamin B12 | 50% |
| Pantothenic Acid | 25% |
| Iodine | 25% |
| Zinc | 25% |
| Copper | 25% |
| Chromium | 100% |
| Chloride | 10% |
| Vitamin C | 100% |
| Iron | 25% |
| Vitamin E | 100% |
| Thiamin | 25% |
| Niacin | 25% |
| Folate | 100% |
| Biotin | 25% |
| Phosphorus | 30% |
| Magnesium | 25% |
| Selenium | 25% |
| Manganese | 50% |
| Molybdenum | 50% |

The nutritional supplement contains the following weight percents of proteins, carbohydrates and fats:
(a) about 4% protein;
(b) about 12% carbohydrate (in the form of a carbohydrate blend); and
(c) about 4% fat (in the form of a fat blend).

The carbohydrate blend that is employed in the nutritional supplement contains the following amounts (on a percent basis of individual carbohydrates) of the carbohydrates indicated below:
(a) about 49% maltodextrin (10DE);
(b) about 23% crystalline fructose;
(c) about 20% maltitol;
(d) about 3% maltodextrin (digestive resistant dextrin—Fibersol 2);
(e) about 2% gum arabic; and
(f) about 3% cellulose gel.

A fat blend that is employed in the nutritional supplement contains the following amounts (on a percent basis of individual fats) of the types of fats indicated:
(a) about 9% of saturated fat;
(b) about 69% of monounsaturated fat; and
(c) about 22% of polyunsaturated fat.

The percent of the total caloric value obtained from protein in the nutritional supplement is about 17%. The percent of the total caloric value obtained from the carbohydrate blend is about 51%. The percent of the total caloric value obtained from the fat blend is about 32%. The percent of the total formula calories derived from saturated fat is about 3%. The percent of the total formula calories derived from polyunsaturated fat is about 5%. The percent of the total formula calories derived from trans fatty acids is less than about 0.5%. The percent of the total formula calories derived from monounsaturated fatty acids is about 23%. The dextrose equivalence of starch employed is 10. To achieve a maximum matrix stability, the pH of the formula is preferably adjusted to between about 6.6 and 7.0 using either citric acid or sodium hydroxide.

### Nutritional Supplements for Pregnant or Lactating Women

In a preferred embodiment, the nutritional supplements may be employed to produce pleasant tasting, nutritional supplements for the oral consumption by diabetic or borderline diabetic pregnant women, lactating women (women who are secreting milk) or women having childbearing potential that are attempting to become pregnant containing one or more vitamins, minerals and/or other nutrients recommended for consumption by pregnant women, lactating women or women having childbearing potential that are attempting to become pregnant, preferably including calcium in an amount above 1,000 mg, as well as one or more optional taste-masking agents (to mask the taste of unpleasant tasting ingredients) and one or more optional anti-constipation and regularity-maintaining agents. Infertile women (women of childbearing age that have unsuccessfully attempted to become pregnant for more than one year, but that have the capacity to become pregnant) are one example of women having childbearing potential that are attempting to become pregnant. Other examples of such women include women of childbearing age that have the ability to become pregnant, and that have unsuccessfully attempted to become pregnant for one year or less, or that are commencing their attempts to become pregnant.

Nutritional supplements prepared in accordance with the methods of the invention that are for consumption by pregnant women, lactating women or women having childbearing potential that are attempting to become pregnant preferably, but optionally, comprise one or more vitamins and/or minerals recommended for consumption by pregnant women, lactating women or women having childbearing potential that are attempting to become pregnant in an amount that is effective for enhancing the nutrition of pregnant women, lactating women or women having childbearing potential that are attempting to become pregnant, or of their developing fetuses or babies, and that is not harmful to developing fetuses or breast-fed babies, DHA in an amount that is effective for providing, or increasing the level of, DHA in pregnant women, lactating women or women having childbearing potential that are attempting to become pregnant, or in their developing fetuses or babies (through, for example, the placenta, or the mothers' breast milk), and that is not harmful to developing fetuses or breast-fed babies, one or more taste-masking agents in an amount that is effective for masking the taste of one or more unpleasant tasting ingredients present in or on the nutritional supplements, and that is not harmful to developing fetuses or breast-fed babies, and one or more anti-constipation and regularity-maintaining agents in an amount that is effective for maintaining regular bowel movements and/or reducing or eliminating constipation, and that is not harmful to developing fetuses or breast-fed babies.

Use of Nutritional Supplements

The nutritional supplements of the invention are suitable for use, and may be safely and freely consumed without regard to special conditions, by the general population of humans, including each of the sexes of all age groups that are 4 years of age or older, and are particularly beneficial for use by the borderline diabetic, diabetic, otherwise glucose intolerant and geriatric population, as well as by humans that have cardiovascular disease.

Depending upon the circumstances, upon the form of the nutritional supplement employed, and upon the health and medical condition of the human to receive a nutritional supplement, the nutritional supplements of the invention may be orally or enterally administered to humans. For example, a diabetic human that is in a coma, or that has a severe injury to the face, could have a nutritional beverage of the invention administered enterally. In contrast, a diabetic human that does not have these medical issues could orally consume the nutritional supplements.

The nutritional supplements of the invention are preferably used under the supervision and direction of a physician or dietician, for example, as a nutritional or dietary supplement, a snack between meals and/or as a meal replacement (i.e., as a meal replacement food product that is consumed in place of a meal, such as breakfast, lunch, dinner or the like).

Depending upon the specific composition of a particular nutritional supplement within the invention, the nutritional supplement may or may not have an ability to serve as the sole source of nutrition for some humans (i.e., they may or may not be nutritionally complete and, thus, may or may not be able to provide 100% of the daily requirements of all of the nutrients required by humans for complete nutrition). Depending upon their objectives, and the desired route of administration (orally or enterally), those of skill in the art can produce nutritional supplements of the invention that are, or are not, nutritionally complete.

The nutritional supplements of the invention may be used on either a short-term basis, for example, from a single meal replacement to exclusive use for 4 consecutive meals, or on a long-term basis (depending upon directions of a health care provider), for example, up to about one month, and in combination with traditional pharmaceutical con-commitment insulin or other therapy. For humans that are in satisfactory health, the nutritional supplements are preferably used only on a short-term basis.

For humans that are in satisfactory health, the nutritional supplements are preferably used only on a short-term basis (i.e., about one month or less). Short-term use might be prescribed or recommended by a physician as part of a control program to stabilize weight or glycemic index. For example, a physician might prescribe nutritional supplements within the invention to be used in concert with a prescribed (controlled) diet in order to establish long-term insulin needs. Thereafter, long-term use (more than about one month, such as for an entire lifetime of a human) of the nutritional supplements could safety be used by a human patient with an ad libum diet within the context of established insulin needs. Long-term use of nutritional supplements of the invention is generally safe for humans because the nutritional supplements generally contain nutritional ingredients that are safe for consumption by humans in a unique formulation that is heart healthy and otherwise beneficial.

Although the nutritional supplements of the invention are safe for general consumption, as a result of the serious nature of borderline diabetes, diabetes, cardiovascular disease and other disease conditions that could benefit from these supplements, they should be used in consultation with professional health care supervision and as an adjunct to appropriate medical intervention.

Prior to use, ready-to-drink liquid nutritional supplements within the invention that are to be consumed orally are preferably chilled and shaken well (so that nutritional components are not “settled out” of the supplements). Unused portions of the nutritional supplements which, for example, may be present in a can or bottle, are preferably covered and refrigerated. Such ready-to-drink nutritional supplements may be employed without chilling when administered to a human enterally.

Ready-to-drink liquid nutritional supplements of the invention that are to be administered to a human parenterally preferably have their viscosities reduced by at least about 80%. This can be accomplished, for example, according to methods and techniques known by those of skill in the art, and using sterile water and ph buffer (for neutralization).

Blood Glucose Responses

The nutritional supplements of the invention generally induces a blood glucose response in a mammalian diabetic patient that ranges from about 45 to about 65 mg/dL, and that more usually ranges from about 50 to about 60 mg/dL, within a period of time that generally ranges from about 4 to about 45 minutes, and that more usually ranges from about 20 to about 30 minutes, and that lasts for a period of time that generally ranges from about 30 to about 180 minutes, and that more usually ranges from about 60 to about 120 minutes.

Blood glucose responses can be determined using known methods and commercially available equipment, such as the Precision™ blood glucose monitors (Abbott Laboratories, Abbott Park, Ill.) and blood glucose monitors distributed by the DiabetesStore.com (Pompano Beach, Fla.).

Form of Nutritional Supplements

The nutritional supplements of the invention may be in the form of a ready-to-drink beverage, such as a shake or drink, a liquid concentrate that may be diluted with water (or another liquid), a powder (a dry blend and/or agglomeration for mixing with water, or another liquid), a solid, such as a nutritional bar (in a snack or meal-replacement dosage).

Methods of Production

The nutritional supplements of the invention may readily be prepared in a cost-effective manner using machinery and techniques that are known by those of skill in the art for preparing nutritional supplements. General methods, conditions and equipment for producing a wide variety of different nutritional products, and flavors therefore, are well known by those of skill in the art.

Methods for the production of nutritional supplements are described in detail herein.

The process described below is unique with respect to the order of ingredient addition, as well as the manner whereby multiple (more than one) ingredients are formed into a resultant stable formula matrix. However, the steps and conditions set forth in the methods described herein may be varied in a manner known by those of skill in the art to produce nutritional supplements within the invention, or other processes may be employed to produce the nutritional supplements of the invention.

Any of a wide variety of known mixing techniques and/or equipment can be employed to produce each of the mixtures described herein.

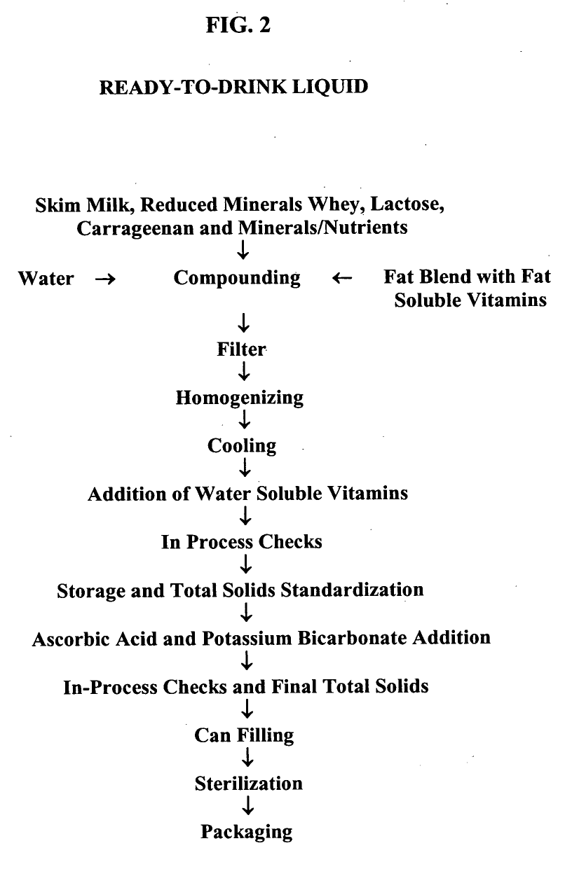

FIG. 2 is a schematic diagram that shows one embodiment of a process for preparing ready-to-drink liquid nutritional supplements according to the invention.

A preferred procedure for preparing ready-to-drink nutritional beverages of the invention employs the steps described below. All ingredients employed in the nutritional beverages are preferably pre-weighed using conventional weighing processes and equipment.
(a) An amount of water that is effective for an appropriate hydration of ingredients and a creation of a working solution is heated to a temperature that is effective for enhancing the formation of a solution of ingredients using conventional heating equipment, such as a steam-jacketed kettle. Such temperature generally ranges from about 155° F. to about 160° F., and is maintained during this step of the process, but is then reduced or eliminated until sterilization in a retorted liquid. The amount of water employed may vary widely, depending upon the quantity of the nutritional supplements being prepared, the viscosity desired and like considerations. Those of skill in the art can readily vary the amount of water employed to meet specific objectives for the nutritional supplements. As the water is heated to the desired temperature, the water is agitated or otherwise mixed using conventional methods and equipment, such as mechanical, electrical or manual stirring equipment.
(b) An amount of agents that function as a carbohydrate contributor or sweetener, such as maltitol and Sucralose, and that is effective for providing a nutritional or sweetening effect, is added to, and blended with, the water for a period of time, and at a level of agitation, that is sufficient to uniformly distribute these ingredients within the water. Such an amount of time, and level of agitation, can vary widely depending upon the quantity of the nutritional supplements being prepared, the viscosity desired and like considerations, but generally ranges from about 10 to about 30 minutes at a level of agitation that ranges from about 10 rpm to about 60 rpm.
(c) An amount of mineral salts and/or buffering agents, such as dipotassium phosphate, magnesium phosphate, potassium citrate, sodium citrate, potassium chloride and citric acid that is effective for providing a desired mineral content and/or for pH buffering, is dry blended together using conventional equipment, such as a ribbon blender, until a uniform blend of these ingredients is produced, which generally takes about 10 to 60 minutes, and added slowly to the mixture of step (b) (at a rate that prevents clumping or concentration and that facilitates homogenicity, which should take no longer than about 10 minutes depending upon the conditions and size of batch), and mixed with the liquid blend for a period of time, and at a level of agitation, that is sufficient to permit the various ingredients to disperse and hydrate. Such an amount of time, and level of agitation, can vary widely depending upon the quantity of the nutritional supplements being prepared, the viscosity desired and like considerations, but generally ranges from about 1 to about 3 minutes at a level of agitation that ranges from about 10 rpm to about 30 rpm.
(d) An amount of functional formula excipients or ingredients, such as cellulose gum/gel, carrageenan, gum arabic, fructooligosaccharide and Fibersol 2 maltodextrin, that is effective for providing desired matrix characteristics and/or nutritional content is dry blended with other compatible ingredients, such as M-100 maltodextrin, using conventional equipment until a uniform blend of these ingredients is produced, which generally takes from about 3 to about 10 minutes. The resulting dry blend is added to, and mixed with, the liquid blend for a period of time, and at a level of agitation, that is sufficient to permit the various ingredients to disperse and hydrate. Such an amount of time, and level of agitation, can vary widely depending upon the quantity of the nutritional supplements being prepared, the viscosity desired and like considerations, but generally ranges from about 1 to about 5 minutes at a level of agitation that ranges from about 5 rpm to about 30 rpm.
(e) An amount of fats and/or oils, such as high oleic sunflower oil and high oleic canola oil, that is effective for providing a desired level of fatty acids, an amount of DHA (preferably that is high grade, and is obtained from a source other than an aquatic animal) and that is effective for providing, or enhancing, a nutritional, medical or other health benefit, and an amount of emulsifiers, such as soy lecithin, that is effective for assisting in the proper mixing of aqueous and lipid materials, are added to the liquid blend with agitation.
(f) An amount of food ingredients, such as tricalcium phosphate, that is effective for providing both calcium and phosphorus nutritional content to the nutritional supplements, and an amount of a vitamin and mineral premix that is effective for providing a desired nutrient content to the nutritional supplements, are dry blended together using conventional equipment until a uniform blend of these ingredients is produced, which generally takes from about 1 to about 60 minutes minutes. The dry blend is added slowly to the liquid blend of step (e) (at a rate that is sufficient to avoid clumping or concentration, and that enhances homogenicity, and mixed with the liquid blend for a period of time, and at a level of agitation, that is sufficient to permit the various ingredients to disperse and hydrate. Such an amount of time, and level of agitation, can vary widely depending upon the quantity of the nutritional supplements being prepared, the viscosity desired and like considerations, but generally ranges from about 5 to about 60 minutes at a level of agitation that ranges from about 5 rpm to about 60 rpm.
(g) An amount of flavorings that are effective for providing a flavor to the nutritional beverages is added to, and mixed with, the liquid blend of step (f) until a uniform blend of these ingredients is produced, which generally takes about 1 to 20 minutes.
(h) The pH of the liquid blend produced in step (g) is determined and, if necessary, adjusted either by adding an amount of an acid, such as 1N citric acid, or an amount of a base, such as 1N sodium hydroxide, thereto that is effective to adjust the pH of the liquid blend to a level ranging from about 6.6 to about 7.0.
(i) The temperature of the liquid blend of step (h) is determined and, if necessary, adjusted to a temperature that is effective for providing a more efficient homogenization, and helping to mitigate undue microbiologic growth, which generally ranges from about 155° F. to about 160° F.
(j) The mixture of step (i) is homogenized using standard homogenization processing and equipment for a period of time, and at a pressure, that is effective for homogenization, which generally ranges from about 10 seconds to about 3 minutes at a pressure ranging from about 2,000 to about 2,800 psi.
(k) The mixture of step (j) is filled into appropriate retort compatible containers (according to the size of the batch and equipment employed) and processed using conventional retort processes and equipment. (Retort products are generally filled into their container, which traditionally has been an aluminum can or multi-layer foil pouch. Once sealed, containers are heated to extremely high temperatures, rendering the product sterile, with bacteria being destroyed.)

Those of ordinary skill in the art will recognize that the times, rates of agitation and other variables described above may vary depending upon the particular mixing equipment employed, the quantity of the nutritional beverages being produced and like considerations.

Those of skill in the art will also recognize that other methods may be utilized to prepare the nutritional beverages of the invention, and that the temperatures, pressures, times, agitation rates and order of steps employed in preparing the nutritional beverages may be varied.

Irradiation

Prior to packaging, nutritional supplements of the invention in the form of food bars may, optionally, be irradiated in order to kill microorganisms that may have contaminated the nutritional supplements during their production. Such irradiation should not diminish the effectiveness of the vitamins, minerals, elements and other nutrients and beneficial ingredients that may be contained therein.

Packaging

The nutritional supplements of the invention may be packaged in any manner known by those of skill in the art for packaging food products, and in any convenient or appropriate containers or forms.

A preferred method for packing nutritional supplements in the form of food bars or powders is by hermetically sealing the products within a film having oxygen barrier properties, such as a foil laminate, purging the inner space of the wrapper with nitrogen (to create a nitrogen blanket), placing the nutritional supplements in an aluminum (or other metal), glass or plastic container, such as a can or bottle, having the air present in the container removed with a vacuum, and replacing the removed air with nitrogen.

The nutritional supplements of the invention in the form of ready-to-drink liquids may, for example, be packaged in a manner such that one package of the nutritional supplements contains six eight-fluid ounce cans containing the nutritional supplements. Retorted product would generally be present in hermetically sealed metal, plastic or other material that is sufficient to safely withstand retortings and thermal stress, and that will not insult intended product specifications.

Methods for Measuring Oxidation

The amount of oxidation, degradation and/or decomposition that may occurr to an edible fat or oil, or to a solid or liquid food product containing one or more edible fats or oils, can be determined by tests known by those of skill in the art.

The most commonly used measure for testing an oxidation of an edible fat or oil, or of a solid or liquid food product containing one or more edible fats or oils, is the Peroxide Value Test, which measures the concentration of the immediate products of oxidation (peroxides). However, these initial peroxide products are themselves degraded over time to various aldehydes, and these aldehyde secondary products are not detected by the Peroxide Value Test. A second known test, the Anisidine Test, may be used to detect the secondary products. Thus, the Peroxide Value Test measures the degree to which oxidation is taking place at the present moment, and the Anisidine Test measures the amount of historical oxidation over the life of a edible fat, oil or food product. These two measures may be combined to give a total oxidation (or TOTOX) value, which is calculated as:
TOTOX=Anisidine Value+(2×Peroxide Value).
Further, known organoleptic testing procedures can be employed to test the taste and smell of edible fats and oil, and food products containing one or more edible fats and/or oils.

Quantities to be Consumed or Administered

The amount of nutritional supplements that may be consumed by, or administered to, a human in a 24-hour period may generally be an amount that is not toxic to the human, taking into consideration the age, condition and health of the human, as well as the human's insulin or other tolerance for carbohydrate intake, and levels of vitamins, minerals and other nutrients that are safe for consumption by the human, and safe for developing fetuses and breast-fed babies of lactating humans, all of which should be known, or can be readily determined, by those of skill in the art, such as a physician, dietician or other healthcare provider.

A typical dose (serving) of the ready-to-drink liquid form of the nutritional supplements of the invention is one 8-fluid ounce serving. A typical dose of a food bar form of the nutritional supplements of the invention is one 60-gram serving. Preferably, from about 1 to about 3 of such 8-fluid ounce or 60-gram servings (or a combination of the two types of servings) will be administered to a human in one 24-hour period.

The dosages described herein for the nutritional supplements of the invention, administered on a continuous basis, should be sufficient to produce the beneficial results of the various methods described herein.

If one 8-fluid ounce or 60-gram serving (or other size) of a nutritional supplement of the invention is prepared that contains all of the vitamins, minerals, elements, nutritional and/or other ingredients, agents and/or supplements that are described herein in the largest daily quantity that is safe for consumption by humans, including diabetic, borderline diabetic or geriatric humans, pregnant or lactating women, humans that have, are at risk for, or develop other glucose intolerance or cardiovascular disease or woman of childbearing potential that are attempting to become pregnant, and is safe for developing fetuses or breast-fed babies of pregnant or lactating women, one serving of the nutritional supplements would preferably be consumed or administered per day (either all at one time, or divided into two, three, four, five, six, seven, eight or more equal or non-equal portions).

If one 8-fluid ounce or 60-gram serving (or other size) of a nutritional supplement of the invention is prepared that contains all of the vitamins, minerals, elements, nutritional and/or other ingredients, agents and/or supplements that are described herein in one half of the largest daily quantity that is safe for consumption by humans, and is safe for developing fetuses or breast-fed babies of pregnant or lactating women, two servings of the nutritional supplements would preferably be consumed or administered per day.

Using information that is provided herein, and information that is known, those of ordinary skill in the art can readily determine the quantity of a nutritional supplement of the invention that should preferably be consumed or administered per day (or per another period of time) for a diabetic, borderline diabetic or geriatric human, a pregnant or lactating woman, a human that has, is at risk for, or develops other glucose intolerance or cardiovascular disease, a woman of childbearing potential that is attempting to become pregnant or another human. Those of skill in the art can readily determine the appropriate daily (or other) dosage of nutritional supplements of the invention in relation to the type and quantity of vitamins, minerals, elements, nutritional and/or other ingredients, agents and/or supplements that may be present in the nutritional supplements, and to the type, age, health and status of a human that is to consume the nutritional supplement.

For pregnant women that experience morning sickness, it would be preferable to consume several smaller sized servings, for example, one-, two-, three- or four-fluid ounce or 15-gram servings, of a nutritional supplement of the invention throughout the day than to consume one or more larger sized servings of the nutritional supplement all at one time during a day.

Liquid and Solid Concentrates

The present invention additionally provides a liquid concentrate nutritional supplement for an administration to humans comprising a ready-to-drink liquid nutritional supplement of the invention, wherein an amount of water has been removed from the ready-to-drink liquid nutritional supplement in a manner that is effective for forming a liquid concentrate nutritional supplement from the ready-to-drink liquid nutritional supplement, and wherein the liquid concentrate can be transformed into a ready-to-drink liquid nutritional supplement by an addition of an amount of water that is effective for transforming the liquid concentrate into a ready-to-drink liquid nutritional supplement.

The present invention also provides a solid concentrate nutritional supplement for an administration to humans comprising a ready-to-drink liquid nutritional supplement of the invention, wherein an amount of water has been removed from the ready-to-drink liquid nutritional supplement in a manner that is effective for forming a solid concentrate nutritional supplement from the ready-to-drink liquid nutritional supplement, and wherein the solid concentrate can be transformed into a ready-to-drink liquid nutritional supplement by an addition of an amount of water that is effective for transforming the solid concentrate into a ready-to-drink liquid nutritional supplement.

Preferably, from about 25 to about 65 weight percent, and more preferably, from about 35 to about 60 weight percent, of the water that is present in a ready-to-drink liquid nutritional supplement of the invention will be removed from the ready-to-drink liquid nutritional supplement in order to produce a liquid or solid concentrate of the ready-to-drink liquid nutritional supplement. However, this amount may vary widely depending upon the amount of water that is present in the ready-to-drink liquid nutritional supplement, the quantity of the ready-to-drink liquid nutritional supplement being concentrated and like considerations, and may readily be determined by those of skill in the art.

Evaporation is a process of heating liquid to the boiling point to remove water as vapor. Commercially available evaporators include batch pan, rising film and falling film evaporators.

Dehydration refers to the nearly complete removal of water from liquid or solid foods to a level that is generally less than about 5%. Although there are many types of driers, spray driers are the most widely used type of air convention drier. Drying typically occurs within a matter of seconds at temperatures of about 200° C.

In spray-drying processes, a liquid food or beverage is generally preconcentrated by evaporation to economically reduce the water content. The resulting concentrate is then generally introduced as a fine spray or mist into a tower or chamber with heated air. As the resulting small droplets of the concentrate make contact with the heated air, they generally flash off their moisture, become small particles, drop to the bottom of the tower and are removed.

Those of skill in the art can readily form nutritional supplements of the invention in the form of liquid or solid concentrates from nutritional supplements of the invention in the form of ready-to-drink liquids using conventional concentrate, evaporation, dehydration, spay-drying, freeze drying, chemical drying, agglomeration, powdering, milling, grinding and/or other size-reducing processes and equipment.

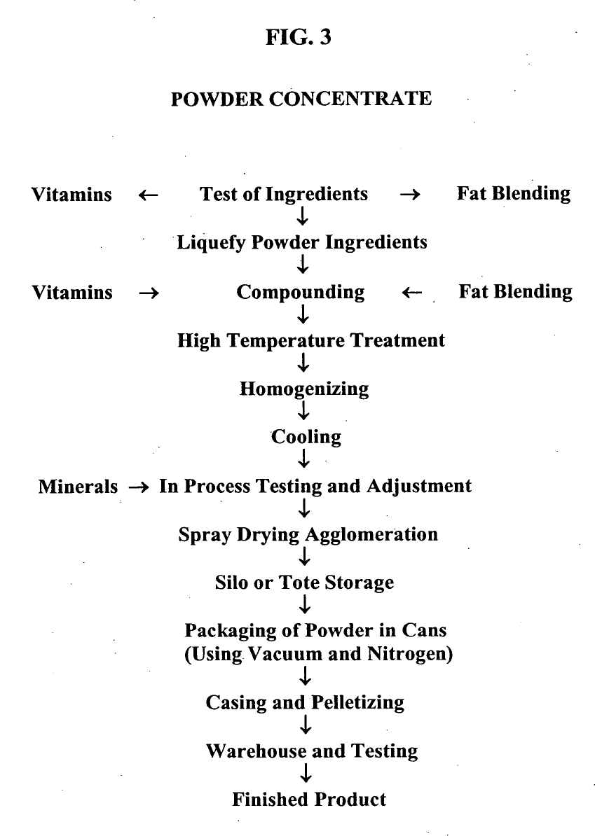

A spray-dried powder can be produced, for example, from a ready-to-drink liquid or liquid concentrate form of the nutritional supplements of the invention using conventional spray drying processes and equipment to spray-dry the ready-to-drink liquid or liquid concentrate form of the nutritional supplement. The resulting solid, powdered or granular product may be reconstituted in an aqueous media to exhibit the same, or similar, characteristics as the ready-to-drink liquid or liquid concentrate form of the nutritional supplements. FIG. 3 is a schematic drawing showing one embodiment of the process for preparing a spray dried concentrate nutritional supplements according to the invention.

In a similar manner, an agglomerated powder can be produced from a ready-to-drink liquid or liquid concentrate form of the nutritional supplements of the invention using conventional agglomeration processes and equipment. Prior to spray drying or agglomeration, the nutritional beverages may be homogenized or subjected to high shear mixing using conventional processes and equipment. Spray drying, freeze-drying, chemical drying and concentrating equipment is commercially available, for example, from Niro A/S (Soeborg, Denmark).

When a solid concentrate is produced from a ready-to-drink liquid or liquid concentrate form of a nutritional supplement of the invention, solid powder or other particles resulting from such production process may vary widely, and depend upon the particular process and equipment employed to produce the solid particles, the particular composition of the ready-to-drink or liquid concentrate nutritional supplement employed and like considerations. The size of the particles may be of any size that permits them to be in the form of a solid concentrate, and to be reconstituted into a ready-to-drink liquid, which may readily be determined by those of skill in the art. Preferably, the solid concentrate is in a free-flowing form, such as a free-flowing powder, and is unclumped (contains few or no clumps).

Nutritional supplements of the invention in the form of a liquid or solid concentrate may readily be re-constituted or diluted back to their original state, or to a similar state, with an addition of water (or another base component or solvent) by a user. The amount of water to be added will generally be about the same amount, or a similar, amount as the amount of water that had been removed from the ready-to-drink liquid when producing a liquid or solid concentrate, and may readily be determined by those of skill in the art.

The formation of liquid or solid concentrates via a removal of water from liquid foods or beverages advantageously can provide microbiological stability to the products, reduce deteriorative chemical reactions and reduce transportation and storage costs (by reducing the weight of products being transported). The liquid or solid concentrates can readily be re-constituted at the time of usage by a user via an addition of solvent to the concentrate.

Liquid and powder concentrates are generally an ideal format for use in ready-to-drink beverage production.

Food Bars

General methods for preparing food (nutrition) bars are described in detail in U.S. Pat. No. 6,569,445 B2 and U.S. Pat. No. 6,576,253 B2.

The nutrition bars and other solid nutritional supplements of the invention can be prepared using techniques that are well-known in the art for preparing such food products, such as cold-forming and partially cold-forming techniques.

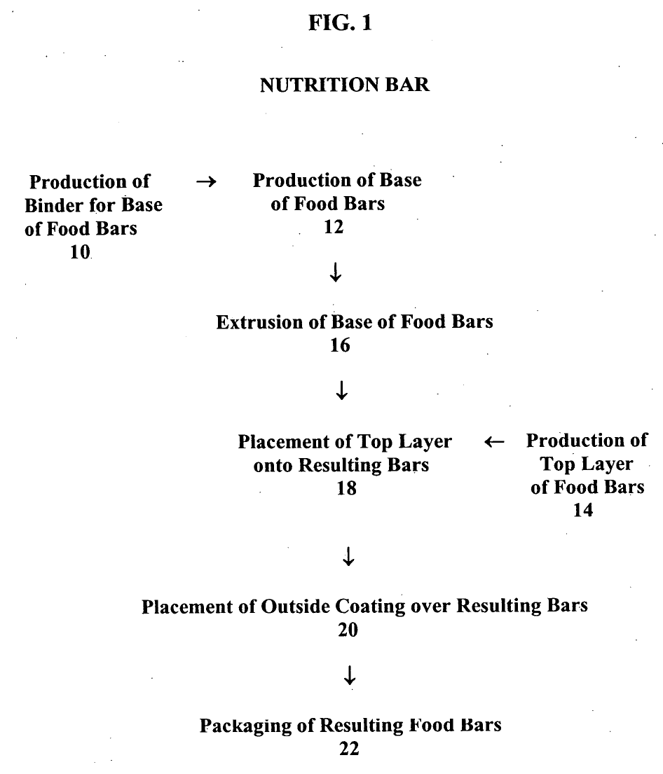

FIG. 1. depicts a preferred general process for preparing food bars. Referring to FIG. 1, separately, a binder for the base of the food bars is produced 10, the base of the food bars is produced 12, and a top layer for the food bars is produced 14. The base of the food bars is then extruded 16 to produce bars of the desired shape and size. A top layer is then placed on top of each of the extruded bars 18, and the resulting bars are coated on the top, bottom and all sides with an outside coating 20. The resulting food bars of the invention are then packaged 22.

Nutritional supplements within the invention in the form of food bars can be prepared in the manner shown in FIG. 1, and described in U.S. Pat. No. 6,569,445 B2 and U.S. Pat. No. 6,576,253 B2, with the exception that the specific ingredients, and quantities thereof, are included within these food bars, and that the limitations described herein are applied to these food bars. Those of skill in the art can readily employ the information that is present herein, in FIG. 1, and in U.S. Pat. No. 6,569,445 B2 and U.S. Pat. No. 6,576,253 B2 to produce nutritional supplements of the invention that are in the form of food bars (or other solid food products).

Methods

The present invention also provides a method for providing one or more nutritional, medical or other health benefits to a human comprising administering to the human a nutritional supplement of the invention in an amount that is effective for providing one or more nutritional, medical and/or other health benefits to the human, and for a period of time that is necessary or desirable for providing one or more nutritional, medical and/or other health benefits to the human.

The present invention further provides a method for maintaining, or aiding in the maintenance of, a normal blood glucose level of a diabetic, borderline diabetic or other human that has glucose intolerance comprising administering to the human a nutritional supplement of the invention in an amount that is effective for maintaining, or aiding in the maintenance of, the blood glucose level of the human at a normal level, and for a period of time that is necessary or desirable for maintaining, or aiding in the maintenance of, the blood glucose level of the human at a normal level.

Additionally, the present invention provides a method for returning, or aiding in the return of, an abnormal blood glucose level of a diabetic, borderline diabetic or other human that has glucose intolerance to a normal blood glucose level comprising administering to the human a nutritional supplement of the invention in an amount that is effective for returning, or aiding in the return of, the blood glucose level of the human to a normal level, and for a period of time that is necessary or desirable for returning, or aiding in the return of, the blood glucose level of the human to a normal level.

The present invention further provides a method for preventing, or reducing the likelihood of, a pre-diabetic or borderline diabetic condition of a human from transforming into diabetes or another glucose intolerance disorder comprising administering to the human a nutritional supplement of the invention in an amount that is effective for preventing, or reducing the likelihood of, the human from having a pre-diabetic or borderline diabetic condition transforming into diabetes or another glucose intolerance disorder, and for a period of time that is necessary or desirable for preventing, or reducing the likelihood of, the pre-diabetic or borderline diabetic condition transforming into diabetes or another glucose intolerance disorder.

The present invention additionally provides a method for preventing, or reducing the likelihood of, a diabetic, borderline diabetic or human that has glucose intolerance from experiencing one or more symptoms or complications related to, or produced by, diabetes or glucose intolerance comprising administering to the human a nutritional supplement of the invention in an amount that is effective for preventing, or reducing the likelihood of, the human from experiencing such symptoms or complications, and for a period of time that is necessary or desirable for preventing, or reducing the likelihood of, the human from experiencing such symptoms or complications.

The present invention also provides a method for preventing, or reducing the likelihood of, a human from having pre-mature cardiovascular disease comprising administering to the human a nutritional supplement of the invention in an amount that is effective for preventing, or reducing the likelihood of, a human from having pre-mature cardiovascular disease, and for a period of time that is necessary or desirable for preventing, or reducing the likelihood of, a human from having pre-mature cardiovascular disease.

The following examples describe and illustrate the methods for the preparation of the food products of the present invention. The examples are intended to be merely illustrative of the present invention, and not limiting thereof in either scope of spirit. Those of skill in the art will readily understand that variations of certain of the conditions and/or steps employed in the procedures described in the examples can be used to prepare and test these food products.

Sources of Ingredients

All of the ingredients, materials and equipment employed in the examples, and generally employed to produce nutritional supplements of the invention, are commercially available from sources known by those of skill in the art, such as those sources described hereinabove, as well as Kraft Foods, Inc. (Northfield, Ill.), Orafti Active Food Ingredients (Malvern, Pa.), Cargill, Incorporated (Minneapolis, Minn.), BASF Corp. (Mt. Olive, N.J.), RFI Ingredients (Blauvelt, N.Y.), Hoffmann-LaRoche Inc. (Nutley, N.J.), Flavor-Savor, Inc. (Franklin Park, Ill.), Grain Processing Corporation (Muscatine, IO), Debelis Chocolate Ingredients (Kenosha, Wis.), Martek Corp. (Columbia, Md.), Ocean Nutrition Canada, Ltd. (Bedford, Nova Scotia, Canada), Pronova Biocare AS (Lysakar, Norway), Napro AS (Brattvaag, Norway), DSM (Heerlen, NL), Berg Lipotech AS (Norway), ADM Food Oils (Decatur, Ill.), Oilseeds International, Ltd. (San Francisco, Calif.) and The Edible Oils Co. (www.edible oils.net).

### EXAMPLE 1

### Preparation of a Vanilla-Flavored Ready-to-Drink Nutritional Beverage

This example describes the preparation of a vanilla-flavored ready-to-drink nutritional beverage of the invention, which is packaged into 8-fluid ounce servings. Safe varieties and ranges of the ingredients employed in this example are +/−40% of the carbohydrate and protein complex, as well as the amino acids and nutrients such as DHA, L-arginine, L-carnitine, folate, ascorbic acid, vitamin E, chromium, iron, copper, beta-carotene and fat.

The ingredients, sources thereof, code or special characteristics and weight percents of formula are set forth below.

| | | Code or Special | Formula % |
|---|---|---|---|
| Ingredient | Source | Characteristic | by Weight |
| Water | Water Supply | Filtered | 78.060 |
| Maltodextrin | Grain Processing Corporation | M-100 | 5.870 |
| | (Muscatine, Iowa) | | |
| High Oleic | Cargill Specialty Oils | Sunflower Oil | 2.440 |
| Sunflower Oil | (Port Klang, Klang, Selangor) | | |
| Crystalline | Farbest Brands, Inc. | Krystar 300 | 2.420 |
| Fructose | (Yardly, PA) | | |
| Maltitol | Pacific Coast Chemicals, Co. | M-95 | 2.810 |
| Syrup | (Berkeley, CA) | | |
| Sodium | Fonterra/NZMP | Alanate 180 | 1.690 |
| Caseinate | (Wellington, New Zealand) | | |
| Calcium Sodium | Fonterra/NZMP | Alanate 220 | 1.690 |
| Caseinate | (Wellington, New Zealand) | | |
| Soy Protein | The Solae Company | XT30N | 0.880 |
| Isolate | (St. Louis, Missouri) | | |
| Fructo- | Pacific Coast Chemicals, Co. | PTX Complete Inulin | 0.440 |
| Oligosaccharide | (Berkeley, CA) | | |
| Vitamin/ | Fortitech, Inc. | FT041280 | 0.790 |
| Mineral Mix | (Schenectady, NY) | | |
| Maltodextrin | Matsutani America, Inc. | Fibersol 2 | 0.320 |
| | (Decatur, Illinois) | | |
| High Oleic | Cargill Specialty Oils | CV65 | 0.330 |
| Canola Oil | (Port Klang, Klang, Selangor) | | |
| Gum Arabic | TIC Gums | Pretested FT Powder | 0.240 |
| | (Belcamp, MD) | | |
| Cellulose Gum/ | ET Horn/FMC Corporation | Avicel CL611 | 0.400 |
| Gel | (Philadelphia, PA) | | |
| Soy Lecithin | SPI Group | Centrolex F | 0.170 |
| | (San Leandro, CA) | | |
| Potassium Chloride | Vivion, Inc. | Anhydrous | 0.100 |
| | (San Carlos, CA) | | |
| Docosahexaenoic | Martkek Biosciences | DHASCO-S | 0.230 |
| Acid (DHA) | (Columbia, MD) | | |
| Potassium Citrate | Pacific Coast Chemicals, Co. | Anhydrous | 0.080 |
| | (Berkeley, CA) | | |
| Sodium Citrate | Pacific Coast Chemicals, Co. | Dihydrate | 0.080 |
| | (Berkeley, CA) | | |
| Carrageenan | ET Horn/FMC Corporation | Seakem CM614 | 0.020 |
| | (Philadelphia, PA) | | |
| Sucralose | McNeil Nutritionals LLC | 25% Sol'n | 0.030 |
| | (Washington, PA) | | |
| Dipotassium | Pacific Coast Chemicals, Co. | Dibasic | 0.100 |
| Phosphate | (Berkeley, CA) | | |
| N&A French | Flavors of North America | 926.359 | 0.250 |
| Vanilla Flavor | (Carol Stream, IL) | | |
| N&A Vanilla | Ottens Flavors | 11295 | 0.100 |
| Flavor | (Philadelphia, PA) | | |
| Tricalcium | Pacific Coast Chemicals, Co. | None | 0.100 |
| Phosphate | (Berkeley, CA) | | |
| Magnesium | Pacific Coast Chemicals, Co. | Dibasic/Magnificent | 0.240 |
| Phosphate | (Berkeley, CA) | | |
| Citric | Pacific Coast Chemicals, Co. | Fine Granular | 0.120 |
| Acid | (Berkeley, CA) | | |
| Total | | | 100.000% |

In this example, a lightening mixer was employed in all of the mixing steps, and the pH of all liquid mixtures was adjusted as necessary to target a pH of about 6.6 to 7.0 by adding small quantities of either 1N citric acid or 1N sodium hydroxide thereto.

The water, Sucralose and maltitol syrup were mixed until a uniform liquid blend was produced, which took about 10 minutes, and then heated in a steam jacketed kettle to a temperature ranging between about 155° F. and 160° F. The potassium phosphate, magnesium phosphate, potassium citrate, sodium citrate, potassium chloride and citric acid were then mixed with the liquid blend until these ingredients were well incorporated into the blend, which took about 15 minutes.

Separately, the cellulose gum/gel, carrageenan, gum arabic, fructooligosaccharide and Fibersol 2 maltodextrin were dry blended with the M-100 maltodextrin until a uniform blend of these ingredients was produced, which took from about 20 to 30 minutes. The resulting dry blend was then mixed with the above liquid blend until the two blends were uniformly mixed together, which took about 30 minutes.

The crystalline fructose and the proteins (sodium caseinate, calcium sodium caseinate and soy protein isolate) were dry blended together until these ingredients were uniformly mixed together, which took from about 20 to 30 minutes, and then slowly added to, and mixed with, the above mixture, until these ingredients were well dispersed within the mixture, which took about 30 minutes. The oils (high oleic sunflower oil and high oleic canola oil), DHA and soy lecithin were then mixed into the blend, followed by the flavorings (N&A French vanilla flavor and N&A vanilla flavor), vitamin/mineral mix and tricalcium phosphate, until a uniform blend of these materials was produced, which took about 5 minutes. The pH of the resulting blend was adjusted, and the blend was reheated to a temperature of about 155° F., and then homogenized (using a conventional homogenizer) at a pressure of about 2,000+500 psi d/s.

The mixture was then filled using a standard filling apparatus into several 8-fluid ounce retort stable containers, and a retort process was performed, with a target of an F⊚ of about 9. (In a retort process, steam and pressure are applied, generally resulting in a temperature above about 100° C., and for a time generally determined by a process authority, but which is sufficient to kill spore-forming organisms of public health significance.) The pH of the mixture was recorded both prior to, and after, the retort process.

The resulting mixture was cooled to a temperature of less than about 100° F. and subsequently refrigerated. (Alternatively, the product may simply be allowed to cool to room temperature, for example, using a cooling bath, and may be labeled. Either before or after reaching room temperature.)

While the present invention has been described herein with specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the invention be limited only by the scope of the claims which follow, and that such claims be interpreted as broadly as is reasonable.

It is contemplated that nutritional supplements within the invention can also be used for animals, for example, as an additive for feeds. It is recommended that such a use of the nutritional supplements of the invention be used under the guidance and direction of a veterinarian.

Throughout this document, various books, patents, journal articles, web sites, federal regulations and other publications have been cited. The entireties of each of these books, patents, journal articles, web sites, federal regulations and other publications are hereby incorporated by reference herein.

1. A ready-to-drink liquid nutritional supplement for an administration to a human comprising:
(a) DHA, in an amount that is safe for consumption by humans, and is safe for developing fetuses or breast-fed babies of pregnant or lactating women, and that is effective for providing, maintaining or enhancing a health of a human's eyes, cardiovascular system, renal system, immunological system or neurological system, wherein the DHA is obtained from a source that is not an aquatic animal, and wherein the DHA has a high quality;
(b) folic acid, in an amount that is safe for consumption by humans, and is safe for developing fetuses or breast-fed babies of pregnant or lactating women, and that is effective for maintaining, or enhancing, cardiovascular function or neurological function in humans, preventing, or reducing the severity of, neural tube defects of fetuses that are developing in pregnant women, enhancing a break down of homocysteine in the body or providing a benefit related to insulin sensitivity;
(c) lutein, in an amount that is safe for consumption by humans, and is safe for developing fetuses or breast-fed babies of pregnant or lactating women, and that is effective for maintaining, or enhancing, eye, skin or cardiovascular health;
(d) L-arginine, in an amount that is safe for consumption by humans, and is safe for developing fetuses or breast-fed babies of pregnant or lactating women, and that is effective for enhancing vascular, neurological or immune function in humans or insulin sensitivity in diabetic humans;
(e) L-carnitine, in an amount that is safe for consumption by humans, and is safe for developing fetuses or breast-fed babies of pregnant or lactating women, and that is effective for enhancing a transport of one or more fatty acids into mitochondria present in cells of the human, a metabolism of one or more edible fats or oils that are present in the nutritional supplements or cardio-pulmonary function in the human;
(f) optionally, in addition to folic acid, one or more vitamins, minerals or vitamins and minerals in an amount that is safe for consumption by humans, and is safe for developing fetuses or breast-fed babies of pregnant or lactating women, and that is effective for providing a nutritional, medical or other health benefit to the human or for enhancing the general nutrition of the human;
(g) in addition to L-arginine and L-carnitine, one or more proteins or amino acids, or proteins and amino acids, in a combined amount that is safe for consumption by humans, and is safe for developing fetuses or breast-fed babies of pregnant or lactating women, and is effective for providing the human with from about 5% to about 50% of daily energy requirements;
(h) one or more carbohydrates in a combined amount that is safe for consumption by humans, and is safe for developing fetuses or breast-fed babies of pregnant or lactating women, and that is effective for providing the human with from about 25% to about 80% of daily energy requirements;
(i) in addition to DHA, one or more fats or oils, or fats and oils, in a combined amount that is safe for consumption by humans, and is safe for developing fetuses or breast-fed babies of pregnant or lactating women, and that is effective for providing a human with from about 10% to about 50% of the human's daily energy requirements, wherein the nutritional supplements contain an amount of trans fat that provides about 10% or less of daily energy requirements, an amount of saturated fat that provides about 10% or less of daily energy requirements, an amount of monounsaturated fats that provides from about 1% to about 20% of daily energy requirements, and an amount of polyunsaturated fats that provides from about 1% to about 10% of daily energy requirements;
(j) in addition to lutein, one or more antioxidant agents in a combined amount that is safe for consumption by humans, and that is safe for developing fetuses or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, or enhancing a stability or shelf life of the nutritional supplements, or of one or more ingredients included therein;
(k) one or more mouthfeel agents in a combined amount that is safe for consumption by humans, and that is safe for developing fetuses or breast-fed babies of pregnant or lactating women, and that is effective for providing, or enhancing, a stabilization of the nutritional supplements during thermal or other processes, providing a desirable mouthfeel, texture or thickness to the nutritional supplements or enhancing a mouthfeel, texture or thickness of the nutritional supplements;
(l) one or more emulsifiers in a combined amount that is safe for consumption by humans, and is safe for developing fetuses or breast-fed babies of pregnant or lactating women, and that is effective for forming, or aiding in the formation of, an emulsion, suspending solids within the nutritional supplements or emulsifying lipids within the nutritional supplements;
(m) water, in an amount that is sufficient to raise the total weight of the nutritional supplement to 100 percent;
wherein the nutritional supplement is a ready-to-drink liquid that can be consumed by humans without modification,
wherein the nutritional supplement does not contain a detectable amount of any ingredient that is obtained from an aquatic animal;
wherein the nutritional supplement does not contain a detectable amount of eicosapentaenoic acid;
wherein the nutritional supplement is effective for maintaining, or aiding in the maintenance of, a normal blood glucose level, or returning, or aiding in the returning of, an abnormal blood glucose level to a normal blood glucose level, in humans that are borderline diabetic, diabetic or otherwise glucose intolerant; and
wherein the nutritional supplement is safe for consumption by humans, including pregnant women, lactating women and women having childbearing potential that are attempting to become pregnant, and are safe for their developing fetuses and breast-fed babies.

2. The nutritional supplement of claim 1, wherein the nutritional supplement does not include any amount of any ingredient that is obtained from an aquatic animal.

3. The nutritional supplement of claim 1, wherein the nutritional supplement does not contain any eicosapentaenoic acid.

4. The nutritional supplement of claim 1, wherein the DHA is obtained from a plant, vegetable or fungal source.

5. The nutritional supplement of claim 4, wherein the DHA is obtained or derived from algae or microalgae.

6. The nutritional supplement of claim 1, wherein the DHA is present in an amount that ranges from about 20 mg to about 800 mg per day.

7. The nutritional supplement of claim 6, wherein the DHA is present in an amount that ranges from about 20 mg to about 200 mg per day.

8. The nutritional supplement of claim 1, wherein one 8-fluid ounce serving of the nutritional supplement contains from about 100 mg to about 200 mg of DHA.

9. The nutritional supplement of claim 1, wherein the lutein is present in an amount that ranges from about 0.2 mg to about 2.0 mg per day.

10. The nutritional supplement of claim 9, wherein the lutein is present in an amount that ranges from about 0.5 mg to about 1.0 mg per day.

11. The nutritional supplement of claim 1, wherein one 8-fluid ounce serving of the nutritional supplement contains about 0.5 mg of lutein.

12. The nutritional supplement of claim 1, wherein the one or more antioxidants are present in a combined amount that ranges from about 0.01 to about 3.0 weight percent of the total weight of the nutritional supplement.

13. The nutritional supplement of claim 1, wherein the one or more fats or oils include one or more omega-3 fatty acids, omega-6 fatty acids, omega-9 fatty acids or essential fatty acids, or a combination thereof.

14. The nutritional supplement of claim 1, wherein the nutritional supplement includes an amount of trans fat that provides about 10% or less of daily energy requirements, an amount of saturated fat that provides about 10% or less of daily energy requirements, an amount of monounsaturated fat that provides from about 1% to about 20% of daily energy requirements, and an amount of polyunsaturated fat that provides from about 1% to about 10% of daily energy requirements.

15. The nutritional supplement of claim 14, wherein the nutritional supplement includes an amount of saturated fat that provides about 7% or less of daily energy requirements.

16. The nutritional supplement of claim 1, wherein the one or more fats, oils or combination of fats and oils are present in a fat blend that comprises high oleic canola oil with either high oleic sunflower oil or high oleic safflower oil.

17. The nutritional supplement of claim 16, wherein the fat blend contains no trans fatty acids, no cholesterol and an amount of saturated fat that is about 3% of DV or less.

18. The nutritional supplement of claim 1, wherein the one or more fats, oils or combination of fats and oils are present in a combined amount that is effective for providing a human with from about 20% to about 35% of the human's daily energy requirements.

19. The nutritional supplement of claim 1, wherein the one or more fats, oils or combination of fats and oils are present in a combined amount that ranges from about 5 g to about 65 g.

20. The nutritional supplement of claim 19, wherein the one or more fats, oils or combination of fats and oils are present in a combined amount that ranges from about 5 g to about 30 g.

21. The nutritional supplement of claim 20, wherein the one or more fats, oils or combination of fats and oils are present in a combined amount that ranges from about 5 g to about 10 g.

22. The nutritional supplement of claim 21, wherein the one or more fats, oils or combination of fats and oils are present in a combined amount of about 8 grams.

23. The nutritional supplement of claim 1, wherein the one or more fats, oils or combination of fats and oils are present in a fat blend that includes:
(a) saturated fat in an amount ranging from about 5 to about 14 weight percent of the total fat blend;
(b) monounsaturated fat in an amount ranging from about 30 to about 80 weight percent of the total fat blend; and
(c) polyunsaturated fat in an amount ranging from about 10 to about 40 weight percent of the total fat blend.

24. The nutritional supplement of claim 23, wherein the fat blend includes:
(a) saturated fat in an amount ranging from about 5 to about 10 weight percent;
(b) monounsaturated fat in an amount ranging from about 50 to about 70 weight percent; and
(c) polyunsaturated fat in an amount ranging from about 15 to about 25 weight percent.

25. The nutritional supplement of claim 24, wherein the fat blend includes about 9 weight percent saturated fat, about 69 weight percent monounsaturated fat and about 22 weight percent polyunsaturated fat.

26. The nutritional supplement of claim 1, wherein the one or more fats, oils or combination of fats and oils are present in a combined amount that ranges from about 2.0 to about 7.0 weight percent of the total weight of the nutritional supplement.

27. The nutritional supplement of claim 26, wherein the one or more fats, oils or combination of fats and oils are present in a combined amount that ranges from about 3.0 to about 5.0 weight percent of the total weight of the nutritional supplement.

28. The nutritional supplement of claim 27, wherein the one or more fats, oils or combination of fats and oils are present in a combined amount that is about 4 weight percent of the total weight of the nutritional supplement.

29. The nutritional supplement of claim 1, wherein one 8-fluid ounce serving of the nutritional supplement contains a combined amount of about 8 grams of one or more edible fats, oils or combination of fats and oils, with about 0.5 g being saturated fats or oils, and with no trans fats or oils.

30. The nutritional supplement of claim 1, wherein the nutritional supplement includes one or more slow-digesting carbohydrates.

31. The nutritional supplement of claim 30, wherein the one or more slow-digesting carbohydrates are fructooligosaccharides, maltodextrin or fructose.

32. The nutritional supplement of claim 1, wherein the nutritional supplement includes a blend of two or more carbohydrates.

33. The nutritional supplement of claim 32, wherein at least one of the carbohydrates in the blend is a slow-digesting carbohydrate.

34. The nutritional supplement of claim 33, wherein the slow-digesting carbohydrate is fructooligosaccharides, maltodextrin or fructose.

35. The nutritional supplement of claim 32, wherein the blend of two or more carbohydrates is present in an amount that ranges from about 5 to about 30 weight percent of the total weight of the nutritional supplement.

36. The nutritional supplement of claim 35, wherein the blend of two or more carbohydrates is present in an amount that ranges from about 8 to about 16 weight percent of the total weight of the nutritional supplement.

37. The nutritional supplement of claim 36, wherein the blend of two or more carbohydrates is present in an amount that is about 12 weight percent of the total weight of the nutritional supplement.

38. The nutritional supplement of claim 32, wherein the blend of two or more carbohydrates includes:
(a) maltodextrin (10DE), in an amount ranging from about 40 to about 60 weight percent of the total carbohydrate blend;
(b) crystalline fructose, in an amount ranging from about 15 to about 35 weight percent of the total carbohydrate blend;
(c) maltitol, in an amount ranging from about 5 to about 30 weight percent of the total carbohydrate blend;
(d) maltodextrin (Fibersol 2), in an amount ranging from about 0.25 to about 6 weight percent of the total carbohydrate blend;
(e) gum arabic, in an amount ranging from about 0.25 to about 6 weight percent of the total carbohydrate blend; and
(f) cellulose gel, in an amount ranging from about 8 to about 35 weight percent of the total carbohydrate blend.

39. The nutritional supplement of claim 38, wherein the blend of two or more carbohydrates includes:
(a) maltodextrin (10DE), in an amount ranging from about 45 to about 55 weight percent of the carbohydrate blend;
(b) crystalline fructose, in an amount ranging from about 18 to about 28 weight percent of the carbohydrate blend;
(c) maltitol, in an amount ranging from about 10 to about 25 weight percent of the carbohydrate blend;
(d) maltodextrin (Fibersol 2), in an amount ranging from about 1 to about 5 weight percent of the carbohydrate blend;
(e) gum arabic, in an amount ranging from about 0.75 to about 3.0 weight percent of the carbohydrate blend; and
cellulose gel, in an amount ranging from about 13 to about 26 weight percent of the carbohydrate blend.

40. The nutritional supplement of claim 39, wherein the blend of two or more carbohydrates includes:
(a) about 49 weight percent of maltodextrin (10DE);
(b) about 23 weight percent of crystalline fructose;
(c) about 20 weight percent of maltitol;
(d) about 3 weight percent maltodextrin (Fibersol 2);
(e) about 2 weight percent gum arabic; and
(f) about 23 weight percent cellulose gel.

41. The nutritional supplement of claim 1, wherein the one or more carbohydrates is present in an amount that is effective for providing a human with from about 45% to about 65% of the human's daily energy requirements.

42. The nutritional supplement of claim 1, wherein the one or more carbohydrates is present in an amount that ranges from about 4 to about 12 weight percent of the nutritional supplement.

43. The nutritional supplement of claim 42, wherein the one or more carbohydrates is present in an amount that ranges from about 6 to about 9 weight percent of the nutritional supplement.

44. The nutritional supplement of claim 1, wherein the nutritional supplement contains one or more sugars in a combined amount that does not exceed about 35 weight percent of the total amount of carbohydrates that are present in the nutritional supplement.

45. The nutritional supplement of claim 44, wherein the combined amount of the one or more sugars does not exceed about 25 weight percent of the total amount of carbohydrates that are present in the nutritional supplement.

46. The nutritional supplement of claim 1, wherein one 8-ounce fluid serving of the nutritional supplement contains from about 3 g to about 15 g of sugar alcohols.

47. The nutritional supplement of claim 46, wherein one 8-ounce fluid serving of the nutritional supplement contains from about 4 g to about 7 g of sugar alcohols.

48. The nutritional supplement of claim 1, wherein one 8-ounce fluid serving of the nutritional supplement contains a combined amount of about 29 g of the one or more carbohydrates, about 6 g of which are sugar alcohols.

49. The nutritional supplement of claim 1, wherein one 8-ounce fluid serving of the nutritional supplement contains a combined amount of about 29 g of the one or more carbohydrates, about 5 g of which are sugars, and about 4 g of which are sugar alcohols.

50. The nutritional supplement of claim 1, wherein the nutritional supplement includes two or more additional proteins, and wherein the two or more additional proteins are a combination of milk protein and vegetable protein.

51. The nutritional supplement of claim 50, wherein the nutritional supplement includes two or more additional proteins, and wherein the two or more additional proteins include soy protein isolate.

52. The nutritional supplement of claim 52, wherein the two or more additional proteins are present in a combination that includes soy protein isolate with sodium caseinate or sodium calcium caseinate.

53. The nutritional supplement of claim 1, wherein the one or more additional proteins, amino acids, or proteins and amino acids are effective for providing the human with from about 10% to about 35% of the human's daily energy requirements.

54. The nutritional supplement of claim 53, wherein the one or more additional proteins, amino acids or proteins and amino acids are effective for providing the human with from about 17% to about 20% of the human's daily energy requirements.

55. The nutritional supplement of claim 1, wherein the one or more additional proteins, amino acids or proteins and amino acids are present in a combined amount that ranges from about 2 to about 10 weight percent of the nutritional supplement.

56. The nutritional supplement of claim 56, wherein the one or more additional proteins, amino acids or proteins and amino acids are present in a combined amount that ranges from about 3 to about 7 weight percent of the nutritional supplement.

57. The nutritional supplement of claim 1, wherein one 8-ounce fluid serving of the nutritional supplement contains, in addition to the amounts of L-arginine and L-carnitine, a combined amount of about 4.26 weight percent of proteins, amino acids, or proteins and amino acids.

58. The nutritional supplement of claim 1, wherein the nutritional supplement contains a blend of the one or more proteins, amino acids, or proteins and amino acids that includes sodium caseinate in an amount ranging from about 1.3 to about 2.5 weight percent of the total blend, calcium sodium caseinate in an amount ranging from about 1.3 to about 2.5 weight percent of the total blend, soy protein isolate in an amount ranging from about 0.6 to about 2.5 weight percent of the total blend and one or more amino acids in an amount ranging from about 0.001 to about 2.0 weight percent of the total blend.

59. The nutritional supplement of claim 1 wherein the nutritional supplement has a viscosity that ranges from about 20 cp to about 45 cp.

60. The nutritional supplement of claim 59 wherein the nutritional supplement has a viscosity that ranges from about 30 cp to about 44 cp.

61. The nutritional supplement of claim 1, wherein one eight-fluid ounce serving of the nutritional supplement contains a number of calories ranging from about 100 to about 400 calories, and wherein about 70 of the calories are derived from fats, oils or a combination of fats and oils.

62. The nutritional supplement of claim 1, wherein a percent of the total caloric value obtained from fats, oils or fats and oils ranges from about 15% to about 45%.

63. The nutritional supplement of claim 62, wherein a percent of the total caloric value obtained from fats, oils or fats and oils ranges from about 28% to about 35%.

64. The nutritional supplement of claim 63, wherein a percent of the total caloric value obtained from fats, oils or fats and oils is about 32%.

65. The nutritional supplement of claim 1, wherein:
(a) about 10% or fewer of total calories are derived from saturated fat;
(b) from about 5% to about 10% of total calories are derived from n-6 polyunsaturated fatty acids;
(c) from about 15% to about 25% of total calories are derived from monounsaturated fat; and
(d) about 3% or fewer of total calories are derived from trans fatty acids.

66. The nutritional supplement of claim 1, wherein:
(a) about 3% or fewer of total calories are derived from saturated fat;
(b) about 7% of total calories are derived from n-6 polyunsaturated fatty acids;
(c) about 23% of total calories are derived from monounsaturated fat; and
(d) about 0.5% or fewer of total calories are derived from trans fatty acids.

67. The nutritional supplement of claim 1, wherein a percent of total caloric value of the nutritional supplement that is obtained from the one or more carbohydrates ranges from about 30% to about 65%.

68. The nutritional supplement of claim 67, wherein a percent of the total caloric value of the nutritional supplement that is obtained from the one or more carbohydrates ranges from about 45% to about 55%.

69. The nutritional supplement of claim 68, wherein a percent of the total caloric value of the nutritional supplement that is obtained from the one or more carbohydrates is about 51%.

70. The nutritional supplement of claim 1, wherein a percent of the total caloric value of the nutritional supplement that is obtained from the one or more additional proteins, amino acids, or proteins and amino acids, ranges from about 7% to about 30%.

71. The nutritional supplement of claim 70, wherein a percent of the total caloric value of the nutritional supplement that is obtained from the one or more additional proteins, amino acids, or proteins and amino acids, ranges from about 13% to about 20%.

72. The nutritional supplement of claim 71, wherein a percent of the total caloric value of the nutritional supplement that is obtained from the one or more additional proteins, amino acids, or proteins and amino acids is about 17%.

73. The nutritional supplement of claim 1, wherein total calories of the nutritional supplement that are derived from a combination of proteins, amino acids and carbohydrates is less than about 80%.

74. The nutritional supplement of claim 73, wherein total calories of the nutritional supplement that are derived from a combination of proteins, amino acids and carbohydrates is less than about 75%.

75. The nutritional supplement of claim 74, wherein total calories of the nutritional supplement that are derived from a combination of proteins, amino acids and carbohydrates is less than about 70%.

76. The nutritional supplement of claim 1, wherein a percent of the total caloric value of the nutritional supplement obtained from the one or more carbohydrates is from about 45% to about 75% greater than a percent of the total caloric value of the nutritional supplement obtained from the one or more fats, oils or combination of fats and oils.

77. The nutritional supplement of claim 1, wherein a caloric distribution of the nutritional supplement comprises the one or more fats, oils or fats and oils in a combined amount ranging from about 20% to about 40%, the one or more carbohydrates in a combined amount ranging from about 40% to about 60% and the one or more proteins, amino acids or proteins and amino acids in a combined amount ranging from about 10% to about 25%.

78. The nutritional supplement of claim 1, wherein the one or more antioxidants are present in a combined amount of about 0.1 weight percent.

79. The nutritional supplement of claim 1, wherein the one or more antioxidants are ascorbyl palmitate, tocopherol, citric acid, ascorbic acid, vitamin A, vitamin C, vitamin E, beta-carotene, selenium or magnesium.

80. The nutritional supplement of claim 1, wherein the one or more mouthfeel agents comprise a mixture of gum arabic and carrageenan.

81. The nutritional supplement of claim 1, wherein the one or more mouthfeel agents are present in a combined amount ranging from about 0.2 to about 2.0 weight percent of a total weight of the nutritional supplement.

82. The nutritional supplement of claim 33, wherein the one or more mouthfeel agents are present in a combined amount ranging from about 0.5 to about 1.0 weight percent of a total weight of the nutritional supplement.

83. The nutritional supplement of claim 1, wherein the one or more emulsifiers are present in a combined amount ranging from about 0.2 to about 2.0 weight percent of a total weight of the nutritional supplement.

84. The nutritional supplement of claim 83, wherein the one or more emulsifiers are present in a combined amount ranging from about 0.5 to about 1.0 weight percent of a total weight of the nutritional supplement.

85. The nutritional supplement of claim 1, wherein the one or more emulsifiers are egg yolk, egg lecithin, soy lecithin, mono-glycerides or di-glycerides.

86. The nutritional supplement of claim 85, wherein the one or more emulsifiers include soy lecithin.

87. The nutritional supplement of claim 1, wherein the nutritional supplement is flavored with Sucralose in an amount that is effective for providing a pleasant taste to, or enhancing the taste of, the nutritional supplement.

88. The nutritional supplement of claim 1, wherein the nutritional supplement is in form of a drink or shake.

89. The nutritional supplement of claim 1, wherein the nutritional supplement may be orally consumed by a human.

90. The nutritional supplement of claim 1, wherein the nutritional supplement may be administered to a human enterally.

91. The nutritional supplement of claim 90, wherein the nutritional supplement may be administered to a human parenterally.

92. The nutritional supplement of claim 1, wherein the nutritional supplement has a caloric content ranging from about 100 to about 400 calories per serving.

93. The nutritional supplement of claim 92, wherein the nutritional supplement has a caloric content ranging from about 200 to about 250 calories per serving.

94. The nutritional supplement of claim 1 wherein the one or more carbohydrates are in a form of a carbohydrate blend, and wherein the carbohydrate blend includes about 49% maltodextrin (10DE), about 23% crystalline fructose, about 20% maltitol, about 3% maltodextrin (digestive resistant dextrin—Fibersol 2), about 2% gum arabic and about 3% cellulose gel.

95. The nutritional supplement of claim 1 wherein the one or more fats, oils or fats and oils is in a form of a fat blend, and wherein the fat blend includes about 9% of saturated fat, about 69% of monounsaturated fat and about 22% of polyunsaturated fat.

96. The nutritional supplement of claim 1 wherein a percent of the total caloric value obtained from the one or more proteins, amino acids or proteins and amino acids is about 17%, a percent of the total caloric value obtained from the one or more carbohydrates is about 51% and a percent of the total caloric value obtained from the one or more fats, oils or fats and oils is about 32%.

97. The nutritional supplement of claim 1 wherein a percent of total formula calories derived from saturated fat is about 3%, a percent of total formula calories derived from polyunsaturated fat is about 5%, a percent of total formula calories derived from trans fatty acids is less than about 0.5% and a percent of total formula calories derived from monounsaturated fatty acids is about 23%.

98. The nutritional supplement of claim 1, wherein the nutritional supplement includes from about 400 IU beta-carotene and about 800 IU vitamin A as palmitate to about 1000 IU beta-carotene and about 2000 vitamin A IU as palmitate per day.

99. The nutritional supplement of claim 98, wherein the nutritional supplement includes from about 600 IU beta-carotene and about 1000 IU vitamin A as palmitate to about 750 IU beta-carotene and about 1400 IU vitamin A as palmitate per day.

100. The nutritional supplement of claim 99, wherein the nutritional supplement includes about 700 IU beta-carotene and about 1200 IU vitamin A as palmitate per day.

101. The nutritional supplement of claim 1, wherein the folic acid is present in an amount ranging from about 0.2 mg to about 1.0 mg per day.

102. The nutritional supplement of claim 101, wherein the folic acid is present in an amount ranging from about 0.4 mg to about 0.8 mg per day.

103. The nutritional supplement of claim 1, wherein the nutritional supplement includes at least about 100% U.S. RDI for folic acid.

104. The nutritional supplement of claim 1, wherein the nutritional supplement contains from about 0% to about 75% of minimum U.S. RDI for iron.

105. The nutritional supplement of claim 104, wherein the nutritional supplement contains from about 15% to about 35% of minimum U.S. RDI for iron.

106. The nutritional supplement of claim 1, wherein the nutritional supplement additionally contains chromium in a daily quantity ranging from about 60 mcg to about 500 mcg.

107. The nutritional supplement of claim 1, wherein the nutritional supplement additionally contains chromium in an amount of at least about 100% of the U.S. RDI for chromium.

108. The nutritional supplement of claim 1, wherein the nutritional supplement additionally contains chromium, and wherein one 8-fluid ounce serving of the nutritional supplement contains an amount of chromium ranging from about 0.05 mg to about 0.20 mg.

109. The nutritional supplement of claim 108, wherein the one 8-fluid ounce serving contains about 0.12 mg of chromium.

110. The nutritional supplement of claim 1, wherein the nutritional supplement additionally contains selenium in an amount that is effective for enhancing a metabolism of fat in a human or having activity as an antioxidant.

111. The nutritional supplement of claim 110, wherein the selenium is in the form of sodium selenite, and the nutritional supplement does not include sodium selenate.

112. The nutritional supplement of claim 111, wherein the sodium selenite is present in an amount ranging from about 10 mcg to about 200 mcg per day.

113. The nutritional supplement of claim 112, wherein the sodium selenite is present in an amount ranging from about 20 mcg to about 70 mcg per day.

114. The nutritional supplement of claim 1, wherein the L-arginine is present in an amount ranging from about 20 mg to about 3 g per day.

115. The nutritional supplement of claim 114, wherein the L-arginine is present in an amount ranging from about 500 mg to about 1.5 g per day.

116. The nutritional supplement of claim 115, wherein the L-arginine is present in an amount of about 1 g per day.

117. The nutritional supplement of claim 1, wherein the L-carnitine is present in an amount ranging from about 10 mg to about 150 mg per day.

118. The nutritional supplement of claim 117, wherein the L-carnitine is present in an amount ranging from about 30 mg to about 90 mg per day.

119. The nutritional supplement of claim 1, wherein one 8-fluid ounce serving of the nutritional supplement contains about 31 mg of L-carnitine.

120. The nutritional supplement of claim 1, wherein the nutritional supplement additionally contains m-inositol in an amount ranging from about 20 mg to about 400 mg per day.

121. The nutritional supplement of claim 120, wherein the m-inositol is present in an amount ranging from about 100 mg to about 200 mg per day.

122. The nutritional supplement of claim 1, wherein one 8-fluid ounce serving of the nutritional supplement contains about 186 mg of m-inositol.

123. The nutritional supplement of claim 1, wherein one 8-fluid ounce serving of the nutritional supplement contains about 1.20 weight percent of fiber of a total weight of the nutritional supplement.

124. The nutritional supplement of claim 1, wherein the nutritional supplement has a total solids content ranging from about 70 to about 95 percent by weight.

125. The nutritional supplement of claim 1, wherein the nutritional supplement can serve as a sole source of nutrition for a human.

126. The nutritional supplement of claim 1, wherein the nutritional supplement cannot serve as a sole source of nutrition for a human.

127. The nutritional supplement of claim 1, wherein the nutritional supplement does not contain any detectable level of gellan gum.

128. The nutritional supplement of claim 1, wherein the nutritional supplement does not contain any detectable level of taurine.

129. The nutritional supplement of claim 1, wherein the nutritional supplement does not contain any detectable level of soy polysaccharide.

130. The nutritional supplement of claim 1, wherein the nutritional supplement does not include insulin.

131. The nutritional supplement of claim 1, wherein one 8-fluid ounce serving of the nutritional supplement includes the following ingredients, and amounts thereof:

| Ingredients | Formula % by Weight |
|---|---|
| Water | 78.060 |
| Maltodextrin | 5.870 |
| High Oleic Sunflower Oil | 2.440 |
| Crystalline Fructose | 2.420 |
| Maltitol Syrup | 2.810 |
| Sodium Caseinate | 1.690 |
| Calcium Sodium Caseinate | 1.690 |
| Soy Protein Isolate | 0.880 |
| Fructooligosaccharides | 0.440 |
| Vitamin/Mineral Mix | 0.790 |
| Fibersol2 - Maltodextrin | 0.320 |
| High Oleic Canola Oil | 0.330 |
| Gum Arabic | 0.240 |
| Cellulose Gum/Gel | 0.400 |
| Soy Lecithin | 0.170 |
| Potassium Chloride | 0.100 |
| Docosahexaenoic acid (DHA) | 0.230 |
| Potassium Citrate | 0.080 |
| Sodium Citrate | 0.080 |
| Carrageenan | 0.020 |
| Sucralose | 0.030 |
| Potassium Phosphate | 0.100 |
| Natural and Artificial French Vanilla Flavor | 0.250 |
| Natural and Artificial Vanilla Flavor | 0.100 |
| Tricalcium Phosphate | 0.100 |
| Magnesium Phosphate | 0.240 |
| Citric Acid | 0.120 |
| Total | 100% |

132. The nutritional supplement of claim 131, wherein the one 8-fluid ounce serving contains 220 calories, and wherein about 70 of the calories are provided by the one or more fats, oils or fats and oils.

133. The nutritional supplement of claim 132, wherein the one 8-fluid ounce serving contains the following weight percents of the one or more proteins, amino acids or proteins and amino acids, the one or more carbohydrates and the one or more fats, oils or fats and oils:
(a) about 4% of the one or more proteins, amino acids or proteins and amino acids;
(b) about 12% of the one or more carbohydrate; and
(c) about 4% of the one or more fats, oils or fats and oils.

134. The nutritional supplement of claim 133, wherein the one or more carbohydrates are present in a carbohydrate blend that includes:
(a) about 49% maltodextrin (10DE);
(b) about 23% crystalline fructose;
(c) about 20% maltitol;
(d) about 3% maltodextrin (digestive resistant dextrin—Fibersol 2);
(e) about 2% gum arabic; and
(f) about 3% cellulose gel.

135. The nutritional supplement of claim 134, wherein the one or more fats, oils or fats and oils are present in a fat blend that includes:
(a) about 9% of saturated fat;
(b) about 69% of monounsaturated fat; and
(c) about 22% of polyunsaturated fat.

136. The nutritional supplement of claim 135, wherein a percent of total caloric value obtained from the one or more proteins, amino acids or proteins and amino acids in the nutritional supplement is about 17%, a percent of total caloric value obtained from the carbohydrate blend is about 51%, a percent of total caloric value obtained from the fat blend is about 32%, total calories derived from saturated fat is about 3%, total calories derived from polyunsaturated fat is about 5%, a percent of the total calories derived from trans fatty acids is less than about 0.5%, a percent of the total calories derived from monounsaturated fatty acids is about 23%, and a dextrose equivalence of starch employed is 10.

137. The nutritional supplement of claim 135, wherein the nutritional supplement has a viscosity of about 38.2 cp.

138. The nutritional supplement of claim 136, wherein the nutritional supplement has a total solids content of about 21.94% by weight.

139. The nutritional supplement of claim 137, wherein the nutritional supplement includes about 4 weight percent of the one or more proteins, amino acids or proteins and amino acids, about 12 weight percent of the one or more carbohydrates and about 4 weight percent of the one or more fats, oils or fats and oils.

140. A liquid concentrate nutritional supplement for an administration to humans comprising a ready-to-drink liquid nutritional supplement of claim 1, wherein an amount of water has been removed from the ready-to-drink liquid nutritional supplement in a manner that is effective for forming a liquid concentrate nutritional supplement from the ready-to-drink liquid nutritional supplement, and wherein the liquid concentrate can be transformed into a ready-to-drink liquid nutritional supplement by an addition of an amount of water that is effective for transforming the liquid concentrate into a ready-to-drink liquid nutritional supplement.

141. A solid concentrate nutritional supplement for an administration to humans comprising a ready-to-drink liquid nutritional supplement of claim 1, wherein an amount of water has been removed from the ready-to-drink liquid nutritional supplement in a manner that is effective for forming a solid concentrate nutritional supplement from the ready-to-drink liquid nutritional supplement, and wherein the solid concentrate can be transformed into a ready-to-drink liquid nutritional supplement by an addition of an amount of water that is effective for transforming the solid concentrate into a ready-to-drink liquid nutritional supplement.

142. The nutritional supplement of claim 140, wherein the solid concentrate is a powder.

143. The nutritional supplement of claim 141, wherein the powder is a spray-dried powder.

144. The nutritional supplement of claim 141, wherein the solid concentrate is an agglomerated powder.

145. A nutritional supplement in the form of a food bar for an administration to a human comprising:
(a) DHA, in an amount that is safe for consumption by humans, and is safe for developing fetuses or breast-fed babies of pregnant or lactating women, and that is effective for providing, maintaining or enhancing a health of a human's eyes, cardiovascular system, renal system, immunological system or neurological system, wherein the DHA is obtained from a source that is not an aquatic animal, and wherein the DHA has a high quality;
(b) folic acid, in an amount that is safe for consumption by humans, and is safe for developing fetuses or breast-fed babies of pregnant or lactating women, and that is effective for maintaining, or enhancing, cardiovascular function or neurological function in humans, preventing, or reducing the severity of, neural tube defects of fetuses that are developing in pregnant women, enhancing a break down of homocysteine in the body or providing a benefit related to insulin sensitivity;
(c) lutein, in an amount that is safe for consumption by humans, and is safe for developing fetuses or breast-fed babies of pregnant or lactating women, and that is effective for maintaining, or enhancing, eye, skin or cardiovascular health;
(d) L-arginine, in an amount that is safe for consumption by humans, and is safe for developing fetuses or breast-fed babies of pregnant or lactating women, and that is effective for enhancing vascular, neurological or immune function in humans or insulin sensitivity in diabetic humans;
(e) L-carnitine, in an amount that is safe for consumption by humans, and is safe for developing fetuses or breast-fed babies of pregnant or lactating women, and that is effective for enhancing a transport of one or more fatty acids into mitochondria present in cells of the human, a metabolism of one or more edible fats or oils that are present in the nutritional supplements or cardio-pulmonary function in the human;
(f) optionally, in addition to folic acid, one or more vitamins, minerals or vitamins and minerals in an amount that is safe for consumption by humans, and is safe for developing fetuses or breast-fed babies of pregnant or lactating women, and that is effective for providing a nutritional, medical or other health benefit to the human or for enhancing the general nutrition of the human;
(g) in addition to L-arginine and L-carnitine, one or more proteins or amino acids, or proteins and amino acids, in a combined amount that is safe for consumption by humans, and is safe for developing fetuses or breast-fed babies of pregnant or lactating women, and is effective for providing the human with from about 5% to about 50% of daily energy requirements;
(h) one or more carbohydrates in a combined amount that is safe for consumption by humans, and is safe for developing fetuses or breast-fed babies of pregnant or lactating women, and that is effective for providing the human with from about 25% to about 80% of daily energy requirements;
(i) in addition to DHA, one or more fats or oils, or fats and oils, in a combined amount that is safe for consumption by humans, and is safe for developing fetuses or breast-fed babies of pregnant or lactating women, and that is effective for providing a human with from about 10% to about 50% of the human's daily energy requirements, wherein the nutritional supplements contain an amount of trans fat that provides about 10% or less of daily energy requirements, an amount of saturated fat that provides about 10% or less of daily energy requirements, an amount of monounsaturated fats that provides from about 1% to about 20% of daily energy requirements, and an amount of polyunsaturated fats that provides from about 1% to about 10% of daily energy requirements;
(j) in addition to lutein, one or more antioxidant agents in a combined amount that is safe for consumption by humans, and that is safe for developing fetuses or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, or enhancing a stability or shelf life of the nutritional supplements, or of one or more ingredients included therein;
(k) optionally, one or more mouthfeel agents in a combined amount that is safe for consumption by humans, and that is safe for developing fetuses or breast-fed babies of pregnant or lactating women, and that is effective for providing, or enhancing, a stabilization of the nutritional supplements during thermal or other processes, providing a desirable mouthfeel, texture or thickness to the nutritional supplements or enhancing a mouthfeel, texture or thickness of the nutritional supplements;
(l) one or more emulsifiers in a combined amount that is safe for consumption by humans, and is safe for developing fetuses or breast-fed babies of pregnant or lactating women, and that is effective for forming, or aiding in the formation of, an emulsion, suspending solids within the nutritional supplements or emulsifying lipids within the nutritional supplements;
(m) one or more fillers, in an amount that is sufficient to raise the total weight of the nutritional supplement to 100 percent;
wherein the nutritional supplement is a food bar that can be consumed by humans without modification,
wherein the nutritional supplement has a moisture content ranging from about 0 to about 10 weight percent of the total weight of the nutritional supplement;
wherein the nutritional supplement does not contain a detectable amount of any ingredient that is obtained from an aquatic animal;
wherein the nutritional supplement does not contain a detectable amount of eicosapentaenoic acid;
wherein the nutritional supplement is effective for maintaining, or aiding in the maintenance of, a normal blood glucose level, or returning, or aiding in the returning of, an abnormal blood glucose level to a normal blood glucose level, in humans that are borderline diabetic, diabetic or otherwise glucose intolerant; and
wherein the nutritional supplement is safe for consumption by humans, including pregnant women, lactating women and women having childbearing potential that are attempting to become pregnant, and are safe for their developing fetuses and breast-fed babies.

146. A method for providing one or more nutritional, medical or other health benefits to a human comprising administering to the human a nutritional supplement of claim 1 in an amount that is effective for providing one or more nutritional, medical and/or other health benefits to the human.

147. A method for providing one or more nutritional, medical or other health benefits to a human comprising administering to the human a nutritional supplement of claim 144 in an amount that is effective for providing one or more nutritional, medical and/or other health benefits to the human.

148. A method for maintaining, or aiding in the maintenance of, a normal blood glucose level of a diabetic, borderline diabetic or other human that has glucose intolerance comprising administering to the human a nutritional supplement of claim 1 in an amount that is effective for maintaining, or aiding in the maintenance of, the blood glucose level of the human at a normal level.

149. A method for maintaining, or aiding in the maintenance of, a normal blood glucose level of a diabetic, borderline diabetic or other human that has glucose intolerance comprising administering to the human a nutritional supplement of claim 144 in an amount that is effective for maintaining, or aiding in the maintenance of, the blood glucose level of the human at a normal level.

150. A method for returning, or aiding in the return of, an abnormal blood glucose level of a diabetic, borderline diabetic or other human that has glucose intolerance to a normal blood glucose level comprising administering to the human a nutritional supplement of claim 1 in an amount that is effective for returning, or aiding in the return of, the blood glucose level of the human to a normal level.

151. A method for returning, or aiding in the return of, an abnormal blood glucose level of a diabetic, borderline diabetic or other human that has glucose intolerance to a normal blood glucose level comprising administering to the human a nutritional supplement of claim 144 in an amount that is effective for returning, or aiding in the return of, the blood glucose level of the human to a normal level.

152. A method for preventing, or reducing the likelihood of, a pre-diabetic or borderline diabetic condition of a human from transforming into diabetes or another glucose intolerance disorder comprising administering to the human a nutritional supplement of claim 1 in an amount that is effective for preventing, or reducing the likelihood of, the human from having a pre-diabetic or borderline diabetic condition transforming into diabetes or another glucose intolerance disorder.

153. A method for preventing, or reducing the likelihood of, a pre-diabetic or borderline diabetic condition of a human from transforming into diabetes or another glucose intolerance disorder comprising administering to the human a nutritional supplement of claim 144 in an amount that is effective for preventing, or reducing the likelihood of, the human from having a pre-diabetic or borderline diabetic condition transforming into diabetes or another glucose intolerance disorder.

154. A method for preventing, or reducing the likelihood of, a diabetic, borderline diabetic or human that has glucose intolerance from experiencing one or more symptoms or complications related to, or produced by, diabetes or glucose intolerance comprising administering to the human a nutritional supplement of claim 1 in an amount that is effective for preventing, or reducing the likelihood of, the human from experiencing such symptoms or complications.

155. A method for preventing, or reducing the likelihood of, a diabetic, borderline diabetic or human that has glucose intolerance from experiencing one or more symptoms or complications related to, or produced by, diabetes or glucose intolerance comprising administering to the human a nutritional supplement of claim 144 in an amount that is effective for preventing, or reducing the likelihood of, the human from experiencing such symptoms or complications.

156. A method for preventing, or reducing the likelihood of, a human from having pre-mature cardiovascular disease comprising administering to the human a nutritional supplement of claim 1 in an amount that is effective for preventing, or reducing the likelihood of, a human from having pre-mature cardiovascular disease.

157. A method for preventing, or reducing the likelihood of, a human from having pre-mature cardiovascular disease comprising administering to the human a nutritional supplement of claim 144 in an amount that is effective for preventing, or reducing the likelihood of, a human from having pre-mature cardiovascular disease.

158. A ready-to-drink liquid nutritional supplement for an administration to a human comprising:
(a) one or more omega-3 fatty acids, in a combined amount that is safe for consumption by humans, and that is effective for providing, maintaining or enhancing a health of a human's eyes, cardiovascular system, renal system, immunological system or neurological system, or for providing one or more other nutritional, health or medical benefits to the human, wherein the one or more omega-3 fatty acids are obtained from, or present in, a source that does not contain a level of mercury that is detectable and harmful, a level of any other hazardous heavy metal that is detectable and harmful, a level of a pesticide that is detectable and harmful or a level of an industrial chemical that is detectable and harmful, and wherein the source contains at least about 10 weight percent of the one or more omega-3 fatty acids;
(b) folic acid, in an amount that is safe for consumption by humans, and is safe for developing fetuses or breast-fed babies of pregnant or lactating women, and that is effective for maintaining, or enhancing, cardiovascular function and/or neurological function in humans, preventing, or reducing the severity of, neural tube defects of fetuses that are developing in pregnant women, enhancing a break down of homocysteine in the body and/or providing a benefit related to insulin sensitivity;
(c) lutein, in an amount that is safe for consumption by humans, and is safe for developing fetuses or breast-fed babies of pregnant or lactating women, and that is effective for maintaining, or enhancing, eye, skin and/or cardiovascular health;
(d) L-arginine, in an amount that is safe for consumption by humans, and is safe for developing fetuses or breast-fed babies of pregnant or lactating women, and that is effective for enhancing vascular, neurological or immune function in humans or insulin sensitivity in diabetic humans;
(e) L-carnitine, in an amount that is safe for consumption by humans, and is safe for developing fetuses or breast-fed babies of pregnant or lactating women, and that is effective for enhancing a transport of one or more fatty acids into mitochondria present in cells of the human, a metabolism of one or more edible fats and/or oils that are present in the nutritional supplements and/or cardio-pulmonary function in the human;
(f) optionally, in addition to folic acid, one or more vitamins and/or minerals, in an amount that is safe for consumption by humans, and is safe for developing fetuses or breast-fed babies of pregnant or lactating women, and that is effective for providing a nutritional, medical and/or other health benefit to the human and/or for enhancing the general nutrition of the human;
(g) in addition to L-arginine and L-carnitine, one or more proteins and/or amino acids in a combined amount that is safe for consumption by humans, and is safe for developing fetuses or breast-fed babies of pregnant or lactating women, and is effective for providing the human with from about 5% to about 50% of daily energy requirements;
(h) one or more carbohydrates in a combined amount that is safe for consumption by humans, and is safe for developing fetuses or breast-fed babies of pregnant or lactating women, and that is effective for providing the human with from about 25% to about 80% of daily energy requirements;
(i) in addition to the one or more omega-3 fatty acids, one or more fats and/or oils in a combined amount that is safe for consumption by humans, and is safe for developing fetuses or breast-fed babies of pregnant or lactating women, and that is effective for providing a human with from about 10% to about 50% of the human's daily energy requirements, wherein the nutritional supplements contain an amount of trans fat that provides about 10% or less of daily energy requirements, an amount of saturated fat that provides about 10% or less of daily energy requirements, an amount of monounsaturated fats that provides from about 1% to about 20% of daily energy requirements, and an amount of polyunsaturated fats that provides from about 1% to about 10% of daily energy requirements;
(j) in addition to lutein, one or more antioxidant agents in a combined amount that is safe for consumption by humans, and that is safe for developing fetuses or breast-fed babies of pregnant or lactating women, and that is effective for preventing, or reducing the rate of, an oxidation of one or more ingredients included in the nutritional supplements, preventing, aiding in the prevention of, or reducing an amount of, oxygen-based damage to cells, and/or enhancing a stability and/or shelf life of the nutritional supplements, or of one or more ingredients included therein;
(k) one or more mouthfeel agents in a combined amount that is safe for consumption by humans, and that is safe for developing fetuses or breast-fed babies of pregnant or lactating women, and that is effective for providing, or enhancing, a stabilization of the nutritional supplements during thermal or other processes, providing a desirable mouthfeel, texture or thickness to the nutritional supplements, and/or enhancing a mouthfeel, texture or thickness of the nutritional supplements;
(l) one or more emulsifiers in a combined amount that is safe for consumption by humans, and is safe for developing fetuses or breast-fed babies of pregnant or lactating women, and that is effective for forming, or aiding in the formation of, an emulsion, suspending solids within the nutritional supplements and/or emulsifying lipids within the nutritional supplements; and
(m) water, in an amount that is sufficient to raise the total weight of the nutritional supplement to 100 percent;
wherein the nutritional supplement is a ready-to-drink liquid that can be consumed by humans without modification,
wherein the nutritional supplement is effective for maintaining, or aiding in the maintenance of, a normal blood glucose level, or returning, or aiding in the returning of, an abnormal blood glucose level to a normal blood glucose level, in humans that are borderline diabetic, diabetic or otherwise glucose intolerant; and
wherein the nutritional supplement is safe for consumption by humans, and is safe for breast-fed babies of lactating women.

159. The nutritional supplement of claim 158 wherein the one or more omega-3 fatty acids are DHA, docosapentaenoic acid, ALA, EPA, eicosatetraenoic acid, moroctic acid, heneicosapentenoic acid, linoleic acid, alpha-linolenic acid or a combination thereof.

160. The nutritional supplement of claim 158, wherein the nutritional supplement does not include a detectable amount of any ingredient that is obtained from an aquatic animal.

161. The nutritional supplement of claim 158, wherein the nutritional supplement does not contain a detectable level of eicosapentaenoic acid.

162. The nutritional supplement of claim 158, wherein the nutritional supplement contains DHA, and wherein the DHA is obtained from a plant, vegetable or fungal source.

163. The nutritional supplement of claim 162, wherein the DHA is obtained or derived from algae or microalgae.

164. The nutritional supplement of claim 158, wherein the DHA is present in an amount that ranges from about 20 mg to about 800 mg per day.

165. The nutritional supplement of claim 164, wherein the DHA is present in an amount that ranges from about 20 mg to about 200 mg per day.

166. The nutritional supplement of claim 158, wherein one 8-fluid ounce serving of the nutritional supplement contains from about 100 mg to about 200 mg of DHA.

167. The nutritional supplement of claim 158, wherein the lutein is present in an amount that ranges from about 0.2 mg to about 2.0 mg per day.

168. The nutritional supplement of claim 167, wherein the lutein is present in an amount that ranges from about 0.5 mg to about 1.0 mg per day.

169. The nutritional supplement of claim 158, wherein one 8-fluid ounce serving of the nutritional supplement contains about 0.5 mg of lutein.

170. The nutritional supplement of claim 158, wherein the one or more antioxidants are present in a combined amount that ranges from about 0.01 to about 3.0 weight percent of the total weight of the nutritional supplement.

171. The nutritional supplement of claim 158, wherein the one or more fats or oils include one or more omega-6 fatty acids, omega-9 fatty acids or essential fatty acids, or a combination thereof.

172. The nutritional supplement of claim 158, wherein the nutritional supplement includes an amount of trans fat that provides about 10% or less of daily energy requirements, an amount of saturated fat that provides about 10% or less of daily energy requirements, an amount of monounsaturated fat that provides from about 1% to about 20% of daily energy requirements, and an amount of polyunsaturated fat that provides from about 1% to about 10% of daily energy requirements.

173. The nutritional supplement of claim 172, wherein the nutritional supplement includes an amount of saturated fat that provides about 7% or less of daily energy requirements.

174. The nutritional supplement of claim 158, wherein the one or more fats, oils or combination of fats and oils are present in a fat blend that comprises high oleic canola oil with either high oleic sunflower oil or high oleic safflower oil.

175. The nutritional supplement of claim 174, wherein the fat blend contains no trans fatty acids, no cholesterol and an amount of saturated fat that is about 3% of DV or less.

176. The nutritional supplement of claim 158, wherein the one or more fats, oils or combination of fats and oils are present in a combined amount that is effective for providing a human with from about 20% to about 35% of the human's daily energy requirements.

177. The nutritional supplement of claim 158, wherein the one or more fats, oils or combination of fats and oils are present in a combined amount that ranges from about 5 g to about 65 g.

178. The nutritional supplement of claim 177, wherein the one or more fats, oils or combination of fats and oils are present in a combined amount that ranges from about 5 g to about 30 g.

179. The nutritional supplement of claim 178, wherein the one or more fats, oils or combination of fats and oils are present in a combined amount that ranges from about 5 g to about 10 g.

180. The nutritional supplement of claim 179, wherein the one or more fats, oils or combination of fats and oils are present in a combined amount of about 8 grams.

181. The nutritional supplement of claim 158, wherein the one or more fats, oils or combination of fats and oils are present in a fat blend that includes:
(a) saturated fat in an amount ranging from about 5 to about 14 weight percent of the total fat blend;
(b) monounsaturated fat in an amount ranging from about 30 to about 80 weight percent of the total fat blend; and
(c) polyunsaturated fat in an amount ranging from about 10 to about 40 weight percent of the total fat blend.

182. The nutritional supplement of claim 181, wherein the fat blend includes:
(a) saturated fat in an amount ranging from about 5 to about 10 weight percent;
(b) monounsaturated fat in an amount ranging from about 50 to about 70 weight percent; and
(c) polyunsaturated fat in an amount ranging from about 15 to about 25 weight percent.

183. The nutritional supplement of claim 182, wherein the fat blend includes about 9 weight percent saturated fat, about 69 weight percent monounsaturated fat and about 22 weight percent polyunsaturated fat.

184. The nutritional supplement of claim 158, wherein the one or more fats, oils or combination of fats and oils are present in a combined amount that ranges from about 2.0 to about 7.0 weight percent of the total weight of the nutritional supplement.

185. The nutritional supplement of claim 184, wherein the one or more fats, oils or combination of fats and oils are present in a combined amount that ranges from about 3.0 to about 5.0 weight percent of the total weight of the nutritional supplement.

186. The nutritional supplement of claim 186, wherein the one or more fats, oils or combination of fats and oils are present in a combined amount that is about 4 weight percent of the total weight of the nutritional supplement.

187. The nutritional supplement of claim 158, wherein one 8-fluid ounce serving of the nutritional supplement contains a combined amount of about 8 grams of one or more edible fats, oils or combination of fats and oils, with about 0.5 g being saturated fats or oils, and with no trans fats or oils.

188. The nutritional supplement of claim 158, wherein the nutritional supplement includes one or more slow-digesting carbohydrates.

189. The nutritional supplement of claim 188, wherein the one or more slow-digesting carbohydrates are fructooligosaccharides, maltodextrin or fructose.

190. The nutritional supplement of claim 158, wherein the nutritional supplement includes a blend of two or more carbohydrates.

191. The nutritional supplement of claim 190, wherein at least one of the carbohydrates in the blend is a slow-digesting carbohydrate.

192. The nutritional supplement of claim 191, wherein the slow-digesting carbohydrate is fructooligosaccharides, maltodextrin or fructose.

193. The nutritional supplement of claim 190, wherein the blend of two or more carbohydrates is present in an amount that ranges from about 5 to about 30 weight percent of the total weight of the nutritional supplement.

194. The nutritional supplement of claim 193, wherein the blend of two or more carbohydrates is present in an amount that ranges from about 8 to about 16 weight percent of the total weight of the nutritional supplement.

195. The nutritional supplement of claim 194, wherein the blend of two or more carbohydrates is present in an amount that is about 12 weight percent of the total weight of the nutritional supplement.

196. The nutritional supplement of claim 190, wherein the blend of two or more carbohydrates includes:
(a) maltodextrin (10DE), in an amount ranging from about 40 to about 60 weight percent of the total carbohydrate blend;
(b) crystalline fructose, in an amount ranging from about 15 to about 35 weight percent of the total carbohydrate blend;
(c) maltitol, in an amount ranging from about 5 to about 30 weight percent of the total carbohydrate blend;
(d) maltodextrin (Fibersol 2), in an amount ranging from about 0.25 to about 6 weight percent of the total carbohydrate blend;
(e) gum arabic, in an amount ranging from about 0.25 to about 6 weight percent of the total carbohydrate blend; and cellulose gel, in an amount ranging from about 8 to about 35 weight percent of the total carbohydrate blend.

197. The nutritional supplement of claim 196, wherein the blend of two or more carbohydrates includes:
(a) maltodextrin (10DE), in an amount ranging from about 45 to about 55 weight percent of the carbohydrate blend;
(b) crystalline fructose, in an amount ranging from about 18 to about 28 weight percent of the carbohydrate blend;
(c) maltitol, in an amount ranging from about 10 to about 25 weight percent of the carbohydrate blend;
(d) maltodextrin (Fibersol 2), in an amount ranging from about 1 to about 5 weight percent of the carbohydrate blend;
(e) gum arabic, in an amount ranging from about 0.75 to about 3.0 weight percent of the carbohydrate blend; and
(f) cellulose gel, in an amount ranging from about 13 to about 26 weight percent of the carbohydrate blend.

198. The nutritional supplement of claim 197, wherein the blend of two or more carbohydrates includes:
(a) about 49 weight percent of maltodextrin (10DE);
(b) about 23 weight percent of crystalline fructose;
(c) about 20 weight percent of maltitol;
(d) about 3 weight percent maltodextrin (Fibersol 2);
(e) about 2 weight percent gum arabic; and
(f) about 23 weight percent cellulose gel.

199. The nutritional supplement of claim 158, wherein the one or more carbohydrates is present in an amount that is effective for providing a human with from about 45% to about 65% of the human's daily energy requirements.

200. The nutritional supplement of claim 158, wherein the one or more carbohydrates is present in an amount that ranges from about 4 to about 12 weight percent of the nutritional supplement.

201. The nutritional supplement of claim 200, wherein the one or more carbohydrates is present in an amount that ranges from about 6 to about 9 weight percent of the nutritional supplement.

202. The nutritional supplement of claim 158, wherein the nutritional supplement contains one or more sugars in a combined amount that does not exceed about 35 weight percent of the total amount of carbohydrates that are present in the nutritional supplement.

203. The nutritional supplement of claim 202, wherein the combined amount of the one or more sugars does not exceed about 25 weight percent of the total amount of carbohydrates that are present in the nutritional supplement.

204. The nutritional supplement of claim 158, wherein one 8-ounce fluid serving of the nutritional supplement contains from about 3 g to about 15 g of sugar alcohols.

205. The nutritional supplement of claim 204, wherein one 8-ounce fluid serving of the nutritional supplement contains from about 4 g to about 7 g of sugar alcohols.

206. The nutritional supplement of claim 158, wherein one 8-ounce fluid serving of the nutritional supplement contains a combined amount of about 29 g of the one or more carbohydrates, about 6 g of which are sugar alcohols.

207. The nutritional supplement of claim 158, wherein one 8-ounce fluid serving of the nutritional supplement contains a combined amount of about 29 g of the one or more carbohydrates, about 5 g of which are sugars, and about 4 g of which are sugar alcohols.

208. The nutritional supplement of claim 158, wherein the nutritional supplement includes two or more additional proteins, and wherein the two or more additional proteins are a combination of milk protein and vegetable protein.

209. The nutritional supplement of claim 158, wherein the nutritional supplement includes two or more additional proteins, and wherein the two or more additional proteins include soy protein isolate.

210. The nutritional supplement of claim 209, wherein the two or more additional proteins are present in a combination that includes soy protein isolate with sodium caseinate or sodium calcium caseinate.

211. The nutritional supplement of claim 158, wherein the one or more additional proteins, amino acids, or proteins and amino acids are effective for providing the human with from about 10% to about 35% of the human's daily energy requirements.

212. The nutritional supplement of claim 211, wherein the one or more additional proteins, amino acids or proteins and amino acids are effective for providing the human with from about 17% to about 20% of the human's daily energy requirements.

213. The nutritional supplement of claim 158, wherein the one or more additional proteins, amino acids or proteins and amino acids are present in a combined amount that ranges from about 2 to about 10 weight percent of the nutritional supplement.

214. The nutritional supplement of claim 213, wherein the one or more additional proteins, amino acids or proteins and amino acids are present in a combined amount that ranges from about 3 to about 7 weight percent of the nutritional supplement.

215. The nutritional supplement of claim 158, wherein one 8-ounce fluid serving of the nutritional supplement contains, in addition to the amounts of L-arginine and L-carnitine, a combined amount of about 4.26 weight percent of proteins, amino acids, or proteins and amino acids.

216. The nutritional supplement of claim 158, wherein the nutritional supplement contains a blend of the one or more proteins, amino acids, or proteins and amino acids that includes sodium caseinate in an amount ranging from about 1.3 to about 2.5 weight percent of the total blend, calcium sodium caseinate in an amount ranging from about 1.3 to about 2.5 weight percent of the total blend, soy protein isolate in an amount ranging from about 0.6 to about 2.5 weight percent of the total blend and one or more amino acids in an amount ranging from about 0.001 to about 2.0 weight percent of the total blend.

217. The nutritional supplement of claim 158 wherein the nutritional supplement has a viscosity that ranges from about 20 cp to about 45 cp.

218. The nutritional supplement of claim 217 wherein the nutritional supplement has a viscosity that ranges from about 30 cp to about 44 cp.

219. The nutritional supplement of claim 158, wherein one eight-fluid ounce serving of the nutritional supplement contains a number of calories ranging from about 100 to about 400 calories, and wherein about 70 of the calories are derived from fats, oils or a combination of fats and oils.

220. The nutritional supplement of claim 158, wherein a percent of the total caloric value obtained from fats, oils or fats and oils ranges from about 15% to about 45%.

221. The nutritional supplement of claim 220, wherein a percent of the total caloric value obtained from fats, oils or fats and oils ranges from about 28% to about 35%.

222. The nutritional supplement of claim 221, wherein a percent of the total caloric value obtained from fats, oils or fats and oils is about 32%.

223. The nutritional supplement of claim 158, wherein:
(a) about 10% or fewer of total calories are derived from saturated fat;
(b) from about 5% to about 10% of total calories are derived from n-6 polyunsaturated fatty acids;
(c) from about 15% to about 25% of total calories are derived from monounsaturated fat; and
(d) about 3% or fewer of total calories are derived from trans fatty acids.

224. The nutritional supplement of claim 158, wherein:
(a) about 3% or fewer of total calories are derived from saturated fat;
(b) about 7% of total calories are derived from n-6 polyunsaturated fatty acids;
(c) about 23% of total calories are derived from monounsaturated fat; and
(d) about 0.5% or fewer of total calories are derived from trans fatty acids.

225. The nutritional supplement of claim 158, wherein a percent of total caloric value of the nutritional supplement that is obtained from the one or more carbohydrates ranges from about 30% to about 65%.

226. The nutritional supplement of claim 225, wherein a percent of the total caloric value of the nutritional supplement that is obtained from the one or more carbohydrates ranges from about 45% to about 55%.

227. The nutritional supplement of claim 226, wherein a percent of the total caloric value of the nutritional supplement that is obtained from the one or more carbohydrates is about 51%.

228. The nutritional supplement of claim 158, wherein a percent of the total caloric value of the nutritional supplement that is obtained from the one or more additional proteins, amino acids, or proteins and amino acids, ranges from about 7% to about 30%.

229. The nutritional supplement of claim 228, wherein a percent of the total caloric value of the nutritional supplement that is obtained from the one or more additional proteins, amino acids, or proteins and amino acids, ranges from about 13% to about 20%.

230. The nutritional supplement of claim 229, wherein a percent of the total caloric value of the nutritional supplement that is obtained from the one or more additional proteins, amino acids, or proteins and amino acids is about 17%.

231. The nutritional supplement of claim 158, wherein total calories of the nutritional supplement that are derived from a combination of proteins, amino acids and carbohydrates is less than about 80%.

232. The nutritional supplement of claim 231, wherein total calories of the nutritional supplement that are derived from a combination of proteins, amino acids and carbohydrates is less than about 75%.

233. The nutritional supplement of claim 232, wherein total calories of the nutritional supplement that are derived from a combination of proteins, amino acids and carbohydrates is less than about 70%.

234. The nutritional supplement of claim 158, wherein a percent of the total caloric value of the nutritional supplement obtained from the one or more carbohydrates is from about 45% to about 75% greater than a percent of the total caloric value of the nutritional supplement obtained from the one or more fats, oils or combination of fats and oils.

235. The nutritional supplement of claim 158, wherein a caloric distribution of the nutritional supplement comprises the one or more fats, oils or fats and oils in a combined amount ranging from about 20% to about 40%, the one or more carbohydrates in a combined amount ranging from about 40% to about 60% and the one or more proteins, amino acids or proteins and amino acids in a combined amount ranging from about 10% to about 25%.

236. The nutritional supplement of claim 158, wherein the one or more antioxidants are present in a combined amount of about 0.1 weight percent.

237. The nutritional supplement of claim 158, wherein the one or more antioxidants are ascorbyl palmitate, tocopherol, citric acid, ascorbic acid, vitamin A, vitamin C, vitamin E, beta-carotene, selenium or magnesium.

238. The nutritional supplement of claim 158, wherein the one or more mouthfeel agents comprise a mixture of gum arabic and carrageenan.

239. The nutritional supplement of claim 158, wherein the one or more mouthfeel agents are present in a combined amount ranging from about 0.2 to about 2.0 weight percent of a total weight of the nutritional supplement.

240. The nutritional supplement of claim 239, wherein the one or more mouthfeel agents are present in a combined amount ranging from about 0.5 to about 1.0 weight percent of a total weight of the nutritional supplement.

241. The nutritional supplement of claim 158, wherein the one or more emulsifiers are present in a combined amount ranging from about 0.2 to about 2.0 weight percent of a total weight of the nutritional supplement.

242. The nutritional supplement of claim 241, wherein the one or more emulsifiers are present in a combined amount ranging from about 0.5 to about 1.0 weight percent of a total weight of the nutritional supplement.

243. The nutritional supplement of claim 158, wherein the one or more emulsifiers are egg yolk, egg lecithin, soy lecithin, mono-glycerides or di-glycerides.

244. The nutritional supplement of claim 243, wherein the one or more emulsifiers include soy lecithin.

245. The nutritional supplement of claim 158, wherein the nutritional supplement is flavored with Sucralose in an amount that is effective for providing a pleasant taste to, or enhancing the taste of, the nutritional supplement.

246. The nutritional supplement of claim 158, wherein the nutritional supplement is in form of a drink or shake.

247. The nutritional supplement of claim 158, wherein the nutritional supplement may be orally consumed by a human.

248. The nutritional supplement of claim 158, wherein the nutritional supplement may be administered to a human enterally.

249. The nutritional supplement of claim 248, wherein the nutritional supplement may be administered to a human parenterally.

250. The nutritional supplement of claim 158, wherein the nutritional supplement has a caloric content ranging from about 100 to about 400 calories per serving.

251. The nutritional supplement of claim 250, wherein the nutritional supplement has a caloric content ranging from about 200 to about 250 calories per serving.

252. The nutritional supplement of claim 158 wherein the one or more carbohydrates are in a form of a carbohydrate blend, and wherein the carbohydrate blend includes about 49% maltodextrin (10DE), about 23% crystalline fructose, about 20% maltitol, about 3% maltodextrin (digestive resistant dextrin—Fibersol 2), about 2% gum arabic and about 3% cellulose gel.

253. The nutritional supplement of claim 158 wherein the one or more fats, oils or fats and oils is in a form of a fat blend, and wherein the fat blend includes about 9% of saturated fat, about 69% of monounsaturated fat and about 22% of polyunsaturated fat.

254. The nutritional supplement of claim 158 wherein a percent of the total caloric value obtained from the one or more proteins, amino acids or proteins and amino acids is about 17%, a percent of the total caloric value obtained from the one or more carbohydrates is about 51% and a percent of the total caloric value obtained from the one or more fats, oils or fats and oils is about 32%.

255. The nutritional supplement of claim 158 wherein a percent of total formula calories derived from saturated fat is about 3%, a percent of total formula calories derived from polyunsaturated fat is about 5%, a percent of total formula calories derived from trans fatty acids is less than about 0.5% and a percent of total formula calories derived from monounsaturated fatty acids is about 23%.

256. The nutritional supplement of claim 158, wherein the nutritional supplement includes from about 400 IU beta-carotene and about 800 IU vitamin A as palmitate to about 1000 IU beta-carotene and about 2000 vitamin A IU as palmitate per day.

257. The nutritional supplement of claim 256, wherein the nutritional supplement includes from about 600 IU beta-carotene and about 1000 IU vitamin A as palmitate to about 750 IU beta-carotene and about 1400 IU vitamin A as palmitate per day.

258. The nutritional supplement of claim 257, wherein the nutritional supplement includes about 700 IU beta-carotene and about 1200 IU vitamin A as palmitate per day.

259. The nutritional supplement of claim 158, wherein the folic acid is present in an amount ranging from about 0.2 mg to about 1.0 mg per day.

260. The nutritional supplement of claim 259, wherein the folic acid is present in an amount ranging from about 0.4 mg to about 0.8 mg per day.

261. The nutritional supplement of claim 158, wherein the nutritional supplement includes at least about 100% U.S. RDI for folic acid.

262. The nutritional supplement of claim 158, wherein the nutritional supplement contains from about 0% to about 75% of minimum U.S. RDI for iron.

263. The nutritional supplement of claim 262, wherein the nutritional supplement contains from about 15% to about 35% of minimum U.S. RDI for iron.

264. The nutritional supplement of claim 158, wherein the nutritional supplement additionally contains chromium in a daily quantity ranging from about 60 mcg to about 500 mcg.

265. The nutritional supplement of claim 158, wherein the nutritional supplement additionally contains chromium in an amount of at least about 100% of the U.S. RDI for chromium.

266. The nutritional supplement of claim 158, wherein the nutritional supplement additionally contains chromium, and wherein one 8-fluid ounce serving of the nutritional supplement contains an amount of chromium ranging from about 0.05 mg to about 0.20 mg.

267. The nutritional supplement of claim 266, wherein the one 8-fluid ounce serving contains about 0.12 mg of chromium.

268. The nutritional supplement of claim 158, wherein the nutritional supplement additionally contains selenium in an amount that is effective for enhancing a metabolism of fat in a human or having activity as an antioxidant.

269. The nutritional supplement of claim 268, wherein the selenium is in the form of sodium selenite, and the nutritional supplement does not include sodium selenate.

270. The nutritional supplement of claim 269, wherein the sodium selenite is present in an amount ranging from about 10 mcg to about 200 mcg per day.

271. The nutritional supplement of claim 270, wherein the sodium selenite is present in an amount ranging from about 20 mcg to about 70 mcg per day.

272. The nutritional supplement of claim 158, wherein the L-arginine is present in an amount ranging from about 20 mg to about 3 g per day.

273. The nutritional supplement of claim 272, wherein the L-arginine is present in an amount ranging from about 500 mg to about 1.5 g per day.

274. The nutritional supplement of claim 273, wherein the L-arginine is present in an amount of about 1 g per day.

275. The nutritional supplement of claim 158, wherein the L-carnitine is present in an amount ranging from about 10 mg to about 150 mg per day.

276. The nutritional supplement of claim 275, wherein the L-carnitine is present in an amount ranging from about 30 mg to about 90 mg per day.

277. The nutritional supplement of claim 158, wherein one 8-fluid ounce serving of the nutritional supplement contains about 31 mg of L-carnitine.

278. The nutritional supplement of claim 158, wherein the nutritional supplement additionally contains m-inositol in an amount ranging from about 20 mg to about 400 mg per day.

279. The nutritional supplement of claim 278, wherein the m-inositol is present in an amount ranging from about 100 mg to about 200 mg per day.

280. The nutritional supplement of claim 158, wherein one 8-fluid ounce serving of the nutritional supplement contains about 186 mg of m-inositol.

281. The nutritional supplement of claim 158, wherein one 8-fluid ounce serving of the nutritional supplement contains about 1.20 weight percent of fiber of a total weight of the nutritional supplement.

282. The nutritional supplement of claim 158, wherein the nutritional supplement has a total solids content ranging from about 70 to about 95 percent by weight.

283. The nutritional supplement of claim 158, wherein the nutritional supplement can serve as a sole source of nutrition for a human.

284. The nutritional supplement of claim 158, wherein the nutritional supplement cannot serve as a sole source of nutrition for a human.

285. The nutritional supplement of claim 158, wherein the nutritional supplement does not contain any detectable level of gellan gum.

286. The nutritional supplement of claim 158, wherein the nutritional supplement does not contain any detectable level of taurine.

287. The nutritional supplement of claim 158, wherein the nutritional supplement does not contain any detectable level of soy polysaccharide.

288. The nutritional supplement of claim 158, wherein the nutritional supplement does not include insulin.

289. The nutritional supplement of claim 158, wherein one 8-fluid ounce serving of the nutritional supplement includes the following ingredients, and amounts thereof:

| Ingredients | Formula % by Weight |
|---|---|
| Water | 78.060 |
| Maltodextrin | 5.870 |
| High Oleic Sunflower Oil | 2.440 |
| Crystalline Fructose | 2.420 |
| Maltitol Syrup | 2.810 |
| Sodium Caseinate | 1.690 |
| Calcium Sodium Caseinate | 1.690 |
| Soy Protein Isolate | 0.880 |
| Fructooligosaccharides | 0.440 |
| Vitamin/Mineral Mix | 0.790 |
| Fibersol2 - Maltodextrin | 0.320 |
| High Oleic Canola Oil | 0.330 |
| Gum Arabic | 0.240 |
| Cellulose Gum/Gel | 0.400 |
| Soy Lecithin | 0.170 |
| Potassium Chloride | 0.100 |
| Docosahexaenoic acid (DHA) | 0.230 |
| Potassium Citrate | 0.080 |
| Sodium Citrate | 0.080 |
| Carrageenan | 0.020 |
| Sucralose | 0.030 |
| Potassium Phosphate | 0.100 |
| Natural and Artificial French Vanilla Flavor | 0.250 |
| Natural and Artificial Vanilla Flavor | 0.100 |
| Tricalcium Phosphate | 0.100 |
| Magnesium Phosphate | 0.240 |
| Citric Acid | 0.120 |
| Total | 100% |

290. The nutritional supplement of claim 289, wherein the one 8-fluid ounce serving contains 220 calories, and wherein about 70 of the calories are provided by the one or more fats, oils or fats and oils.

291. The nutritional supplement of claim 290, wherein the one 8-fluid ounce serving contains the following weight percents of the one or more proteins, amino acids or proteins and amino acids, the one or more carbohydrates and the one or more fats, oils or fats and oils:
(a) about 4% of the one or more proteins, amino acids or proteins and amino acids;
(b) about 12% of the one or more carbohydrate; and
(c) about 4% of the one or more fats, oils or fats and oils.

292. The nutritional supplement of claim 291, wherein the one or more carbohydrates are present in a carbohydrate blend that includes:
(a) about 49% maltodextrin (10DE);
(b) about 23% crystalline fructose;
(c) about 20% maltitol;
(d) about 3% maltodextrin (digestive resistant dextrin—Fibersol 2);
(e) about 2% gum arabic; and
(f) about 3% cellulose gel.

293. The nutritional supplement of claim 292, wherein the one or more fats, oils or fats and oils are present in a fat blend that includes:
(a) about 9% of saturated fat;
(b) about 69% of monounsaturated fat; and
(c) about 22% of polyunsaturated fat.

294. The nutritional supplement of claim 293, wherein a percent of total caloric value obtained from the one or more proteins, amino acids or proteins and amino acids in the nutritional supplement is about 17%, a percent of total caloric value obtained from the carbohydrate blend is about 51%, a percent of total caloric value obtained from the fat blend is about 32%, total calories derived from saturated fat is about 3%, total calories derived from polyunsaturated fat is about 5%, a percent of the total calories derived from trans fatty acids is less than about 0.5%, a percent of the total calories derived from monounsaturated fatty acids is about 23%, and a dextrose equivalence of starch employed is 10.

295. The nutritional supplement of claim 294, wherein the nutritional supplement has a viscosity of about 38.2 cp.

296. The nutritional supplement of claim 295, wherein the nutritional supplement has a total solids content of about 21.94% by weight.

297. The nutritional supplement of claim 296, wherein the nutritional supplement includes about 4 weight percent of the one or more proteins, amino acids or proteins and amino acids, about 12 weight percent of the one or more carbohydrates and about 4 weight percent of the one or more fats, oils or fats and oils.

298. A nutritional supplement formulation for glucose intolerant patients consisting essentially of:
(a) docosahexaenoic acid (DHA);
(b) L-arginine;
(c) lutein;
(d) one or more edible fats and/or oils in an amount sufficient to provide between about 15 to about 45% of the total caloric value of the formulation;
(e) carbohydrates in an amount sufficient to provide from about 30% to about 65% of the patient's daily energy requirements;
(f) proteins and/or amino acids in an amount sufficient to provide from about 7% to about 30% of the human's daily energy requirements;
(h) an artificial sweetener in an amount to impart a desired sweetness to the formulation; and
(i) a mineral and vitamin package containing minerals and vitamins in amounts sufficient to provide at least a portion of the recommended daily value (RDV) of each mineral and vitamin in the package.

299. The formulation of claim 298, wherein the at least one or more edible fats and/or oils are present as a fat blend which consists essentially of:
(i) saturated fat in an amount from about 5 to about 14 weight percent of the total fat blend;
(ii) monounsaturated fat in an amount from about 30 to about 80 weight percent of the total fat blend; and
(iii) polyunsaturated fat in an amount from about 10 to about 40 weight percent of the total fat blend.

300. The formulation of claim 298, wherein the carbohydrates are present as a carbohydrate blend which consists essentially of:
(i) maltodextrin (10DE) in an amount from about 40 to about 60 weight percent of the total carbohydrate blend;
(ii) crystalline fructose in an amount from about 15 to about 35 weight percent of the total carbohydrate blend;
(iii) maltitol in an amount from about 5 to about 30 weight percent of the total carbohydrate blend;
(iv) maltodextrin (Fibersol 2) in an amount from about 0.25 to about 6 weight percent of the total carbohydrate blend;
(v) gum arabic in an amount generally ranging from about 0.25 to about 6 weight percent of the total carbohydrate blend; and
(vi) cellulose gel in an amount from about 8 to about 35 weight percent of the total carbohydrate blend.

301. The formulation of claim 298, wherein the percent of the total caloric value thereof derived from protein is between about 23% to about 20%.

302. The formulation of claim 301, further comprising at least one selected from the group consisting of sodium caseinate in an amount from about 1.3 to about 2.5 weight percent of the total proteins, calcium sodium caseinate in an amount from about 1.3 to about 2.5 weight percent of the total proteins, soy protein isolate in an amount from about 0.6 to about 2.5 weight percent of the total proteins and added amino acids in an amount from about 0.001 to about 2.0 weight percent of the total proteins
